(19) United States
(12) Patent Application Publication
(10) Pub. No.: US 2011/0014196 A1
(43) Pub. Date: Jan. 20, 2011

(54) Drug Transfer into Living Cells

(75) Inventors: Andreas Brecht, Muttenz (CH); Florent Beaufils, Rosenau (FR)

Correspondence Address: HARRIET M. STRIMPEL, D. Phil., New England Biolabs, Inc., 240 COUNTY ROAD, IPSWICH, MA, 01938-2723, US

(73) Assignee: COVALYS BIOSCIENCES AG, Witterswil (CH)

(21) Appl. No.: 12/681,442

(22) PCT Filed: Oct. 2, 2008
(86) PCT No.: PCT/EP08/63205

§ 371 Date: Oct. 5, 2010

(30) Foreign Application Priority Data
Oct. 3, 2007 (EP) 07117800.8
Jul. 10, 2008 (EP) 08104709.4

(51) Int. Cl. A61K 39/395; C12N 9/10; A61P 35/00; C07D 473/18; C07H 21/02; C07H 19/167; A61K 31/713; A61K 31/522; A61K 31/7076; A61K 31/538; A61K 38/45

(52) U.S. Cl. 424/134.1; 435/193; 544/276; 536/24.5; 536/26.23; 544/99; 514/44 A; 514/263.21; 514/47; 514/229.5; 424/94.5

(57) ABSTRACT

The invention relates to compounds comprising a plurality of enzyme substrates suitably linked and further carrying one or more cargo entities. In particular such compounds have the structure (substrate)ₙ-linker-(cargo)ₘ wherein “substrate” is a substrate specific for an enzyme-type protein; n is 2 or more; “linker” is a linking unit consisting of 1 to 300 atoms; “cargo” is a drug, a label detectable by a fluorescence detector, magnetic resonance imaging (MRI), positron emission tomography (PET) or scintigraphy, or a functional group which can be transformed into a drug or a detectable label; and m is 1 or more. The invention further relates to a corresponding molecular shuttles having the structure (fusion protein)ₙ-linker-(cargo)ₘ wherein “fusion protein” is a proteinaceous binding entity fused to an enzyme-type protein for which specific substrates exist. The proteinaceous binding entity is designed to bind to a target structure in vitro or in vivo, for example a cellular receptor.

### FIELD OF THE INVENTION

The present invention relates to compounds suitable for transferring a drug or a detectable label from substrates first to fusion proteins directed to a target and then to the target, and corresponding methods.

### BACKGROUND OF THE INVENTION

There is a constant need for improved techniques to direct a drug or imaging agent to the desired site of action. Desirable features of molecular shuttles, i.e. compounds for directing drugs to the desired site of action, are low immunogenicity, high target specificity and high avidity. Standard molecular shuttles for such applications are antibodies, in particular humanized antibodies carrying the corresponding drug or imaging agent.

Methods are known for specific labelling a protein of interest under in vitro or in vivo conditions. One particular method is disclosed in WO 02/083937 describing a method for detecting and/or manipulating a protein of interest wherein the protein is fused to O⁶-alkylguanine-DNA alkyltransferase (AGT) and the AGT fusion protein contacted with a specific AGT substrate based on O⁶-benzylguanine carrying a label, whereby the label is transferred to the fusion protein. The AGT fusion protein is then detected and optionally further manipulated using the label. Several mutants of wild type AGT were shown to be better suitable than wild type AGT (WO 2004/031404; WO 2005/085431) in such a labelling method, and a wide range of substituted benzylguanines and related heteroarylmethylguanine compounds were described for use in transferring a label to the fusion proteins comprising AGT and AGT mutants (WO 2004/031405; WO 2005/085470).

A more recent variant of such a method is described in PCT/EP2007/057597, wherein substrates based on O²-benzyl-cytosines carrying a label specifically transfer this label to proteins called alkylcytosine transferases (ACTs) derived from O⁶-alkylguanine-DNA alkyltransferase, and to fusion proteins comprising such ACT.

Another suitable system of transferring a label to a fusion protein is described in WO2004/104588. A fusion protein comprising protein of interest and an acyl carrier protein (ACP) or a fragment thereof is contacted with a labeled coenzyme A (CoA) type substrate and a holo-acyl carrier protein synthase (ACPS) or a homologue thereof so that the ACPS transfers the label to the fusion protein.

In WO 2004/072232, mutant deshalogenase and chloroalkane derivatives suitable as substrates for transferring a label to such modified deshalogenase, and also mutant serine beta-lactamase and corresponding substrates transferring a label to such mutant beta-lactamase are described.

### SUMMARY OF THE INVENTION

The invention relates to compounds comprising a plurality of enzyme substrates suitably linked and further carrying one or more cargo entities.

In particular such compounds have the structure

(substrate)ₙ-linker-(cargo)ₘ

wherein
“substrate” is a substrate specific for an enzyme-type protein or several different substrates specific for same or different enzyme-type proteins;
n is 2 or more, for example 2, 3, 4 or 5, in particular 2 or 3;
“linker” is a linking unit consisting of 1 to 300 carbon atoms, wherein up to a third of the carbon atoms may be replaced by oxygen atoms and/or nitrogen atoms and/or one or more carbon atoms may be replaced by sulfur atoms, may be linear or branched and/or comprise double bonds, triple bonds, carbocycles or heterocycles, and may carry further substituents, in particular an oxo group on a carbon atom adjacent to a nitrogen atom or an oxygen atom;
“cargo” is a drug, a label detectable by a fluorescence detector, magnetic resonance imaging (MRI), positron emission tomography (PET) or scintigraphy, or a functional group which can be transformed into a drug or a detectable label, whereby several “cargo” entities may be the same or different drug, detectable label or functional group; and
m is 1 or more, for example 1, 2, 3 or 4, in particular 1 or 2.

The invention further relates to a molecular shuttle comprising fusion proteins carrying one or more cargo entities.

In particular such molecular shuttles have the structure

(fusion protein)ₙ-linker-(cargo)ₘ

wherein “fusion protein” is a proteinaceous binding entity fused to an enzyme-type protein for which specific substrates exist; and n, linker, cargo and m are defined as hereinbefore. The proteinaceous binding entity is designed to bind to a target structure in vitro or in vivo, for example a cellular receptor. A particular binding entity is a recombinant antibody fragment.

The invention further relates to novel fusion proteins comprising a proteinaceous binding entity fused to an enzyme-type protein which reacts covalently with a specific substrate or part of a specific substrate through a particular amino acid residue, this reaction being promoted either by the enzyme-type protein itself or by the auxiliary activity of a synthase enzyme promoting the formation of the bound state between part of the substrate and a specific amino acid of the enzyme-type protein.

Moreover the invention relates to a method of reacting a compound comprising a plurality of enzyme substrates suitably linked and further carrying one or more cargo entities with fusion proteins comprising a proteinaceous binding entity fused to the enzyme-type protein or enzyme-type proteins for which the substrates are specific.

Furthermore the invention relates to pharmaceutical compositions comprising the molecular shuttles as defined hereinbefore, wherein at least one of the cargo entities is a drug, and to a method of treatment comprising administering a molecular shuttle or a pharmaceutical composition comprising a molecular shuttle as defined hereinbefore, wherein at least one of the cargo entities is a drug.

### DETAILED DESCRIPTION OF THE INVENTION

The invention relates to compounds comprising a plurality of enzyme substrates suitably linked and further carrying one or more cargo entities. In particular such compounds have the structure (substrate)ₙ-linker-(cargo)ₘ.

“Substrate” is a substrate specific for an enzyme-type protein or several different substrates specific for same or different enzyme-type proteins. Several examples of enzymes and specific substrates for such enzymes are known and considered in the present invention. Examples are:

(a) Alkylguanine-DNA-alkyltransferase (AGT) and substrates, which are O⁶-alkylguanine derivatives, for example O⁶-benzylguanine derivatives or O⁶-heteroarylmethylguanine derivatives. These substrates are then modified in order to couple them to a suitable linker. AGT substrates and their use in transferring a label to the enzyme AGT are described in WO 2005/085470 and earlier publications cited therein, and such substrates are also considered here. Other suitable selective substrates are 2-amino-4-benzyloxypyrimidines described in WO 2006/114409. Most preferred as substrates are O⁶-benzylguanines further substituted in position 4 of the benzyl function, which are selective for AGT, for example human AGT or for particular modified AGT type enzymes obtained by mutation form wild type AGT according to the methods described in WO 2005/085431 and earlier publications cited therein.

(b) Alkylcytosine transferase (ACT) and substrates, which are O²-benzyl-cytosine derivatives and related O²-heteroarylmethyl-cytosine derivatives. These substrates are then modified in order to couple them to a suitable linker. ACT substrates and their use in transferring a label to the enzyme ACT are described in PCT/EP2007/057597, and such substrates are also considered here. Most preferred as substrates are O²-benzyl-cytosines further substituted in position 4 of the benzyl function selective for ACT, which is a modified AGT type enzyme obtained by directed mutation from AGT.

(c) Acyl carrier protein (ACP) and substrates, which are coenzyme A derivatives and related compounds. The phosphopantetheine subunit of a coenzyme A is suitably modified in order to couple it to a linker. Coenzyme A derivatives and their use in transferring a label to the enzyme-type protein ACP or a fragment thereof in the presence of a holo-acyl carrier protein synthase (ACPS) or a homologue thereof are described in WO 2004/104588, and such substrates are also considered here.

(d) Mutant deshalogenase and substrates, which are chloroalkane derivatives. Chloroalkane derivatives and their use in transferring a label to an omega-carboxyl group of an aspartic acid residue or a glutamic acid residue of a suitable modified deshalogenase are described in WO 2004/072232, and such substrates are also considered here.

(e) Mutant serine beta-lactamase and corresponding substrates, which form a stable bond with a serine beta-lactamase. Such selective substrates are likewise described in WO 2004/072232 and are also considered here.

n is 2 or more, for example 2, 3, 4 or 5, in particular 2 or 3.

The two or more substrates connected to the linker may be identical or different. Different substrates connected to the linker may have selectivity for the same enzyme-type protein or may be substrates selective for different enzyme-type proteins. Preferred are, for example, two or three identical O⁶-benzylguanine derivative substrates connected to the linker. Alternatively, one substrate may be an O⁶-benzylguanine derivative selective for AGT, and another substrate bound to the same linker an O²-benzyl-cytosine derivative selective for ACT. Any combination of substrates and enzyme selectivity described above may be used.

“linker” is a linking unit consisting of 1 to 300 carbon atoms, wherein up to a third of the carbon atoms may be replaced by oxygen atoms and/or nitrogen atoms and/or one or more carbon atoms may be replaced by sulfur atoms, may be linear or branched and/or comprise double bonds, triple bonds, carbocycles or heterocycles, and may carry further substituents, in particular an oxo group on a carbon atom adjacent to a nitrogen atom or an oxygen atom.

The linker is preferably a flexible linker connecting cargo to the 2 or more substrates. Linker units are chosen in the context of the envisioned application, i.e. in the transfer of the cargo to a fusion protein comprising the enzyme-type proteins for which the substrates are selective. They also increase the solubility of the substrates and cargo in the appropriate solvent. The linkers used are chemically stable under the conditions of the actual application. The linkers do not interfere with the reaction of the substrates with the enzymes nor with the function of the cargo.

In particular, a linker is a straight or branched chain alkylene group with 1 to 300 carbon atoms, wherein optionally

(a) one or more carbon atoms are replaced by oxygen, in particular wherein every third carbon atom is replaced by oxygen, e.g. a poylethyleneoxy group with 1 to 100 ethyleneoxy units;
(b) one or more carbon atoms are replaced by nitrogen carrying a hydrogen atom or further substituent, representing an amine function, or, in the case that the adjacent carbon atom is substituted by oxo, an amide function —NH—CO—, or, if two adjacent carbon atoms are replaced by nitrogen atoms, a hydrazine function —NH—NH— or a carbonylhydrazine function —NH—NH—CO—;
(c) one or more carbon atoms are replaced by oxygen, and the adjacent carbon atoms are substituted by oxo, representing an ester function —O—CO—;
(d) the bond between two adjacent carbon atoms is a double or a triple bond, representing a function —CH═CH— or —C≡C—, or the bond between a carbon and a nitrogen atom is a double bond representing an imine —C(R)═N— or a hydrazone —C(R)═N—NH—, or the bond between two adjacent nitrogen atoms is a double bond representing a diazo group —N═N—;
(e) one or more carbon atoms are replaced by a phenylene, a saturated or unsaturated cycloalkylene, a saturated or unsaturated bicycloalkylene, a bridging heteroaromatic or a bridging saturated or unsaturated heterocyclyl group;
(f) one or more carbon atoms are replaced by a sulfur atom, representing a thioether or, if two adjacent carbon atoms are replaced by sulfur atoms, a disulfide linkage —S—S—; or a combination of two or more, especially two or three, alkylene and/or modified alkylene groups as defined under (a) to (f) hereinbefore, optionally containing substituents.

Substituents considered are e.g. lower alkyl, e.g. methyl, hydroxy, lower alkoxy, e.g. methoxy, lower acyloxy, e.g. acetoxy, amino, lower acylamino, e.g. acetylamino or trifluoroacetylamino, halogenyl, e.g. chloro, or oxo.

Further substituents considered are e.g. those obtained when an α-amino acid, in particular a naturally occurring α-amino acid, is incorporated in the linker, wherein carbon atoms are replaced by amide functions —NH—CO— as defined under (b). In such a linker, part of the carbon chain of the alkylene group is replaced by a group —(NH—CHR—CO)ₓ— wherein x is between 1 and 100 and R represents a varying residue of an α-amino acid.

A phenylene group replacing carbon atoms as defined under (e) hereinbefore is e.g. 1,2-, 1,3-, or preferably 1,4-phenylene. A saturated or unsaturated cycloalkylene group replacing carbon atoms as defined under (e) hereinbefore is derived from cycloalkyl with 3 to 7 carbon atoms, preferably from cyclopentyl or cyclohexyl, and is e.g. 1,2- or 1,3-cyclopentylene, 1,2-, 1,3-, or preferably 1,4-cyclohexylene, or also 1,4-cyclohexylene being unsaturated, e.g. in 1- or in 2-position. A saturated or unsaturated bicycloalkylene group replacing carbon atoms as defined under (e) hereinbefore is derived from bicycloalkyl with 7 or 8 carbon atoms, and is e.g. bicyclo[2.2.1]heptylene or bicyclo[2.2.2]octylene, preferably 1,4-bicyclo[2.2.1]heptylene optionally unsaturated in 2-position or doubly unsaturated in 2- and 5-position, and 1,4-bicyclo[2.2.2]octylene optionally unsaturated in 2-position or doubly unsaturated in 2- and 5-position. A bridging heteroaromatic group replacing carbon atoms as defined under (e) hereinbefore is e.g. triazolidene, preferably 1,4-triazolidene, or isoxazolidene, preferably 3,5-isoxazolidene. A bridging saturated or unsaturated heterocyclyl group replacing carbon atoms as defined under (e) hereinbefore is e.g. derived from an unsaturated heterocyclyl group, e.g. 3,5-isoxazolidinene, or a fully saturated heterocyclyl group with 3 to 12 atoms, 1 to 3 of which are heteroatoms selected from nitrogen, oxygen and sulfur, e.g. pyrrolidinediyl, piperidinediyl, tetrahydrofuranediyl, dioxanediyl, morpholinediyl or tetrahydrothiophenediyl, preferably 2,5-dioxopyrrolidine-1,3-diyl(succinimido), 2,5-tetrahydrofuranediyl or 2,5-dioxanediyl. A particular heterocyclyl group considered is a saccharide moiety, e.g. an α- or β-furanosyl or α- or β-pyranosyl moiety, or a succinimido group.

A linker is preferably a straight chain or a doubly or triply branched chain alkylene group with 6 to 25 carbon atoms optionally comprising one or more, for example 1 to 6 amide functions —NH—CO—, or a straight chain or a doubly or triply branched chain polyethylene glycol group with 3 to 100 ethyleneoxy units, optionally comprising one or more, for example 1 to 6 amide functions —NH—CO—, a urea function —NH—CO—NH—, and optionally a thioether function and a succinimido group, i.e. a nitrogen containing five-membered heterocycle bound to the alkylene chain through the nitrogen atom and a carbon atom, and further substituted by two oxo groups at the two carbon atoms next to nitrogen. The thioether function is preferably connected to the succinimido group. Further preferred is a straight chain or branched linker comprising one or more polyethylene glycol groups of 3 to 20, preferably 3 to 12 ethylene glycol units and alkylene groups wherein carbon atoms are replaced by amide bonds, and further carrying substituted amino and hydroxy functions and/or thioether and succinimido groups. Other preferred branched linkers have dendritic (tree-like) structures wherein amine, carboxamide, ether and/or thioether functions replace carbon atoms of an alkylene group.

A particularly preferred linker is a doubly or triply branched chain alkylene group with 6 to 25 carbon atoms comprising one or more, for example 1 to 6 amide functions —NH—CO— and optionally a urea function —NH—CO—NH— and/or thioether and succinimido groups, or a doubly or triply branched chain polyethylene glycol group with 3 to 60, preferably 3 to 36 ethyleneoxy units comprising one or more, for example 1 to 10, such as 1 to 6 amide functions —NH—CO— and optionally a urea function —NH—CO—NH—, and/or thioether and succinimido groups.

Other preferred linkers are those comprising a disulfanyl function or a hydrazone function, for example a carbonylhydrazone function.

A preferred example of such a linker is derived from tris(hydroxymethyl)methylamine and has, for example, the structure [—(NHCOCH₂CH₂)ₚ(OCH₂CH₂)_{q}NHCOCH₂CH₂OCH₂]₃C—NHCO—, wherein p is 0 or 1 and q is 0 or between 1 and 20, for example between 3 and 15, such as 4 or 12, and wherein — indicates a bond to cargo.

Another preferred example of such a linker derived from tris(hydroxymethyl)methylamine has, for example, the structure [—(NHCOCH₂CH₂)ₚ(OCH₂CH₂)ₚOCOCH₂CH₂OCH₂]₃C—NHCO—, wherein p is 0 or 1 and q is 0 or between 1 and 20, for example between 3 and 15, such as 4 or 12.

Another preferred example of such a linker is derived from amino-substituted succinic acid diamide or glutaric acid diamide and has, for example the structure —HNCOCH₂(CH₂)ₚCH(NH—)CONH—, wherein p is 0 or 1, in particular —HNCONH(CH₂CH₂O)ᵣCH₂CH₂HNCOCH₂(CH₂)ₚCH(NH—)CONHCH₂CH₂(OCH₂CH₂)ᵣO—, wherein p is 0 or 1 and r is between 1 and 20, such as between 1 and 6, e.g. 3, and wherein — indicates a bond to cargo.

In particular the linker may contain a structure improving the endosomal release of cargo, taken up by a cell through internalization of the shuttle according to the invention. Preferred are intracellularly labile linkers, such as linkers comprising a disulfanyl function, a hydrazone or carbonylhydrazone function, carboxylic ester functions (which may be cleaved by intracellular esterases) or synthetic peptide functions (prone to degradation by intracellular peptidases and proteases). Such intracellular cleavage will promote release of the cargo from the endosomes or lysosomes, which is particularly preferred if the cargo is a drug.

Preferred examples of linkers are those mentioned above derived from tris(hydroxylmethyl)methylamine, amino-substituted succinic acid diamide or amino-substituted glutaric acid diamide further comprising an urea function —NHCONH—, a disulfanyl group —S—S—, or a hydrazone function —CR═N—NH— or —CR═N—NH—CO—, wherein R is H or, preferably, methyl. Particular examples of a sulfanyl group containing linkers are those comprising a group —CH₂CH₂—S—S—CH₂CH₂—, optionally connected via a carbonyl, amido or 2-thiosuccinimido function. Particular examples of a hydrazone group containing linkers are those comprising a group —C₆H₄—C(CH₃)═N—NH—CO—C₆H₁₀—CH₂—, optionally connected via a carbonyl, amido or 2-thiosuccinimido function.

Lower alkyl is alkyl with 1 to 7, preferably from 1 to 4 C atoms, and is linear or branched; preferably, lower alkyl is butyl, such as n-butyl, sec-butyl, isobutyl, tert-butyl, propyl, such as n-propyl or isopropyl, ethyl or methyl. Most preferably, lower alkyl is methyl.

In lower alkoxy, the lower alkyl group is as defined hereinbefore. Lower alkoxy denotes preferably n-butoxy, tert-butoxy, iso-propoxy, ethoxy, or methoxy, in particular methoxy.

In lower acyloxy or acylamino, lower acyl has the meaning of formyl or lower alkylcarbonyl wherein lower alkyl is defined as hereinbefore. Lower acyloxy denotes preferably n-butyroxy, n-propionoxy, iso-propionoxy, acetoxy, or formyloxy, in particular acetoxy. Lower acylamino is preferably acetylamino.

Halogen is fluoro, chloro, bromo or iodo, in particular chloro.

“cargo” is a drug, a label detectable by a fluorescence detector, magnetic resonance imaging (MRI), positron emission tomography (PET) or scintigraphy, or a functional group which can be transformed into a drug or a detectable label, whereby several “cargo” entities may be the same or different drug, detectable label or functional group.

Drugs considered as cargo are drugs which are more effective when transported to a particular site within the body. Examples are drugs which should interact with a particular cellular receptor or other entity, for example cytotoxic drugs which should be transported to the site of cancer cells. In particular, such drugs considered in the present application are chlorambucil, podophyllotoxin, methotrexate, topotecan hydrochloride, and camptothecin. Modified derivatives of vinca alkaloids (such as vincristine, vinblastine, vinorelbine and vindesine) and taxanes (such as paclitaxel, docetaxel, taxotere) are also considered.

Further examples of drugs are radioactive materials with short half life and limited penetration depth of the radiation emitted upon decay of the radioactive isotope, typically short lived emitters of alpha-radiation, for example derivatives of ⁹⁹Tc, ¹¹¹In, ²¹¹At, and ²¹²Bi from ²¹²Pb (see for example Fritzberg, A. R. in Journal of Nuclear Medicine 39:20 N, 1998).

Other examples of drugs considered are oligonucleotides, e.g. DNA or RNA strands with the ability to have a significant effect on the situation of the targeted cell, but also nucleic acid derivatives and analogues, e.g. compounds in which the sugar phosphate backbone is replaced by other units, such as e.g. amino acids (such compounds are denoted PNA and are described in WO 92/20702), more preferably RNAi, and most preferably precursors of siRNA or siRNA itself, preferably with the potential to downregulate a particular protein of interest, or to stop a certain metabolic pathway, e.g. to enhance the effect of a co-administered cytotoxic drug.

Labels detectable by a fluorescence detector, magnetic resonance imaging (MRI), positron emission tomography (PET) or scintigraphy as cargo are, for example, fluorophores, more preferably NIR fluorophores excitable between 650 nm and 950 nm, which are detectable in vitro and in vivo by fluorescence detection systems. Further detectable labels are iron oxide particles and other groups with high contrast for MRI imaging applications. Further detectable labels considered are radiopharmaceutical labels used for imaging, for example on the basis of technetium, Tc-99m.

A functional group which can be transformed into a drug or a detectable label considered as cargo is a protected reactive group, preferably one that can be easily deprotected before use and shows high reactivity towards reaction partners introducing an unstable drug, such as radioactive materials with short half life suitably complexed, or introducing a short-lived detectable radiopharmaceutical label for imaging purposes. Examples of such functional groups are protected amino functions, e.g. trifluoracetamides, easily deprotected and able to react with activated carboxylic acids. Another example is a lipoic acid derivative, which may be easily loaded with ⁷²As³⁺, which reaction is based on the reduction of a disulfide bond in the lipoic acid unit to a dithiol as described in U.S. Pat. No. 5,914,096. Once the dithiol is formed, addition of As³⁺ will result in formation of a covalently bound arsenic through two sulfur-arsenic bonds.

A further functional group which can be transformed into a drug or a detectable label considered as cargo is a group being able to complex radioactive metal isotopes, for example diethylenetriaminepentaacetic acid (DTPA), which is a widely used as organic ligand in magnetic resonance imaging (MRI) and positron emission tomography (PET).

Several “cargo” entities may be the same or different drug, detectable label or functional group. For example one cargo entity may be a cytotoxic drug and another cargo entity a siRNA increasing sensitivity towards the cytotoxic drug, or one cargo may be a drug and another cargo a detectable label, e.g. a fluorescent label.

Preferred cargo entities are drugs and detectable labels as defined hereinbefore.

m is 1 or more, for example 1, 2, 3 or 4, in particular 1 or 2.

Particular compounds of the structure (substrate)ₙlinker-(cargo)ₘ are, for example,

(a) a compound of the structure (substrate)₂-linker-drug;
(b) a compound of the structure (substrate)₃-linker-drug;
(c) a compound of the structure (substrate)₂-linker-(drug)(detectable label);
(d) a compound of the structure (substrate)₃-linker-(drug)(detectable label);
(e) a compound of the structure (substrate-1)(substrate-2)-linker-drug; and
(f) a compound of the structure (substrate)₂-linker-(drug-1)(drug-2).

Preferred are compounds of the structure (substrate)ₙ-linker-(cargo)ₘ as explained hereinbefore, wherein linker has one of the preferred meanings. Most preferred are compounds of the structure

[substrate-(NHCOCH₂CH₂)ₚ(OCH₂CH₂)_{q}NHCOCH₂CH₂OCH₂]₃C—NHCO-cargo,

wherein p is 0 or 1 and q is between 1 and 20, in particular such compounds, wherein substrate is O⁶-benzylguanine connected to the linker through a CH₂ group in para position of the benzyl function. Preferred values for q are 4 and 12, in particular 12. Cargo is as defined hereinbefore, preferably a drug or a detectable label, such as a fluorophore. Most preferred are the compounds of the Examples, such as a compound wherein p is 1 and q is 12.

The invention further relates to a molecular shuttle comprising fusion proteins carrying one or more cargo entities. In particular such molecular shuttles have the structure (fusion protein)ₙ-linker —(cargo)ₘ wherein “fusion protein” is a proteinaceous binding entity fused to an enzyme-type protein for which specific substrates exist; and substrate, n, linker, cargo and m are defined as hereinbefore.

The proteinaceous binding entity is designed to bind to a target structure in vitro or in vivo, for example a cellular receptor. Such a target structure may be inside or, preferably, on the surface of a target cell, and typically inside living multicellular organisms, preferably mammals, most preferably humans. Examples of proteinaceous binding entities are proteins, peptides, or glycoproteins.

A particular binding entity is an antibody or antibody fragment. Such antibodies and antibody fragments with selectivity for a particular target structure are well known in the art. Preferred are recombinant antibody fragments, and preferably humanized antibody fragments when an application in humans is intended. Antibody fragments may, for example, be Fab, Fab′ or preferably scFv fragments.

Alternatives to antibodies or antibody fragments known in the art are also considered, for example natural or fully synthetic binder proteins directed to particular receptors within cells or on cell membranes. Particular examples are listed e.g. by Fiedler M. et al. in TRENDS in Biotechnology, 23:514, 2005, and include, but are not limited to, three-helix bundles from Z-domain of Protein A from S. aureus; binders based on human transferrin; monomeric or trimeric human C-type lectin domains; and ankyrin repeat proteins.

The invention further relates to novel fusion proteins comprising a proteinaceous binding entity as described above fused to an enzyme-type protein for which specific substrates exist, in particular enzyme-type proteins described above such as AGT, ACT, ACP, deshalogenase, or serine beta-lactamase.

Moreover the invention relates to a method of reacting a compound comprising a plurality of enzyme substrates suitably linked and further carrying one or more cargo entities with fusion proteins comprising a proteinaceous binding entity fused to the enzyme-type protein or enzyme-type proteins for which the substrates are specific.

In such a method, a compound having the structure (substrate)ₙ-linker-(cargo)ₘ as described above is mixed with the fusion protein comprising the enzyme-type protein for which the substrate is specific, preferably in a four- to fivefold excess, preferably at a concentration of 10 μM or higher, in a suitable solvent, for example phosphate buffered saline at pH 7.4, optionally containing further components such as dithiothreitol, for extended period of time, for example 4 to 48 hours, and purifying the obtained reaction mixture by standard methods, for example gel permeation chromatography.

The molecular shuttles according to the invention show the desired property: If a microplate is modified by absorbing the protein targeted by the antibody subunit of the fusion proteins and a range of densities established according to standard ELISA protocols, then a molecular shuttle comprising three fusion proteins and a fluorescent label is incubated and the affinity compared to a fluorescently labelled single fusion protein, an affinity is found for the molecular shuttle of the invention, which is at least ten times higher than that of the single fusion protein.

Furthermore, cells expressing the target protein on their surface are incubated either with a single fusion protein modified with a fluorophore or with a molecular shuttle comprising three fusion proteins and a fluorescent label of the invention. Estimated from the fluorescence micrographs, the amount of fluorophore that is internalised and cannot be washed away is at least two times higher for the molecular shuttle of the invention than for the single fusion protein.

Furthermore the invention relates to pharmaceutical compositions comprising the molecular shuttles as defined hereinbefore, wherein at least one of the cargo entities is a drug, and to a method of cancer treatment comprising administering a molecular shuttle or a pharmaceutical composition comprising a molecular shuttle as defined hereinbefore, wherein at least one of the cargo entities is a drug useful in the treatment of cancer.

The prime application for pharmaceutical compositions of the invention is cancer where molecular shuttles will be administered to inhibit the growth of or to kill selectively cancer cells exhibiting a particular surface structure and showing abnormal growth. Further applications are in the prevention of the growth of harmful structures including one or several particular cell types without neoplastic characteristics, like in atherosclerotic processes, leading to stenosis of blood vessels.

### EXAMPLES

### Abbreviations

BC—NH2=2-(4-aminomethylbenzyloxy)-4-aminopyrimidine (aminomethylbenzylcytosine)
BG-PEG4-NH2=6-{4-[(2-{2-[2-(2-aminoethoxy)ethoxyethoxy]ethoxy}methyl]benzyloxy}-9H-purin-2-amine (pegylated O⁶-benzylguanine)
CDI=N,N′-carbonyl diimidazole
CoA-SH=coenzyme A
DCC=dicyclohexylcarbodiimide
DCU=dicyclohexylurea
DIPEA=diisopropylethylamine
DMF=dimethylformamide
DMSO=dimethyl sulfoxide
DTT=dithiothreitol
EDC=1-(3-(dimethylamino)propyl)-3-ethylcarbodiimide
eq=equivalent
ESI-MS=electrospray ionization mass spectrometry
Et₃N=triethylamine
EtOAc=ethyl acetate
EtOH=ethanol
FAB-MS=fast atom bombardment mass spectrometry
HOBT=1-hydroxybenzotriazole
HPLC=high pressure liquid chromatography
Lys=lysine
MeN H₂=methylamine
MeOH=methanol
NHS=N-hydroxy succinimide
NMP=N-methylpyrrolidine
PBS=phosphate buffered saline
PEG=polyethylene glycol
PEG12=—(CH₂CH₂O)₁₂—
PMe₃=trimethylphosphine
PYBOP=(benzotriazol-1-yloxy)-tripyrrolidino-phosphonium hexafluorophosphate
rt=room temperature
siRNA=short interfering ribonucleic acid
TFA=trifluoroacetic acid
Tris=tris(hydroxymethyl)methylamine

### Example 1

### Tris{[2-(tert-butoxycarbonyl)ethoxy]methyl}methylamine (1)

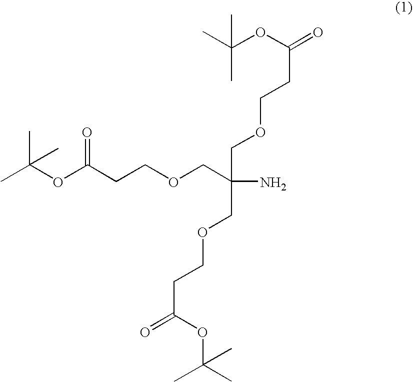

Tris(hydroxymethyl)methylamine (Tris, 2.42 g, 20.0 mmol) in 4.0 mL of a newly opened bottle of DMSO is cooled to 15.0° C. Then, OA mL of 5.0 M NaOH is injected while stirring, followed by tert-butyl acrylate (10.0 mL, 68 mmol), which is injected dropwise. A solvent mixture of 5-10% water in DMSO is optimal for this reaction. The reaction mixture is allowed to reach room temperature and left stirring for 24 h. Then the crude mixture is poured onto water and extracted with ethyl acetate, the organic phase is dried over MgSO₄, and evaporated under reduced pressure to afford (1). The compound is directly used for next step without further purification. FAB-MS: m/z 506 [M+H]⁺.

### Example 2

### N-Tris{[2-(tert-butoxycarbonyl)ethoxy]methyl}methyl trifluoroacetamide (2)

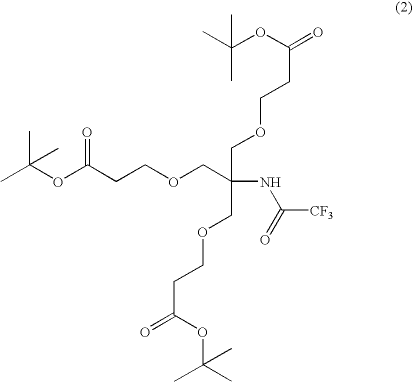

To a solution of tris{[2-(tert-butoxycarbonyl)ethoxy]methyl}methylamine (1) (10 mmol, 5.05 g) in methanol (30 mL) triethylamine (1 eq, 10 mmol, 1.39 mL) is added at rt. Then, ethyl trifluoroacetate (1.3 eq, 13 mmol, 1.55 mL) is slowly added over 20 min at rt. The reaction mixture is stirred overnight at rt. The solvent is evaporated, the residue is diluted with ethyl acetate (100 mL) and washed with a saturated solution of NaCl. The organic layer is dried over MgSO₄ and concentrated under reduce pressure. Flash chromatography (cyclohexane/ethyl acetate, 2/1→1/1) gives the desired compound (2). ESI-MS: m/z 602.31 [M+H]⁺.

### Example 3

### N-Tris{[2-carboxyethoxy]methyl}methyl trifluoroacetamide (3)

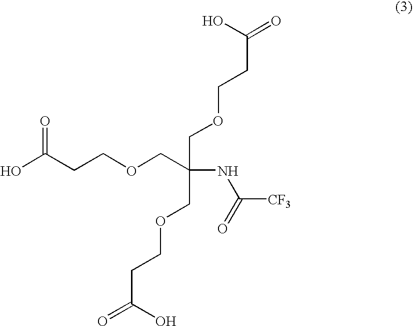

N-Tris{[2-(tert-butoxycarbonyl)ethoxy]methyl}methyl trifluoroacetamide (2) (4.81 g, 8 mmol) is stirred in 80 mL of 96% formic acid for 18 h. The formic acid is removed at reduced pressure at 50° C. to produce a colorless oil in quantitative yield.

ESI-MS: m/z 434.12 [M+H]⁺.

### Example 4

### N-Tris[2-(BG-PEG4-NH-carbonyl)ethyloxymethyl]methyl trifluoroacetamide (4)

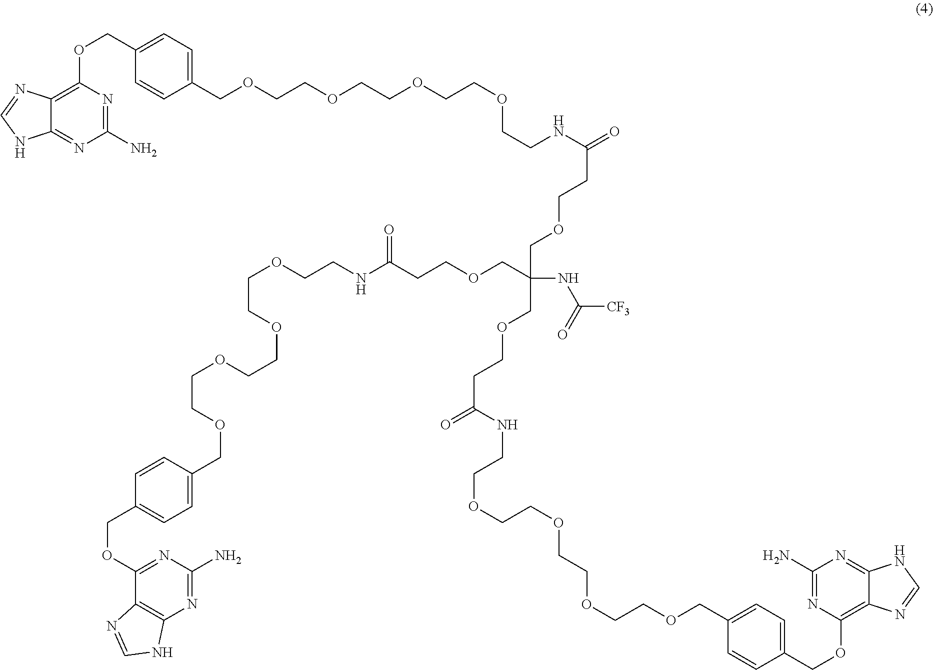

To a solution of (3) (433 mg, 1 mmol, 1 eq) and BG-PEG4-NH2 (1.34 g, 3 mmol, 3 eq) in DMF (10 mL) are successively added DIPEA (495 μL, 3 mmol, 3 eq), HOBT (1 M in NMP, 3 mL, 3 mmol, 3 eq) and DCC (620 mg, 3 mmol, 3 eq) at rt. The resulting mixture is stirred overnight. The solvent is removed under reduced pressure and the mixture is diluted with 250 mL of ethyl acetate. The organic layer is washed with water, dried over MgSO₄ and evaporated under reduced pressure. Flash chromatography (CH₂Cl₂/methanol, 10/1→5/1) gives the desired compound (4). ESI-MS: m/z 1718.77 [M+H]⁺.

### Example 5

### Tris[2-(BG-PEG4-NH-carbonyl)ethyloxymethyl]methylamine (5)

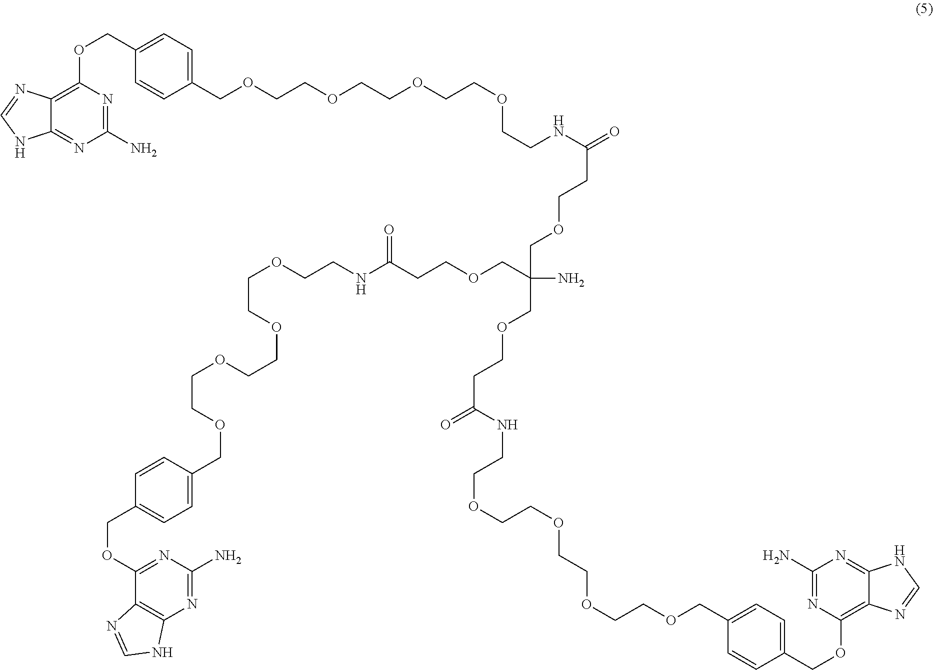

To a solution of compound (4) (1.03 g, 0.6 mmol) in ethanol (15 mL) a solution of MeNH₂ (30% in EtOH, 30 mL) is added. The corresponding solution is stirred overnight at rt. A cloudy mixture is obtained. The solid is removed by filtration and evaporation of the resulting clean solution affords the desired compound (5). No further purification is required. ESI-MS: m/z 1621.79 [M+H]⁺.

### Example 6

### N-Tris[2-(BG-PEG4-NH-carbonyl)ethyloxymethyl]methyl fluorescein-5-carboxamide (6) and corresponding fluorescein-6-carboxamide (7)

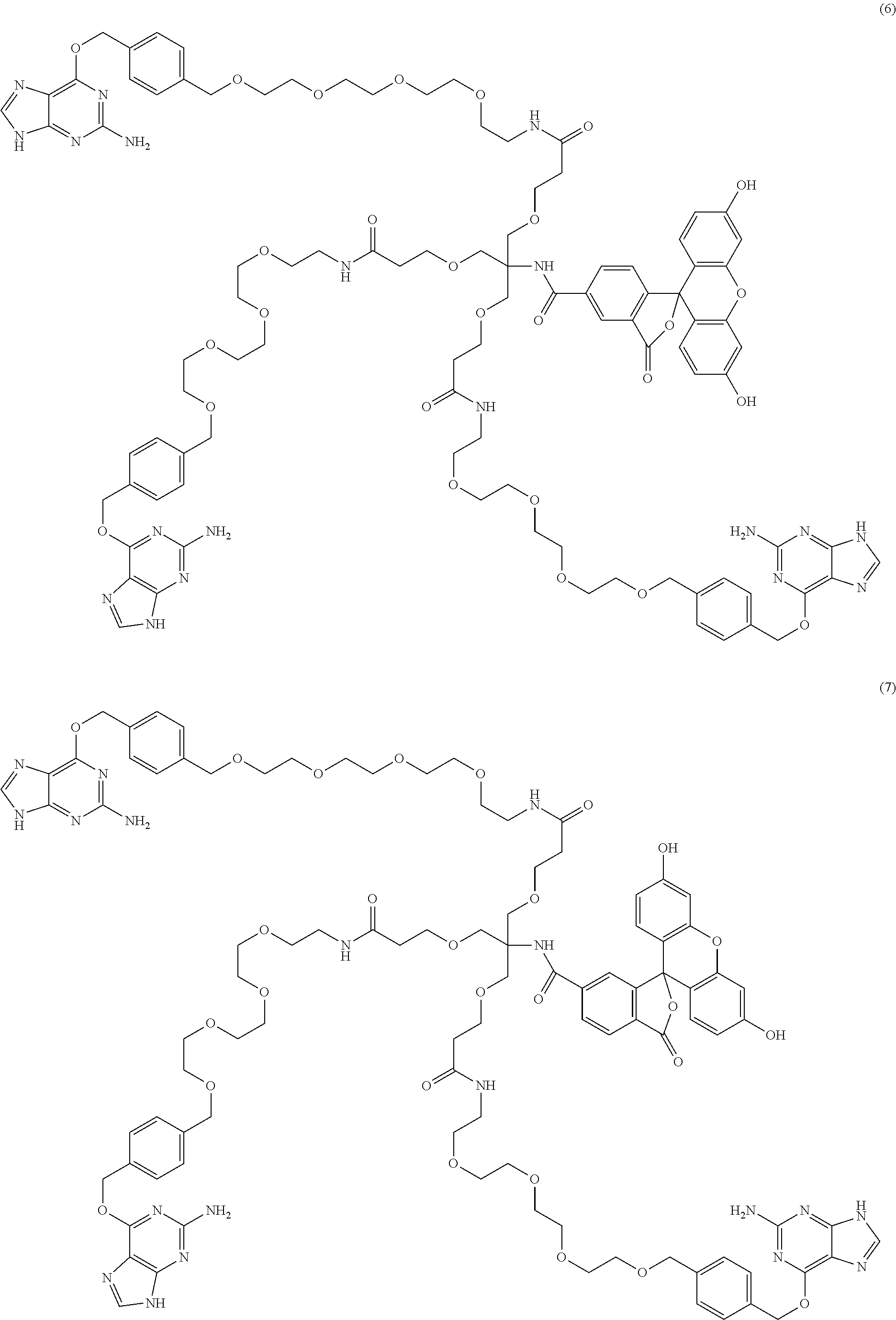

Compound (5) (29 mg, 0.018 mmol) and 5(6)-carboxyfluorescein succinimidyl ester (8.5 mg, 0.018 mmol) are dissolved in 1 mL of DMF with Et₃N (2.7 μL, 0.018 mmol) and heated overnight at 31° C. The solvent is evaporated under vacuum and compounds (6) and (7) isolated by reversed phase HPLC on a C18 column using a linear gradient of water:acetonitrile 95:5 to 20:80 in 20 min, 0.08% TFA). ESI-MS: m/z 1980.84 [M+H]⁺.

### Example 7

### N-Tris[2-(BG-PEG4-NH-carbonyl)ethyloxymethyl]methyl chlorambucil-carboxamide (8)

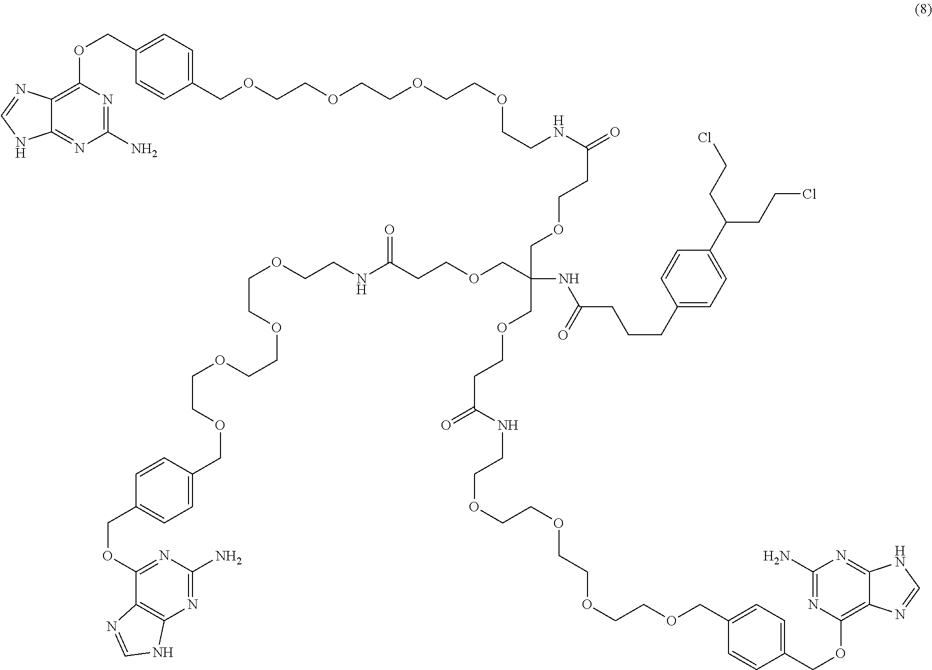

To a solution of chlorambucil (22 mg, 0.072 mmol) in DMF (2 mL) PYBOP (38 mg, 0.072 mmol) is added at rt. The solution is stirred at rt for 20 min. Compound (5) (116 mg, 0.072 mmol) and DIPEA (12 μL, 0.072 mmol) are added and the solution is heated at 50° C. for 5 min. The solution is stirred at rt overnight. Then the solvent is removed under reduced pressure and the mixture is diluted with 150 mL of ethyl acetate. The organic layer is washed with water, dried over MgSO₄ and evaporated under reduced pressure. Flash chromatography (CH₂Cl₂/MeOH, 10/1→5/1) gives the desired compound (8). ESI-MS: m/z 1906.86 [M+H]⁺.

### Example 8

### N-Tris[2-(BG-PEG4-NH-carbonyl)ethyloxymethyl]methyl 5-maleimido-pentanecarboxamide (9)

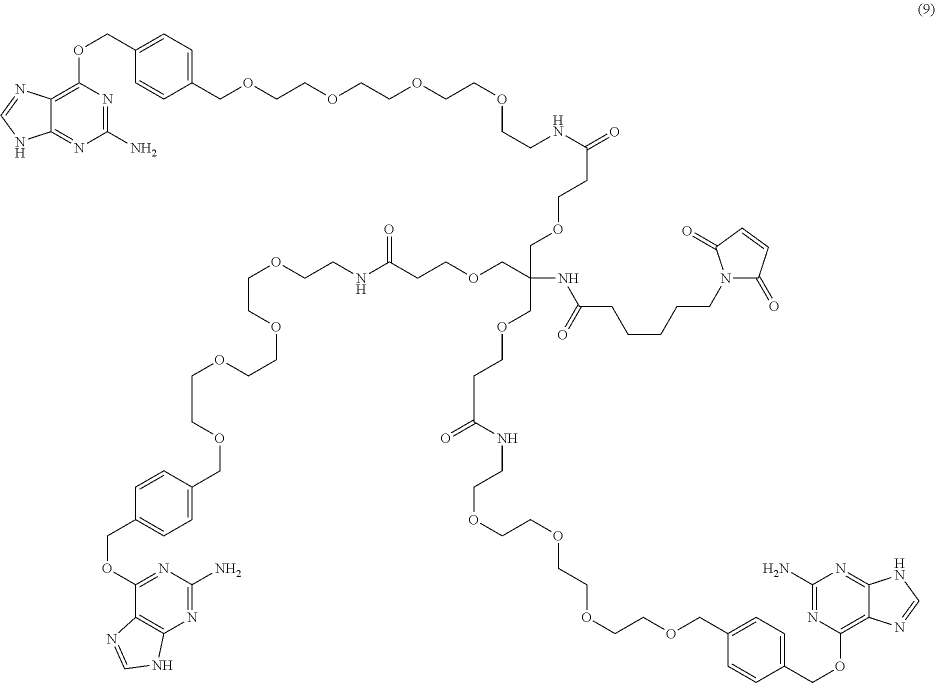

To a solution of 6-maleimido-hexanoic acid (8 mg, 0.036 mmol) in DMF (2 mL) PYBOP (19 mg, 0.036 mmol) is added at rt. The solution is stirred at rt for 20 min. Compound (5) (58 mg, 0.036 mmol) and DIPEA (6 μL, 0.036 mmol) are added and the solution is heated at 50° C. for 5 min. The solution is stirred at rt overnight. Then the solvent is removed under reduced pressure and the mixture is diluted with 150 mL of ethyl acetate. The organic layer is washed with water, dried over MgSO₄ and evaporated under reduced pressure. Flash chromatography (CH₂Cl₂/MeOH, 10/1→5/1) gives the desired compound (9). ESI-MS: m/z 1815.86 [M+H]⁺.

### Example 9

### Tris[2-(BG-PEG4-NH-carbonyl)ethyloxymethyl]methyl 6-(2-thiosuccinimido)-hexanoic amide siRNA conjugate (10)

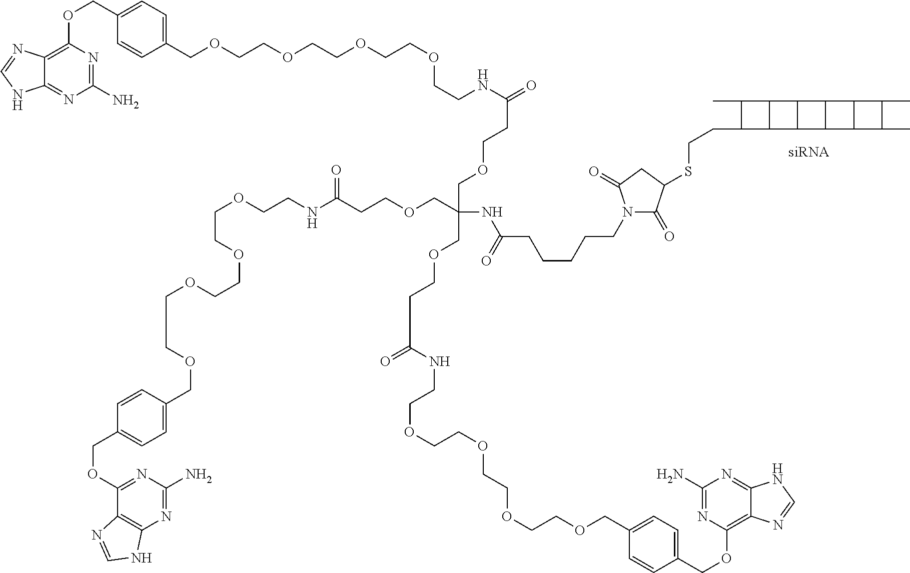

5′-Thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL of a solution of compound (9) (2.5 mM in DMF) is added and the reaction mixture incubated at room temperature for 1 h. The reaction mixture is diluted with water to a total volume of 2 mL and excess maleimide removed by gel filtration. The tris[2-(BG-PEG4-NH-carbonyl)ethyloxymethyl]methylamide oligonucleotide-thiosuccinimide conjugate (10) is then purified by HPLC (solvent A: 0.1 M tetraethylammonium acetate pH 6.9 in water; solvent B: acetonitrile).

### Example 10

### N-2-{2-[2-(2-azidoethoxy)ethoxy]ethoxy}ethyl-N′-tris[2-(tert-butoxy-carbonyl)ethyl]methyl-urea (11)

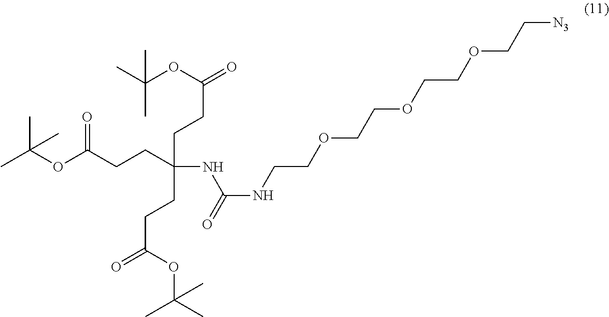

To a solution of 11-azido-3,6,9-trioxaundecan-1-amine (1.55 g, 1 eq, 7.1 mmol) in DMF (2 mL) tris[2-(tert-butoxycarbonyl)ethyl]methylisocyanate (3.1 g, 1 eq, 7.1 mmol) and Et₃N (988 μL, 1 eq, 7.1 mmol) are added. The solution is stirred overnight at 31° C. The crude mixture is poured into water and extracted with ethyl acetate, the organic phase is dried over MgSO₄, and evaporated under reduced pressure to afford (11). No further purification is required. FAB-MS: m/z 660.41 [M+H]⁺.

### Example 11

### 4-[2-carboxyethyl]-4-{2-[2-(2-(2-azidoethoxy)ethoxy)ethoxy]ethylamino-carbonylamino}-1,7-heptanedicarboxylic acid (12)

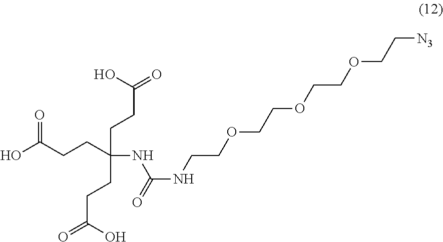

Compound (11) (3.3 g, 5 mmol) is stirred in 50 mL of 96% formic acid for 18 h. The formic acid is removed under reduced pressure at 50° C. to produce a colorless oil, compound (12). ESI-MS: m/z 492.22 [M+H]⁺.

### Example 12

### N-Tris[2-(BG-PEG4-NH-carbonyl)ethyl]methyl-N′-2-{2-[2-(2-azidoethoxy)ethoxy]ethoxy}ethyl-urea (13)

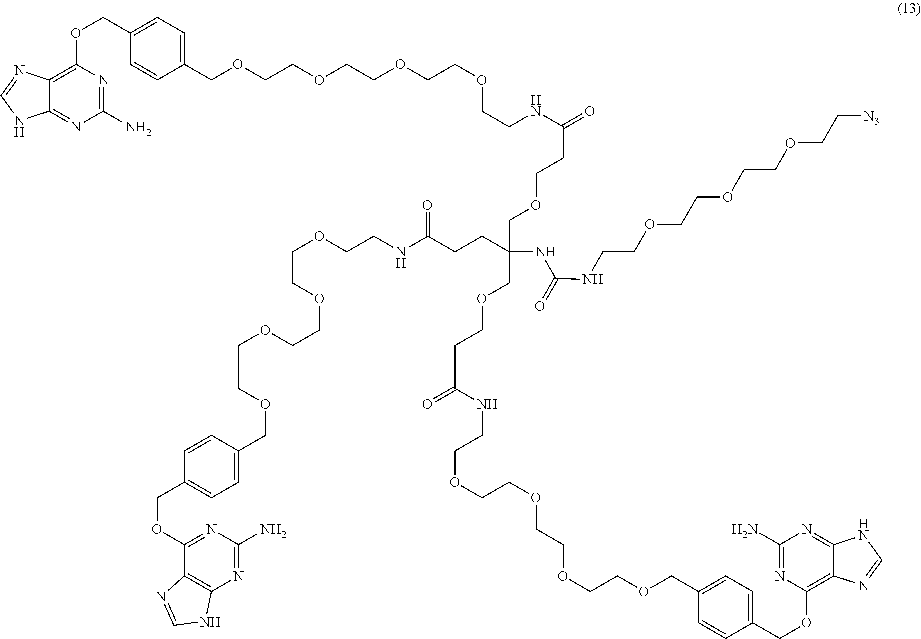

To a solution of compound (12) (491 mg, 1 mmol, 1 eq) and BG-PEG4-NH2 (1.34 g, 3 mmol, 3 eq) in DMF (50 mL) are successively added DIPEA (495 μL, 3 mmol, 3 eq), HOBT (1 M in NMP, 3 mL, 3 mmol, 3 eq) and DCC (620 mg, 3 mmol, 3 eq) at rt. The resulting mixture is stirred overnight. The solvent is removed under reduced pressure and the mixture is diluted with 250 mL of ethyl acetate. The organic layer is washed with water, dried over MgSO₄ and evaporated under reduced pressure. Flash chromatography (CH₂Cl₂/methanol, 10/1→5/1) gives the desired compound (13). ESI-MS: m/z 1776.87 [M+H]⁺.

### Example 13

### N-Tris[2-(BG-PEG4-NH-carbonyl)ethyl]methyl-N′-2-{2-[2-(2-aminoethoxy)ethoxy]ethoxy}ethyl-urea (14)

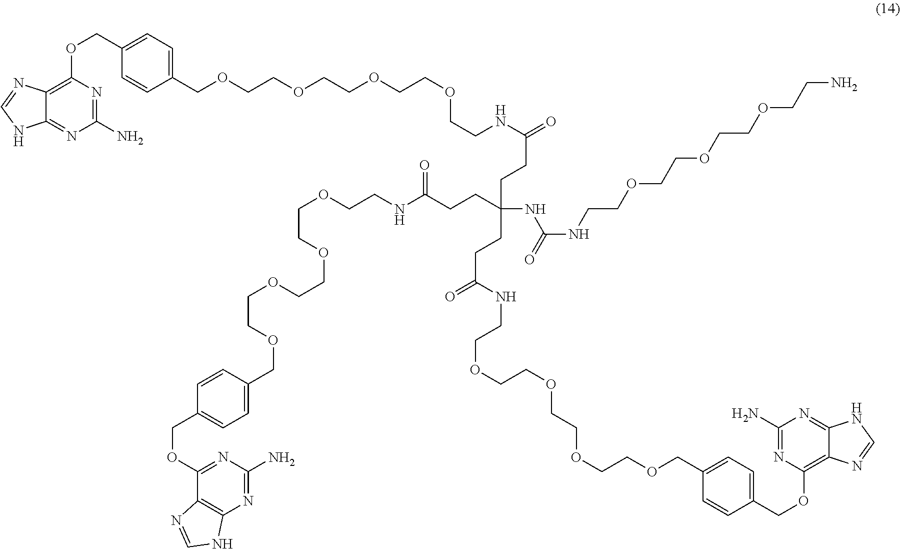

To a solution of compound (13) (708 mg, 0.4 mmol) in dioxane (10 mL) water (1 mL) is added. PMe₃ (2.40 mL 1 M in THF solution, 6 eq) is added and the solution is stirred at rt for 2 h. The solvent is removed under reduced pressure, and compound (14) is obtained by purification with preparative HPLC. ESI-MS: m/z 1750.88 [M+H]⁺.

### Example 14

### N-Tris[2-(BG-PEG4-NH-carbonyl)ethyl]methyl-N′-2-{2-[2-(2-fluorescein-5-carboxamido-ethoxy)ethoxy]ethoxy}ethyl-urea (15) and corresponding 6-fluorescein derivative (16)

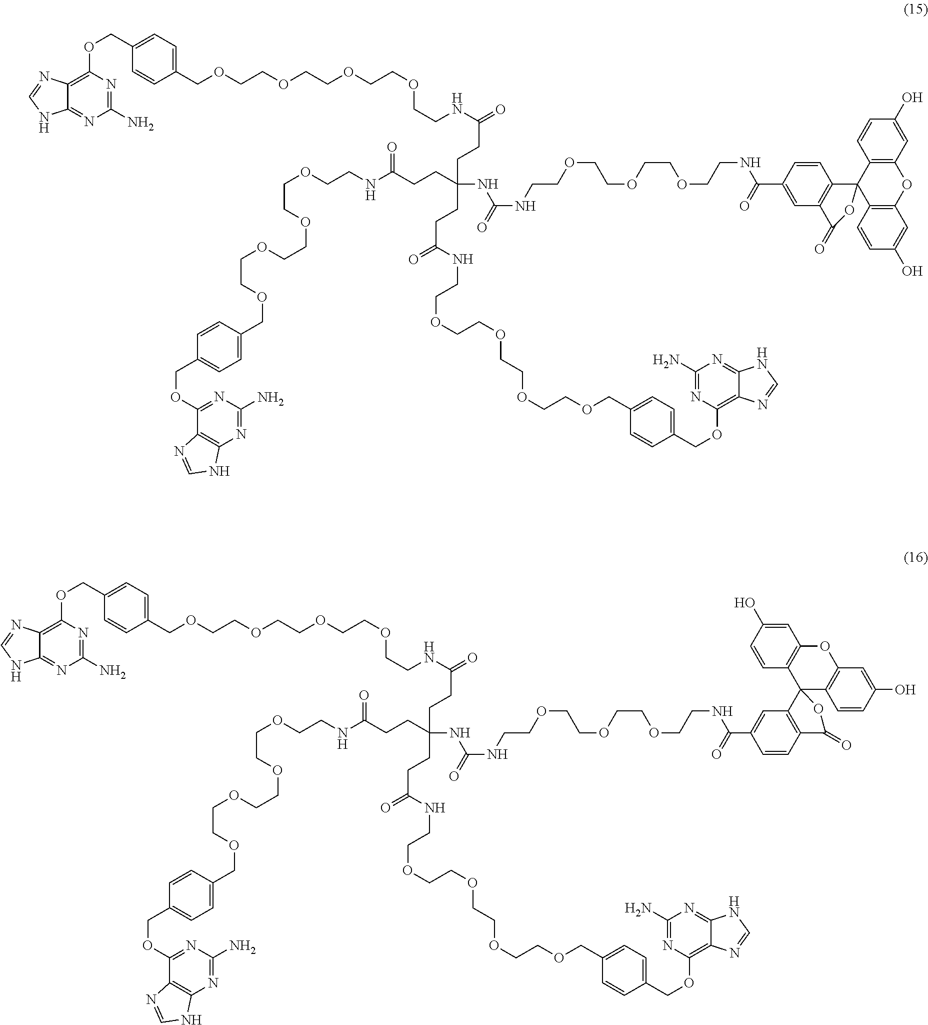

Compound (14) (18 mg, 0.01 mmol) and 5(6)-carboxyfluorescein succinimidyl ester (5 mg, 0.01 mmol) are dissolved in 800 μL DMF with Et₃N (1.6 μL, 0.01 mmol) and heated overnight at 31° C. The solvent is evaporated under vacuum and compounds (15) and (16) isolated by reversed phase HPLC on a C18 column using a linear gradient of water:acetonitrile (from 95:5 to 20:80 in 20 min, 0.08% TFA).

ESI-MS: m/z 2108.93 [M+H]⁺.

### Example 15

### N-Tris[2-(BG-PEG4-NH-carbonyl)ethyl]methyl-N′-2-{2-[2-(2-chlorambucil-carboxamido-ethoxy)ethoxy]ethoxy}ethyl-urea (17)

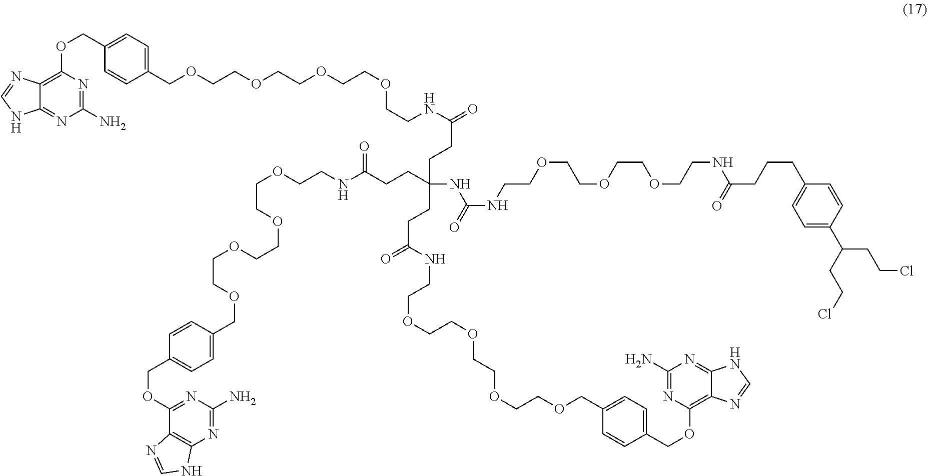

To a solution of chlorambucil (18 mg, 0.06 mmol) in DMF (3 mL) PYBOP (31 mg, 0.06 mmol) is added at rt. The solution is stirred at rt for 20 min. Compound (14) (103 mg, 0.06 mmol) and DIPEA (10 μL, 0.06 mmol) are added and the solution is heated at 50° C. for 5 min. The solution is stirred at rt overnight. The solvent is removed under reduced pressure and the mixture is diluted with 150 mL of ethyl acetate. The organic layer is washed with water, dried over MgSO₄ and evaporated under reduced pressure. Flash chromatography (CH₂Cl₂/MeOH, 10/1→5/1) gives the desired compound (17).

ESI-MS: m/z 2050.99 [M+H]⁺.

### Example 16

### N-Tris[2-(BG-PEG4-NH-carbonyl)ethyl]methyl-N′-2-{2-[2-(2-(6-maleimido-hexanoylamido)ethoxy)ethoxy]ethoxy}ethyl-urea (181

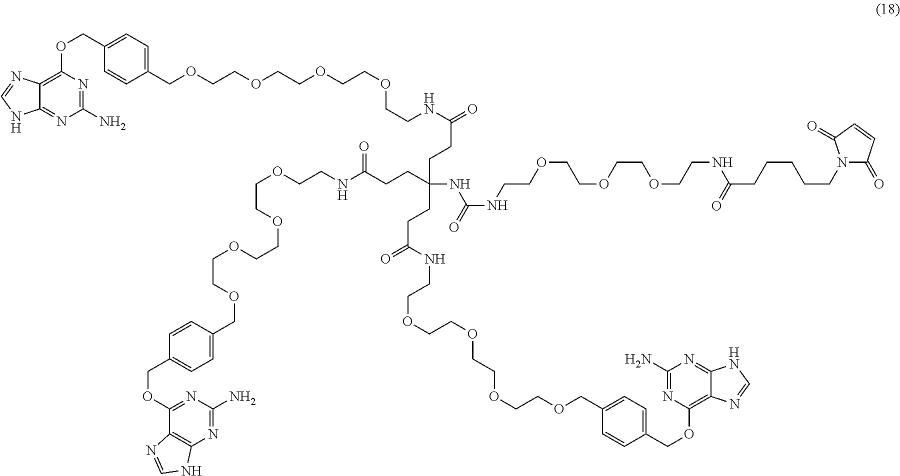

To a solution of 6-maleimidohexanoic acid (10 mg, 0.046 mmol) in DMF (2 mL) PYBOP (24 mg, 0.046 mmol) is added at rt. The solution is stirred at rt for 20 min. Compound (14) (80 mg, 0.046 mmol) and DIPEA (7.7 μL, 0.046 mmol) are added and the solution is heated at 50° C. for 5 min. The solution is stirred at rt overnight. The solvent is removed under reduced pressure and the mixture is diluted with 150 mL of ethyl acetate. The organic layer is washed with water, dried over MgSO₄ and evaporated under reduced pressure. Flash chromatography (CH₂Cl₂/MeOH, 10/1→5/1) gives the desired compound (18). ESI-MS: m/z 1959.99 [M+H]⁺.

### Example 17

### N-Tris[2-(BG-PEG4-NH-carbonyl)ethyl]methyl-N′-2-{2-[2-(2-{6-[2-thio-succinimido]hexanoylamido}ethoxy)ethoxy]ethoxy}ethyl-urea siRNA conjugate (19)

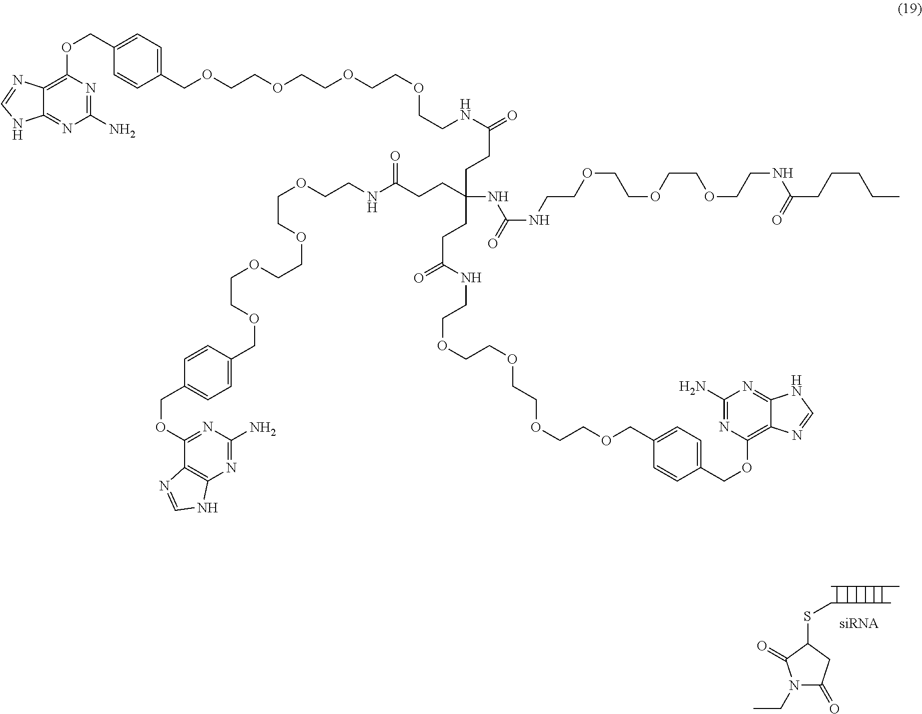

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL of a solution of compound (18) (2.5 mM in DMF) is added and the reaction mixture incubated at room temperature for 1 h. The reaction mixture is diluted with water to a total volume of 2 mL and excess maleimide removed by gel filtration. The siRNA conjugate (19) is then purified by HPLC (solvent A: 0.1 M tetraethylammonium acetate pH 6.9 in water; solvent B: acetonitrile).

### Example 18

### Azido-PEG12-propionic acid 2-maleimidoethylamide (20)

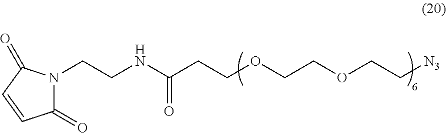

N-(2-Aminoethyl)maleimide trifluoroacetate (343 mg, 1.35 mmol) and azido-PEG12-propionic NHS ester (1 g, 1.35 mmol) are dissolved in 5 mL DMF with Et₃N (188 μL, 1.35 mmol) and heated overnight at 31° C. The solvent is evaporated under vacuum and the product is isolated by reversed phase HPLC on a C18 column using a linear gradient of water:acetonitrile (from 95:5 to 20:80 in 20 min, 0.08% TFA).

ESI-MS: m/z 766.40 [M+H]⁺.

### Example 19

### Azido-PEG12-propionic acid 2-maleimidoethylamide CoA-SH conjugate (21)

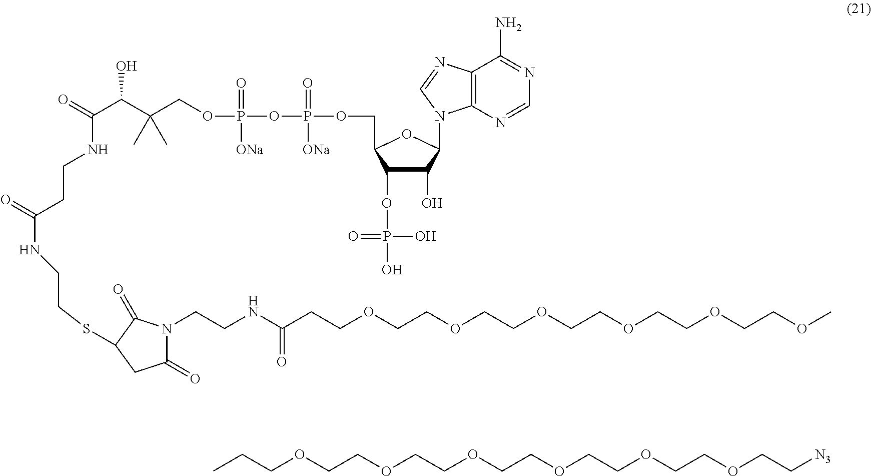

A solution of maleimide derivative (20) (192 mg, 1 eq, 252 μmol) in DMF (2 mL) is added to a solution of CoA-SH (248 mg, 1.2 eq, 304 μmol) in Tris-buffer (pH 7.5, 200 μL). The reaction mixture is shaken overnight at 31° C. The solvent is removed under vacuum and the crude mixture is purified via preparative HPLC. ESI-MS: m/z 1554.48 [M-Na]⁻.

### Example 20

### Amino-PEG12-propionic acid 2-maleimidoethylamide CoA-SH conjugate (22)

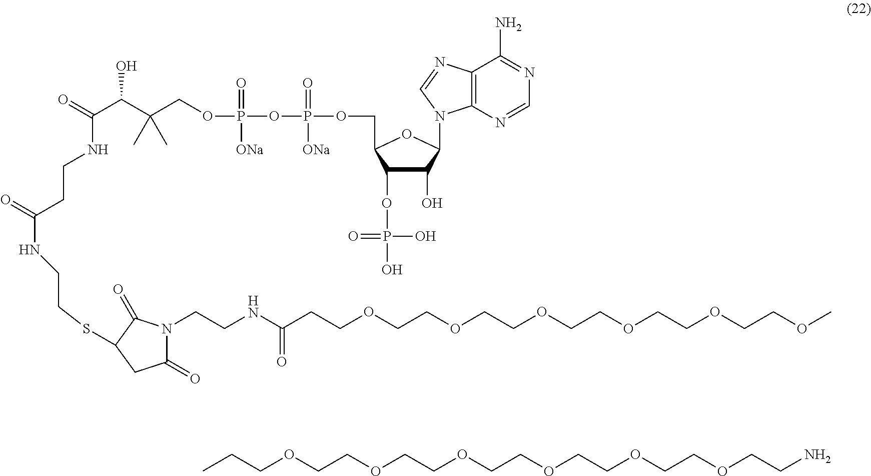

To a solution of compound (21) (204 mg, 0.13 mmol) in dioxane (3 mL) water (450 μL) is added. PMe₃ (800 μL 1 M in THF solution, 6 eq) is added and the solution is stirred at rt for 2 h. The solvent is removed under reduced pressure, and the compound is obtained by purification with preparative HPLC. ESI-MS: m/z 1527.48 [M-Na]⁻.

### Example 21

### N-Tris{2-[2-(2-(CoA-S-succinimido)ethylaminocarbonyl-PEG12)ethyl-aminocarbonyl]ethoxymethyl}methyl trifluoroacetamide (23)

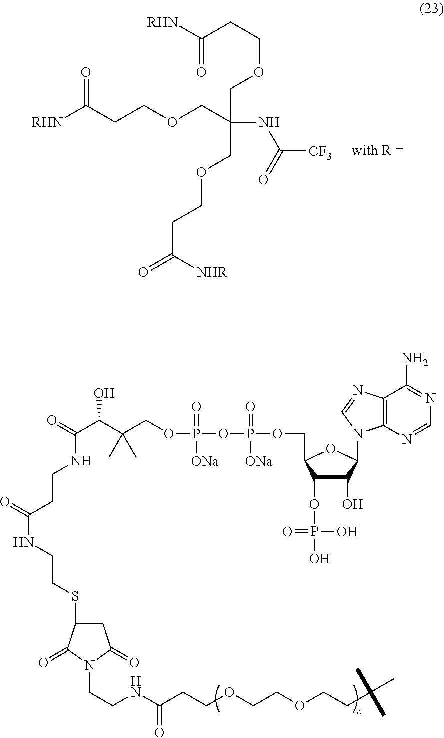

To a solution of (3) (21 mg, 0.05 mmol, 1 eq) and (22) (232 mg, 0.15 mmol, 3 eq) in DMF (1 mL) are successively added DIPEA (25 μL, 0.15 mmol, 3 eq), HOBT (1 M in NMP, 150 μL, 0.3 mmol, 3 eq) and DCC (31 mg, 015 mmol, 3 eq) at rt. The resulting mixture is stirred overnight. The solvent is removed under reduced pressure, and compound (23) is obtained by purification with preparative HPLC. ESI-MS: m/z 5010.4 [M-Na]⁻.

### Example 22

### N-Tris{2-[2-(2-(CoA-S-succinimido)ethylaminocarbonyl-PEG12)ethyl-aminocarbonyl]ethoxymethyl}methylamine (24)

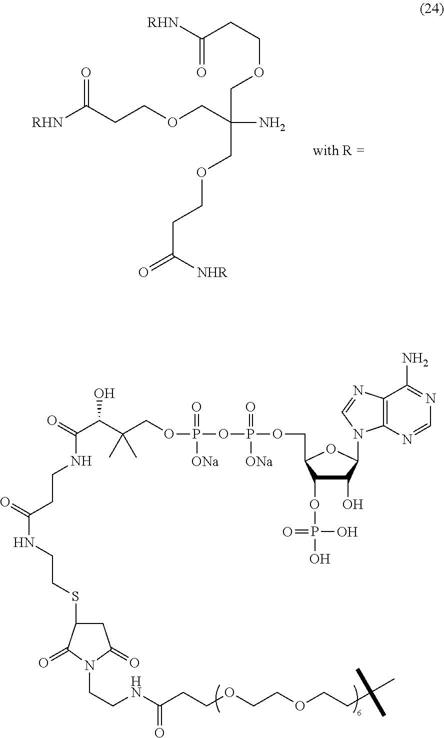

To a solution of compound (21) (100 mg, 0.02 mmol) in ethanol (1.5 mL) a solution of MeNH₂ (3 mL, 30% in EtOH) is added. The solution is stirred overnight at rt. Evaporation of the solvent affords the desired compound (24). No further purification is required.

ESI-MS: m/z 4914.4 [M-Na]⁻.

### Example 23

### N-Tris{2-[2-(2-(CoA-S-succinimido)ethylaminocarbonyl-PEG12)ethyl-aminocarbonyl]ethoxymethyl}methyl fluorescein-5-carboxamide (25) and corresponding fluorescein-6-carboxamide (26)

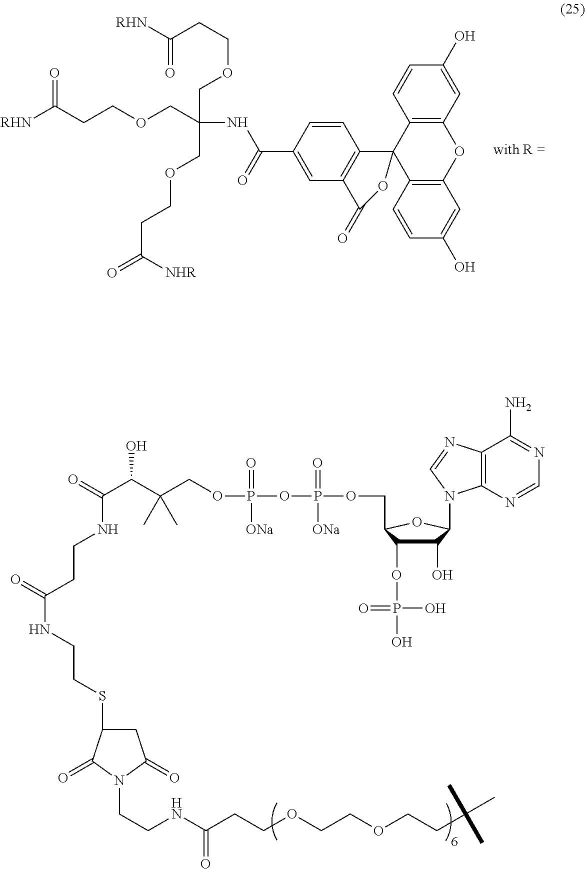

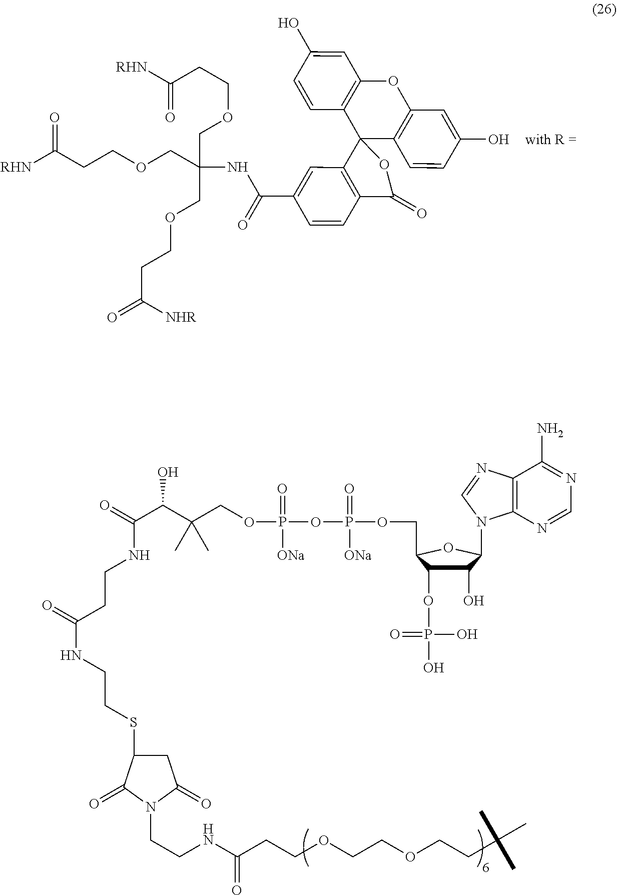

Compound (24) (19 mg, 0.004 mmol) and 5(6)-carboxyfluorescein NHS ester (2 mg, 0.004 mmol) are dissolved in 600 μL DMF with Et₃N (0.6 μL, 0.004 mmol) and heated overnight at 31° C. The solvent is evaporated under vacuum and the compounds (25) and (26) isolated by reversed phase HPLC on a C18 column using a linear gradient of water:acetonitrile (from 95:5 to 20:80 in 20 min, 0.08% TFA).

ESI-MS: m/z 5272.7 [M-Na]⁻.

### Example 24

### N-Tris{2-[2-(2-(CoA-S-succinimido)ethylaminocarbonyl-PEG12)ethyl-aminocarbonyl]ethoxymethyl}methyl chlorambucil-carboxamide (27)

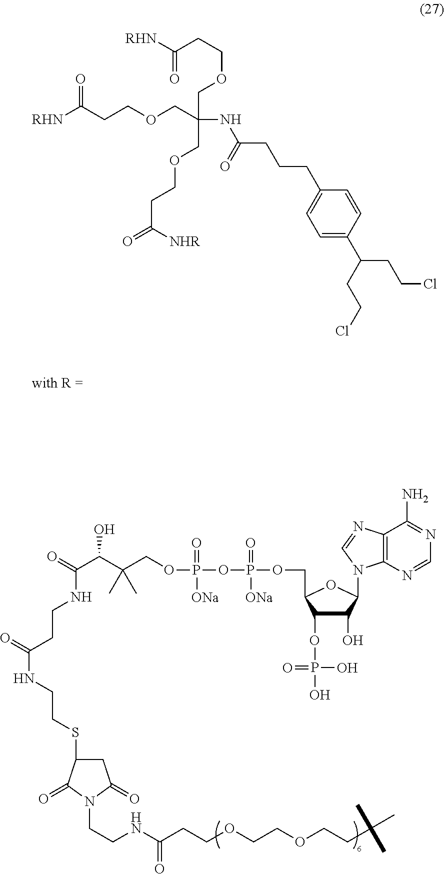

To a solution of chlorambucil (1.8 mg, 0.006 mmol) in DMF (1 mL) PYBOP (3 mg, 0.006 mmol) is added at rt. The solution is stirred at rt for 20 min. Compound (24) (29 mg, 0.006 mmol) and DIPEA (0.9 μL, 0.006 mmol) are added and the solution is heated at 50° C. for 5 min. The solution is stirred at rt overnight. The solvent is removed under reduced pressure. Compound (27) is isolated by reversed phase HPLC on a C18 column using a linear gradient of water:acetonitrile (from 95:5 to 20:80 in 20 min, 0.08% TFA).

ESI-MS: m/z 5200.6 [M-Na]⁻.

### Example 25

### N-Tris{2-[2-(2-(CoA-S-succinimido)ethylaminocarbonyl-PEG12)ethyl-aminocarbonyl]ethoxymethyl}methyl 6-maleimidohexanoylamide (28)

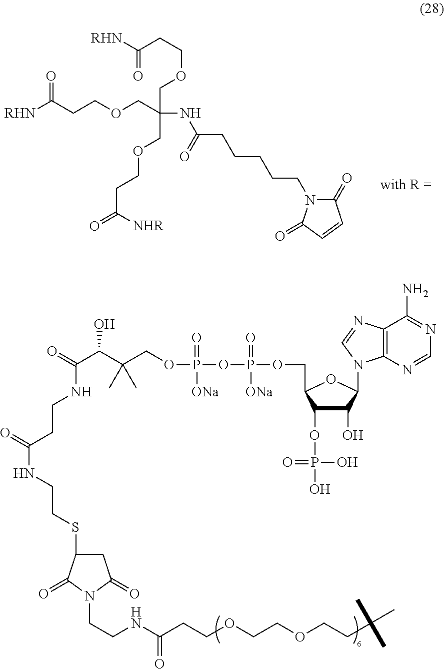

To a solution of 6-maleimido-hexanoic acid (0.844 mg, 0.004 mmol) in DMF (1 mL) PYBOP (2.08 mg, 0.004 mmol) is added at rt. The solution is stirred at rt for 20 min. Compound (24) (19 mg, 0.004 mmol) and DIPEA (0.6 6 μL, 0.004 mmol) are added and the solution is heated at 50° C. for 5 min. The solution is stirred at rt overnight. The solvent is removed under reduced pressure. Compound (28) is isolated by reversed phase HPLC on a C18 column using a linear gradient of water:acetonitrile (from 95:5 to 20:80 in 20 min, 0.08% TFA). ESI-MS: m/z 5107.7 [M-Na]⁻.

### Example 26

### N-Tris{2-[2-(2-(CoA-S-succinimido)ethylaminocarbonyl-PEG12)ethyl-aminocarbonyl]ethoxymethyl}methyl 6-(2-thiosuccinimido)hexanoylamide siRNA conjugate (29)

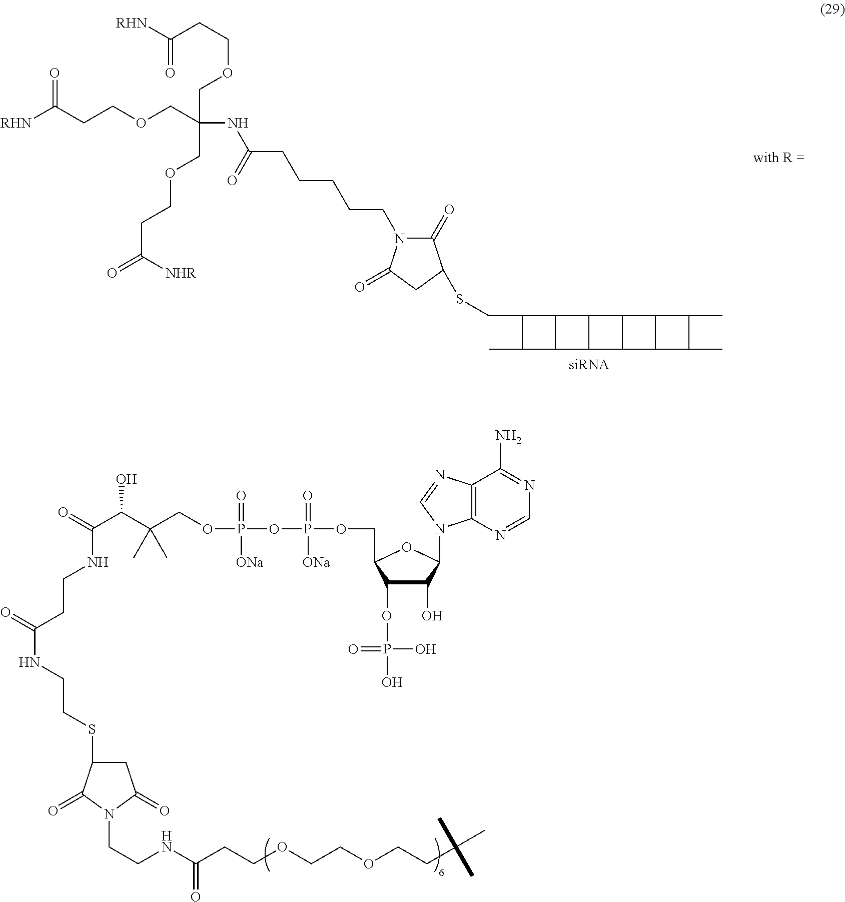

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL of a solution of compound (28) (2.5 mM in DMF) is added and the reaction mixture incubated at room temperature for 1 h. The reaction mixture is diluted with water to a total volume of 2 mL and excess maleimide removed by gel filtration. The conjugate (29) is then purified by HPLC (solvent A: 0.1 M tetraethyl-ammonium acetate pH 6.9 in water; solvent B: acetonitrile).

### Example 27

### N-Tris{2-[2-(2-(CoA-S-succinimido)ethylaminocarbonyl-PEG12)ethyl-aminocarbonyl]ethoxymethyl}methyl-N′-2-{2-[2-(2-azidoethoxy)ethoxy]-ethoxy}ethyl-urea (30)

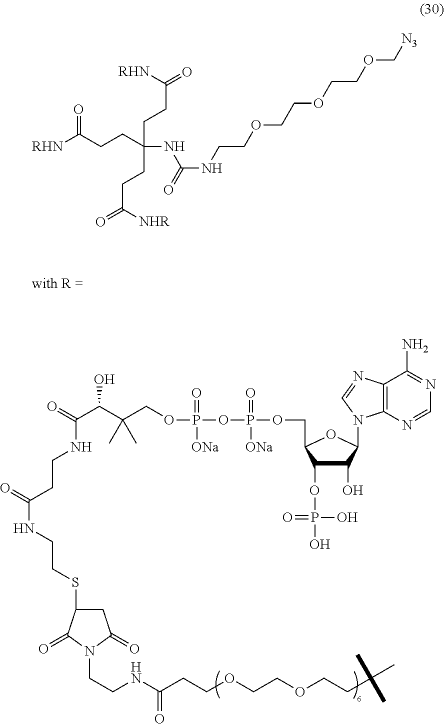

To a solution of compound (12) (49 mg, 0.1 mmol, 1 eq) and compound (22) (134 mg, 0.3 mmol, 3 eq) in DMF (5 mL) are successively added DIPEA (49 μL, 0.3 mmol, 3 eq), HOBT (1 M in NMP, 0.3 mL, 0.3 mmol, 3 eq) and DCC (62 mg, 0.3 mmol, 3 eq) at rt. The resulting mixture is stirred overnight. The solvent is removed under reduced pressure. Compound (30) is isolated by reversed phase HPLC on a C18 column using a linear gradient of water:acetonitrile (from 95:5 to 20:80 in 20 min, 0.08% TFA).

ESI-MS: m/z 5068.6 [M-Na]⁻.

### Example 28

### N-Tris{2-[2-(2-(CoA-S-succinimido)ethylaminocarbonyl-PEG12)ethyl-aminocarbonyl]ethoxymethyl}methyl-N′-2-{2-[2-(2-aminoethoxy)ethoxy]-ethoxy}ethyl-urea (31)

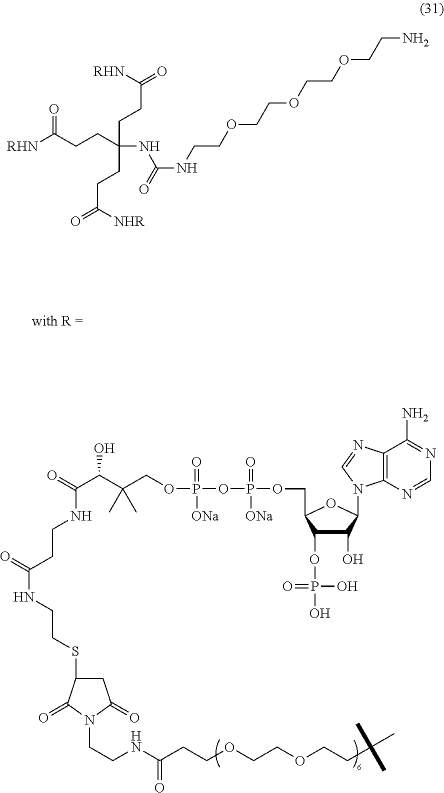

To a solution of compound (30) (127 mg, 0.025 mmol) in dioxane (3 mL) water (450 μL) is added. PMe₃(154 μL of 1 M THF solution, 6 eq) is added and the solution is stirred at rt for 2 h. The solvent is removed under reduced pressure, and compound (31) is obtained by purification with preparative HPLC on a C18 column using a linear gradient of water:acetonitrile (from 95:5 to 20:80 in 20 min, 0.08% TFA).

ESI-MS: m/z 5042.5 [M-Na]⁻.

### Example 29

### N-Tris{2-[2-(2-(CoA-S-succinimido)ethylaminocarbonyl-PEG12)ethyl-aminocarbonyl]ethoxymethyl}methyl-N′-2-{2-[2-(2-fluorescein-5-carboxamidoethoxy)ethoxy]ethoxy}ethyl-urea (32) and corresponding 6-fluorescein derivative (33)

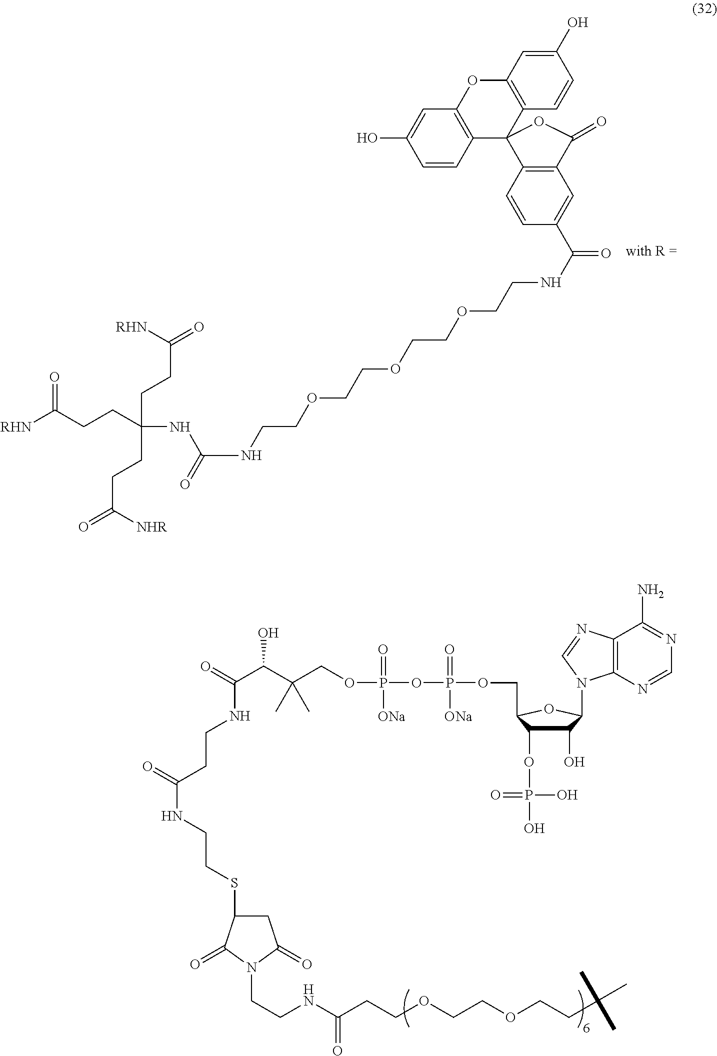

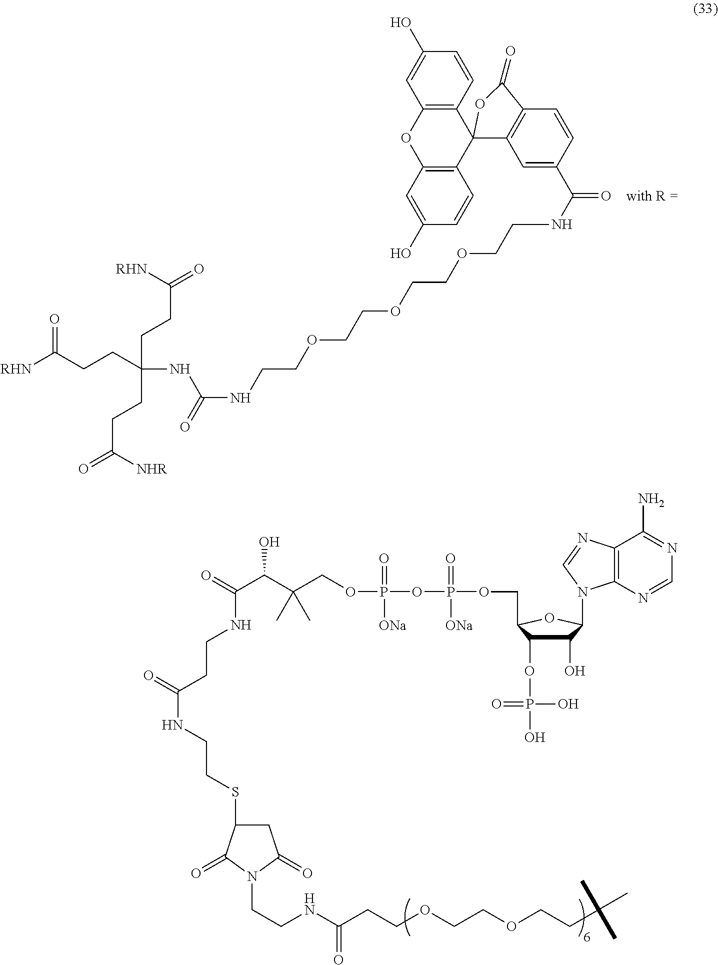

Compound (31) (20 mg, 0.004 mmol) and 5(6)-carboxyfluorescein NHS ester (2 mg, 0.004 mmol) are dissolved in 600 μL DMF with Et₃N (0.6 μL, 0.004 mmol) and heated overnight at 31° C. The solvent is evaporated under vacuum and compounds (32) and (33) isolated by reversed phase HPLC on a C18 column using a linear gradient of water:acetonitrile (from 95:5 to 20:80 in 20 min, 0.08% TFA).

ESI-MS: m/z 5400.9 [M-Na]⁻.

### Example 30

### N-Tris{2-[2-(2-(CoA-S-succinimido)ethylaminocarbonyl-PEG12)ethyl-aminocarbonyl]ethoxymethyl}methyl-N′-2-{2-[2-(2-chlorambucil-carboxamido-ethoxy)ethoxy]ethoxy}ethyl-urea (34)

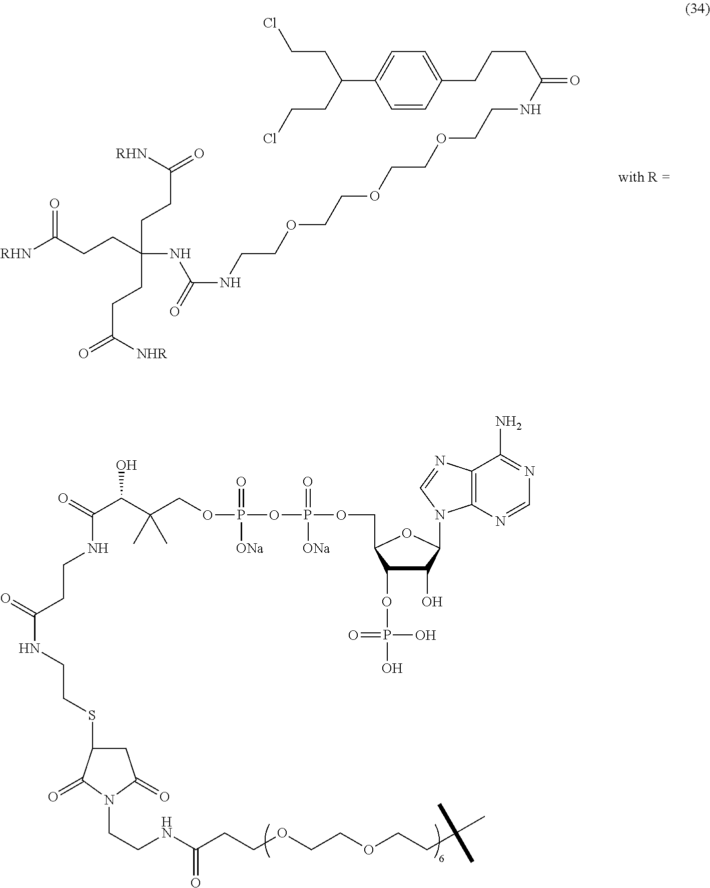

To a solution of chlorambucil (2.1 mg, 0.007 mmol) in DMF (1 mL) PYBOP (3.5 mg, 0.007 mmol) is added at rt. The solution is stirred at rt for 20 min. Compound (31) (35 mg, 0.007 mmol) and DIPEA (1.1 μL, 0.007 mmol) are added and the solution is heated at 50° C. for 5 min. The solution is stirred at rt overnight. Then the solvent is removed under reduced pressure. Compound (34) is isolated by reversed phase HPLC on a C18 column using a linear gradient of water:acetonitrile (from 95:5 to 20:80 in 20 min, 0.08% TFA).

ESI-MS: m/z 5327.8 [M-Na]⁻.

### Example 31

### N-Tris-{2-[2-(2-(CoA-S-succinimido)ethylaminocarbonyl-PEG12)ethyl-aminocarbonyl]ethoxymethyl}methyl-N′-2-{2-[2-(2-(6-maleimidohexanoyl-amido)ethoxy)ethoxy]ethoxy}ethyl-urea (35)

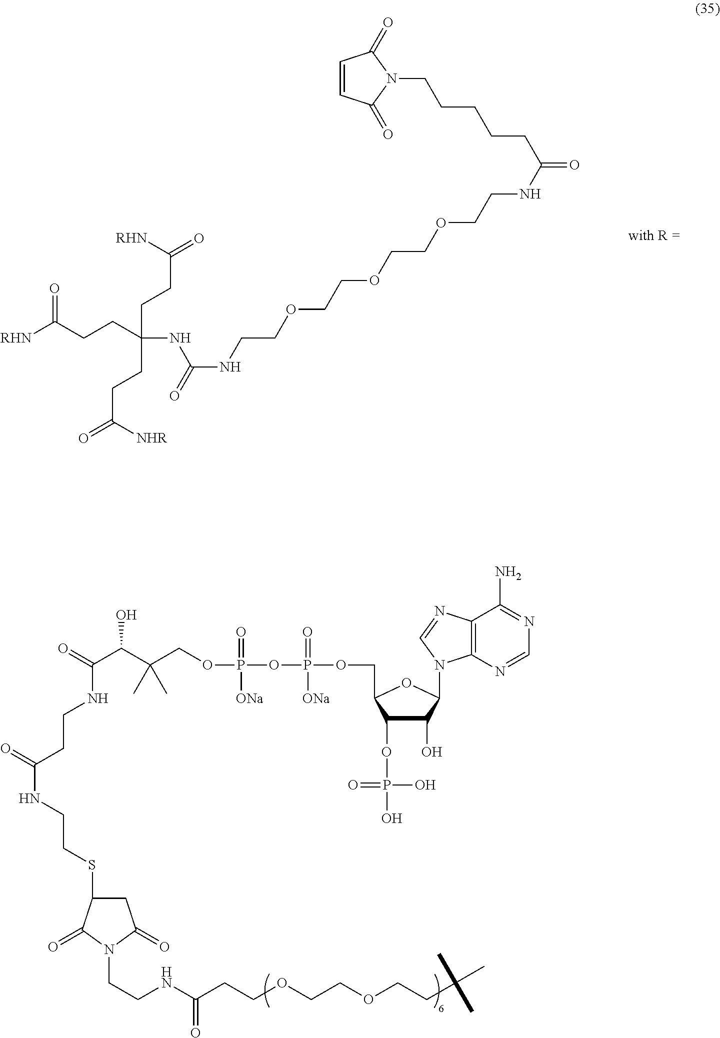

To a solution of 6-maleimido-hexanoic acid (1 mg, 0.005 mmol) in DMF (1 mL) PYBOP (2.5 mg, 0.005 mmol) is added at rt. The solution is stirred at rt for 20 min. Compound (31) (24 mg, 0.005 mmol) and DIPEA (0.8 μL, 0.005 mmol) are added and the solution is heated at 50° C. for 5 min. The solution is stirred at rt overnight. The solvent is removed under reduced pressure. Compound (35) is isolated by reversed phase HPLC on a C18 column using a linear gradient of water:acetonitrile (from 95:5 to 20:80 in 20 min, 0.08% TFA). ESI-MS: m/z 5235.7 [M-Na]⁻.

### Example 32

### N-Tris{2-[2-(2-(CoA-S-succinimido)ethylaminocarbonyl-PEG12)ethyl-aminocarbonyl]ethoxymethyl}methyl-N′-2-{2-[2-(2-(6-maleimidohexano amido)ethoxy)ethoxy]ethoxy}ethyl-urea siRNA conjugate (36)

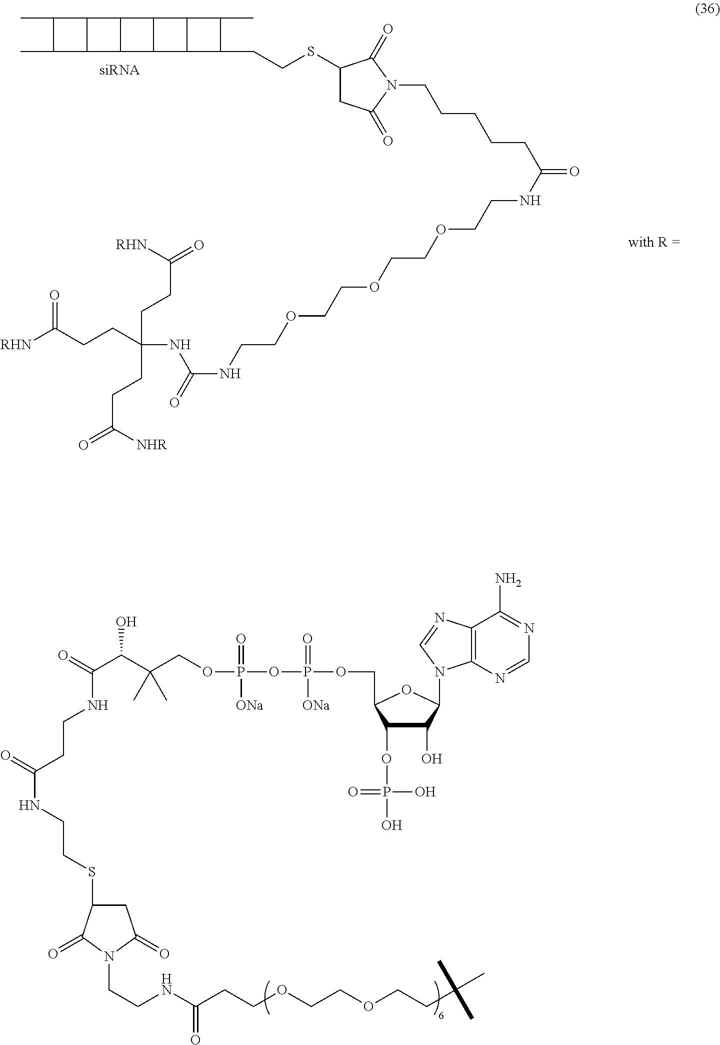

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL of a solution of compound (35) (2.5 mM in DMF) is added and the reaction mixture incubated at room temperature for 1 h. The reaction mixture is diluted with water to a total volume of 2 mL, and excess maleimide removed by gel filtration. Conjugate (36) is then purified by HPLC (solvent A: 0.1 M tetraethylammonium acetate pH 6.9 in water; solvent B: acetonitrile).

### Example 33

### 5-Fluorescein-Lys-Fmoc-OH (37) and 6-fluorescein-Lvs-Fmoc-OH (38)

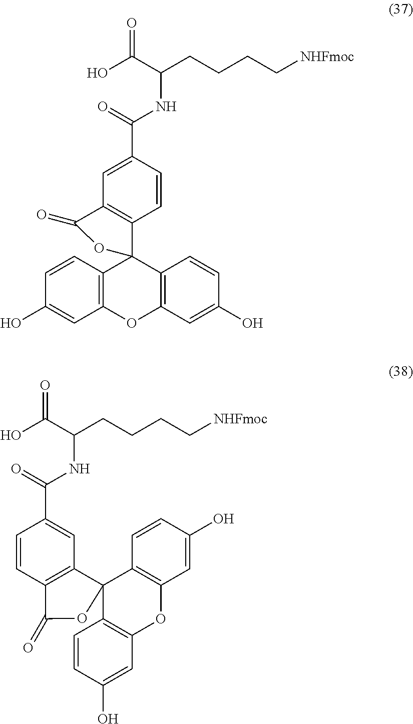

Fmoc-Lys-OH (184 mg, 0.5 mmol) and 5(6)-carboxyfluorescein NHS ester (237 mg, 0.5 mmol) are dissolved in 5 mL of DMF with Et₃N (70 μL, 0.5 mmol) and heated overnight at 31° C. The crude mixture is poured into water (100 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (37) and (38). ESI-MS: m/z 727.7 [M+H]⁺.

### Example 34

### 5-Fluorescein-Lys-OH (39) and 6-fluorescein-Lys-OH (40)

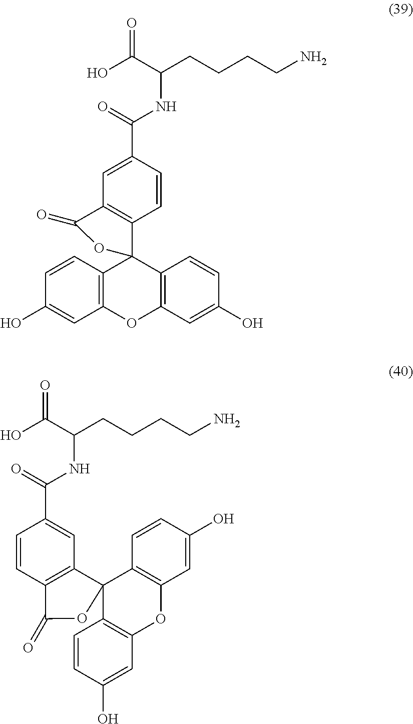

To a solution of the mixture of compounds (37) and (38) (300 mg, 0.4 mmol) in DMF (3 mL) diethylamine (600 μL) is added at rt. The solution is stirred at rt for 3 h. The solvent is removed under reduced pressure and the desired mixture of compounds (39) and (40) is directly used for the next step. ESI-MS: m/z 505.15 [M+H]⁺.

### Example 35

### N-5-Fluorescein-N′-chlorambucil-Lys-OH (41) and N-6-fluorescein-N′-chlorambucil-Lys-OH (42)

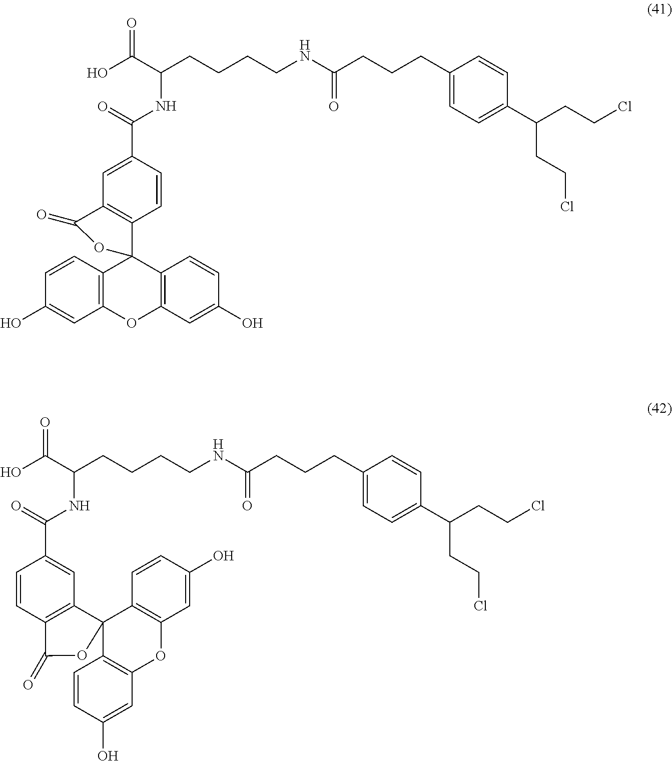

To a solution of chlorambucil (106 mg, 0.35 mmol) in DMF (3 mL) PYBOP (182 mg, 0.35 mmol) is added at rt. The solution is stirred at rt for 20 min. The mixture of isomers (39) and (40) (176 mg, 0.35 mmol) and DIPEA (58 μL, 0.35 mmol) are added and the solution is heated at 50° C. for 5 min. The solution is stirred at rt overnight. The crude mixture is poured into water (60 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (41) and (42).

ESI-MS: m/z 789.23 [M+H]⁺.

### Example 36

### N-Tris[2-(BG-PEG4-NH-carbonyl)ethyloxymethyl]methyl N′-5-fluorescein-N″-chlorambucil-Lys-amide (43) and corresponding 6-fluorescein derivative (44)

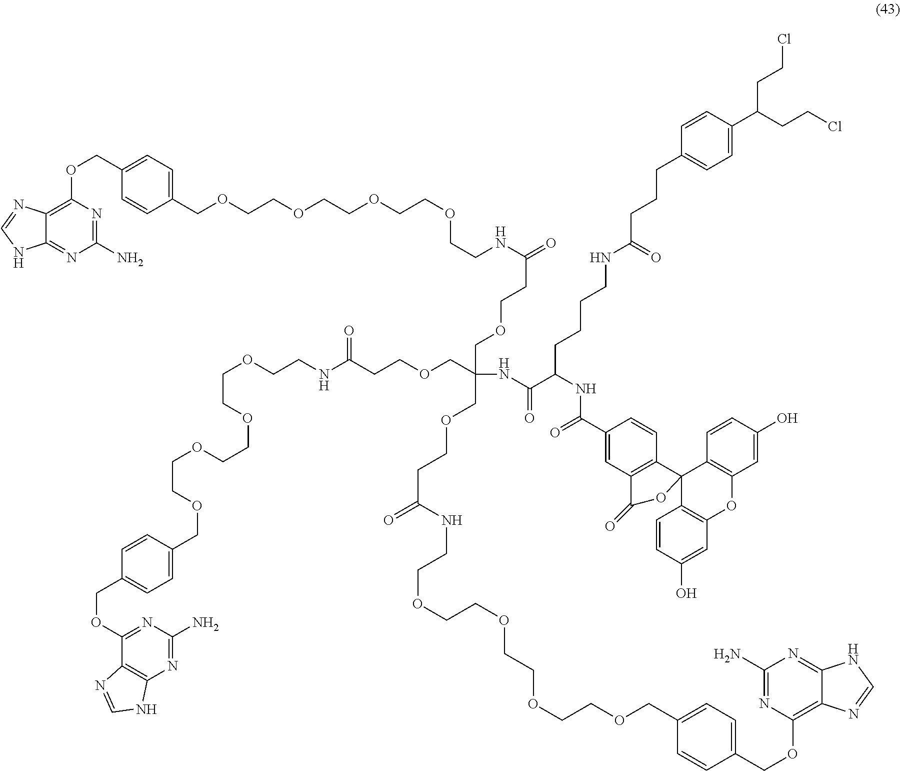

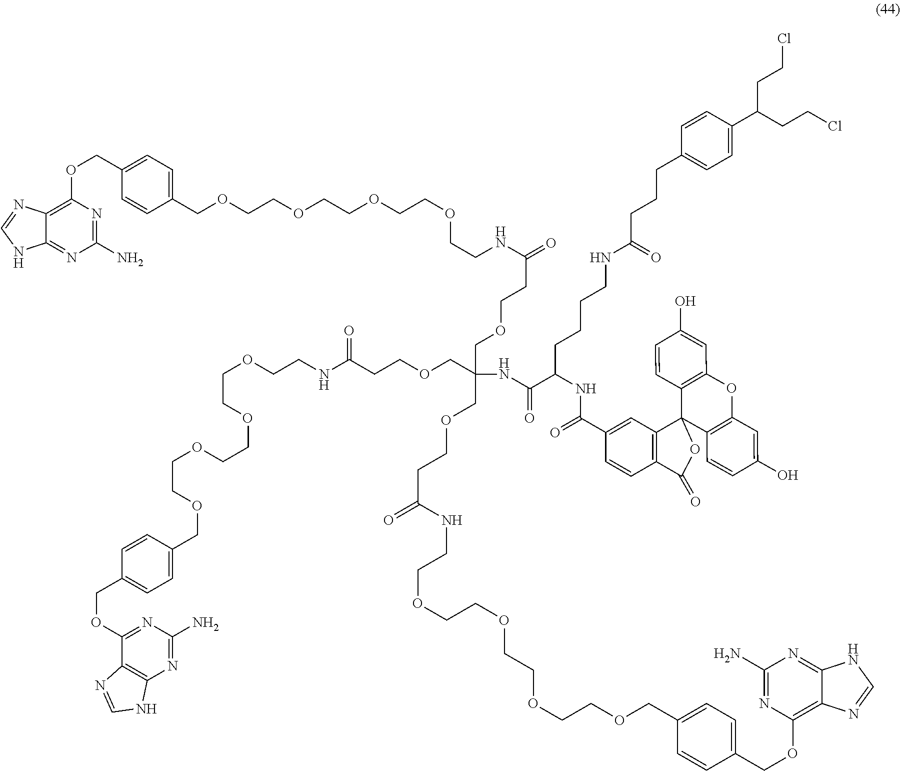

To a solution of a mixture of isomers (41) and (42) (15 mg, 0.02 mmol) in DMF (2 mL) PYBOP (10 mg, 0.02 mmol) is added at rt. The solution is stirred at rt for 20 min. Compound (5) (32 mg, 0.02 mmol) and DIPEA (3.3 μL, 0.02 mmol) are added and the solution is heated at 50° C. for 5 min. The solution is stirred at rt overnight. The solution is poured into water, the precipitate is filtered and washed with water. The desired compounds (43) and (44) are obtained as a solid. ESI-MS: m/z 2393.01 [M+H]⁺.

### Example 37

### N-5-Fluorescein-N′-6-maleimidohexanoyl-Lys-OH (45) and N-6-fluorescein-N′-6-maleimidohexanoyl-Lys-OH (46)

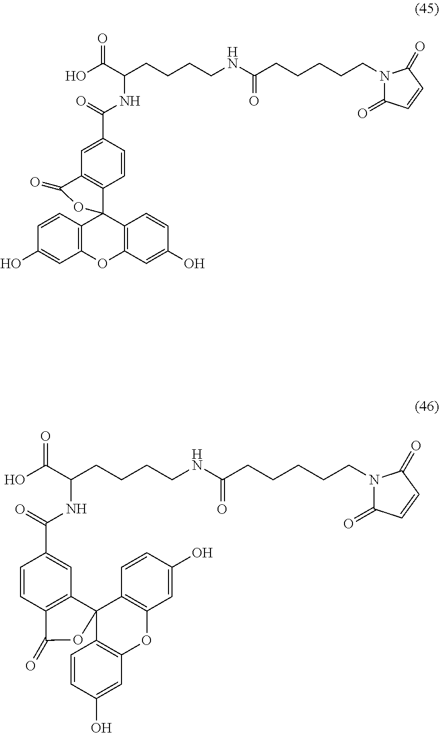

To a solution of 6-maleimido-hexanoic acid (66 mg, 0.31 mmol) in DMF (3 mL) PYBOP (161 mg, 0.31 mmol) is added at rt. The solution is stirred at rt for 20 min. The mixture of compounds (39) and (40) (156 mg, 0.31 mmol) and DIPEA (51 μL, 0.31 mmol) is added and the solution is heated at 50° C. for 5 min. The solution is stirred at rt overnight. The crude mixture is poured into water (60 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (45) and (46). ESI-MS: m/z 699.23 [M+H]⁺.

### Example 38

### N-Tris[2-(BG-PEG4-NH-carbonyl)ethyloxymethyl]methyl N′-5-fluorescein-N″-6-maleimidohexanoyl-Lys-amide (47) and corresponding 6-fluorescein derivative (48)

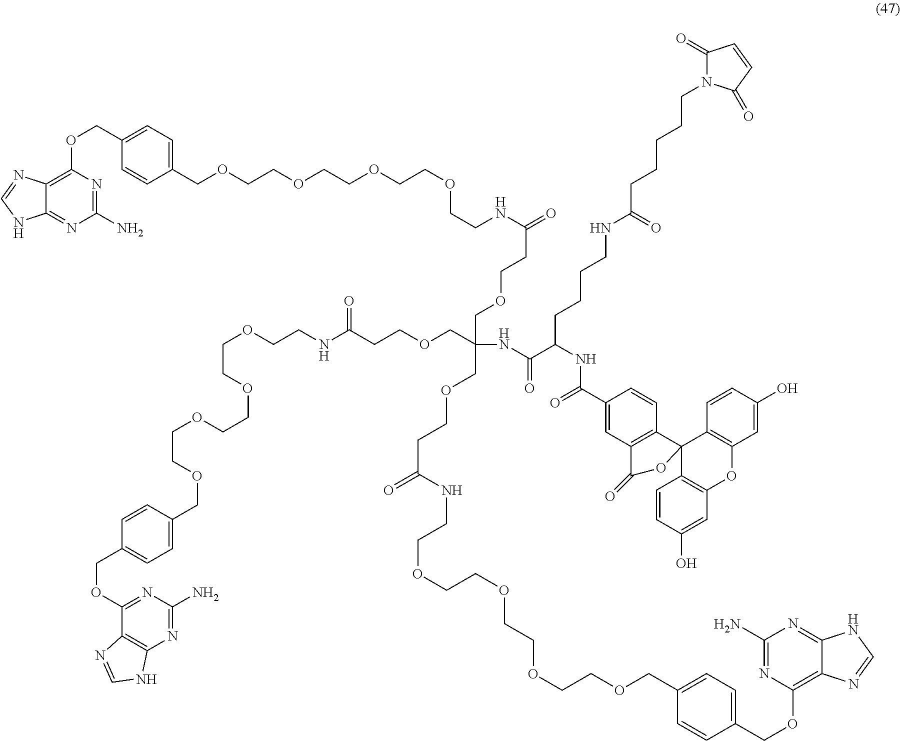

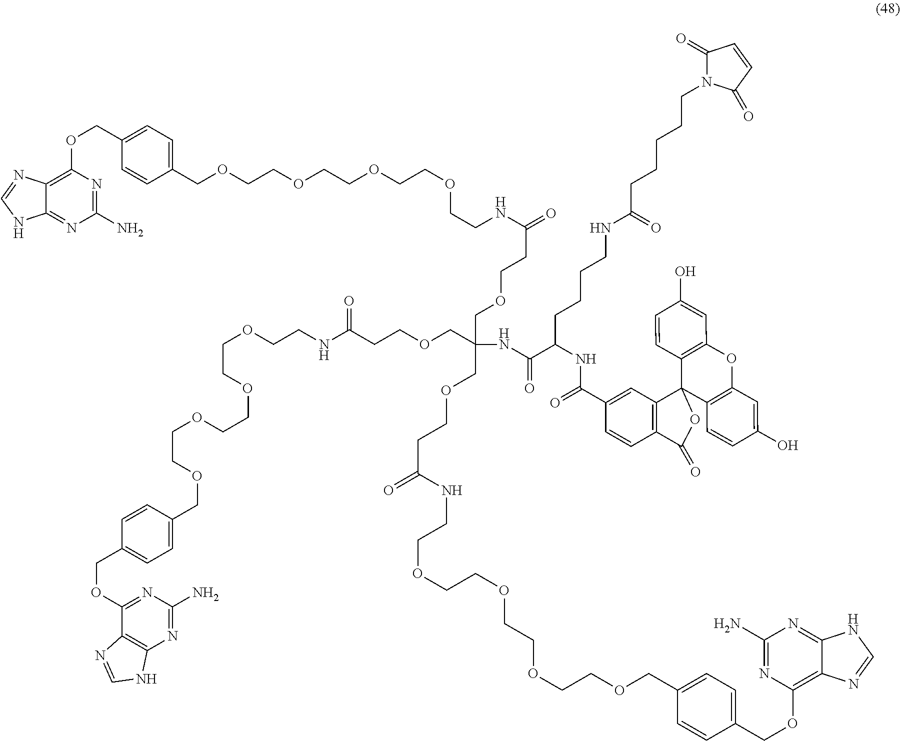

To a solution of mixture of isomers (45) and (46) (9 mg, 0.013 mmol) in DMF (2 mL) PYBOP (6.5 mg, 0.013 mmol) is added at rt. The solution is stirred at rt for 20 min. Compound (5) (21 mg, 0.013 mmol) and DIPEA (2.1 μL, 0.013 mmol) are added and the solution is heated at 50° C. for 5 min. The solution is stirred at rt overnight. The solution is poured onto water, and the precipitate is filtered and washed with water. The desired compound is obtained as a mixture of isomers (47) and (48) as a solid.

ESI-MS: m/z 2302.01 [M+H]⁺.

### Example 39

### N-Tris[2(BG-PEG4-NH-carbonyl)ethyloxymethyl]methyl N′-5-fluorescein-N″-6-(2-thiosuccinimido)hexanoyl-Lys-amide siRNA conjugate (49) and corresponding 6-fluorescein derivative (50)

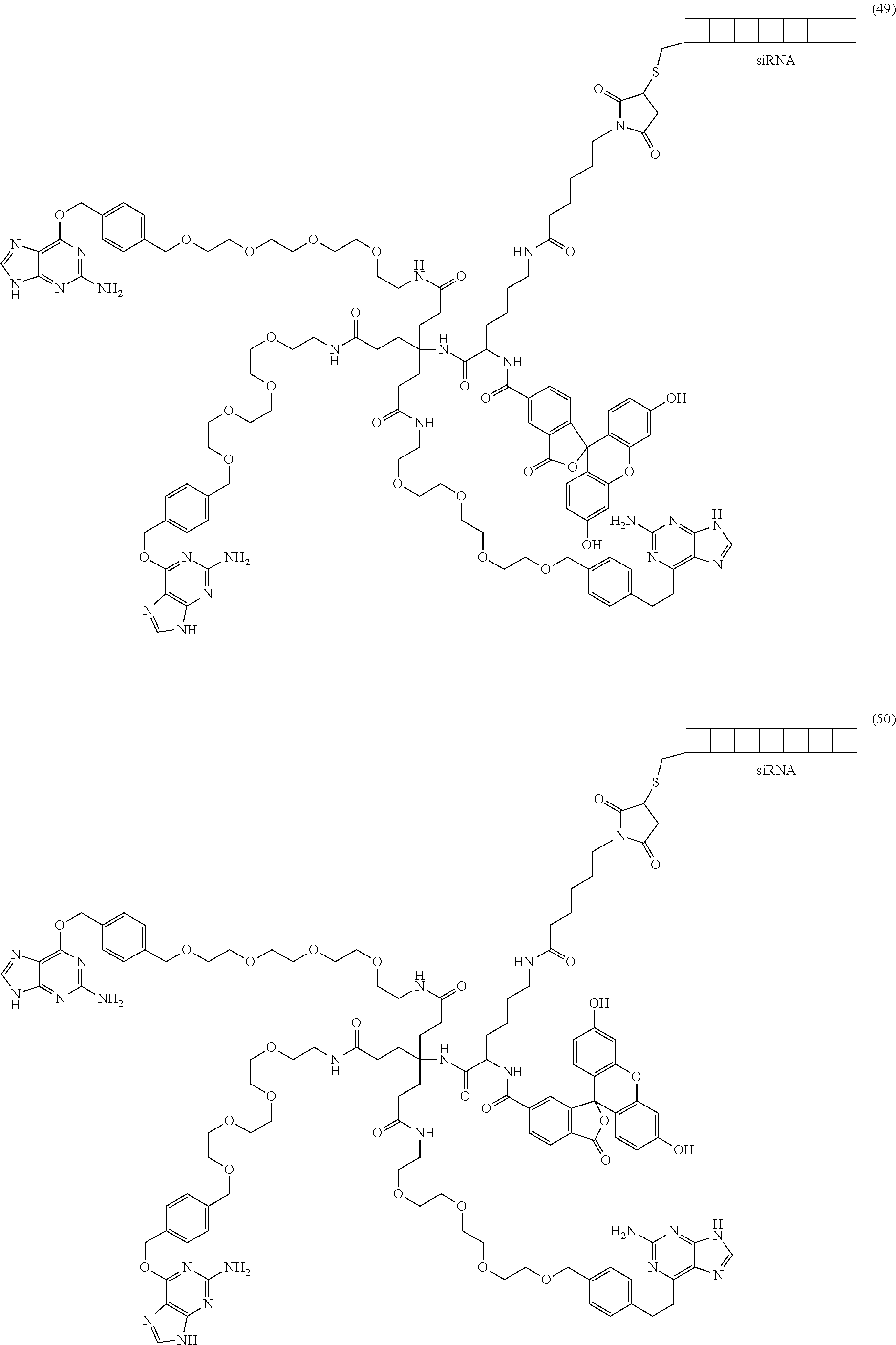

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of a mixture of isomers (47) and (48) (2.5 mM in DMF) is added and the reaction mixture incubated at room temperature for 1 h. The reaction mixture is diluted with water to a total volume of 2 mL and excess maleimide removed by gel filtration. The mixture of conjugates (49) and (50) is then purified by HPLC (solvent A: 0.1 M tetraethylammonium acetate pH 6.9 in water; solvent B: acetonitrile).

### Example 40

### N-Tris[2-(BG-PEG4-NH-carbonyl)ethyloxymethyl]methyl N′-2-{2-[2-(2-(N″-5-fluorescein-N′″-chlorambucil-Lys-amido)ethoxy)ethoxy]ethoxy}ethyl-urea (51) and corresponding 6-fluorescein derivative (52)

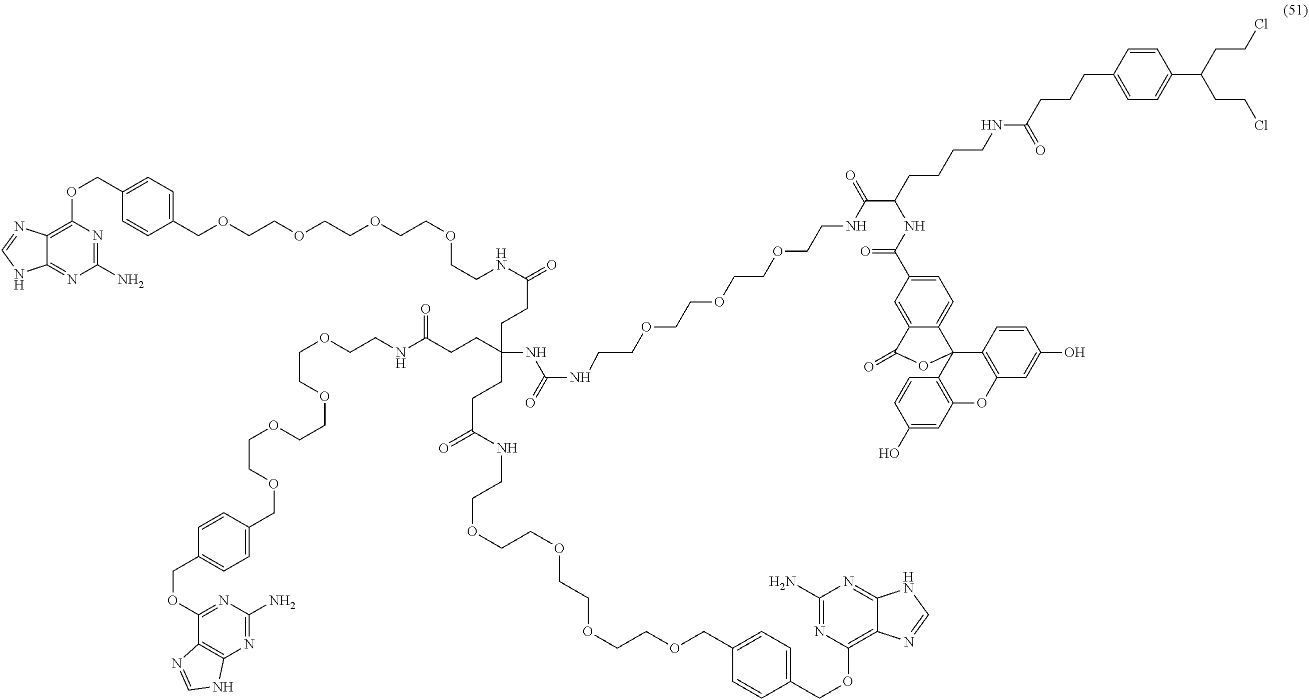

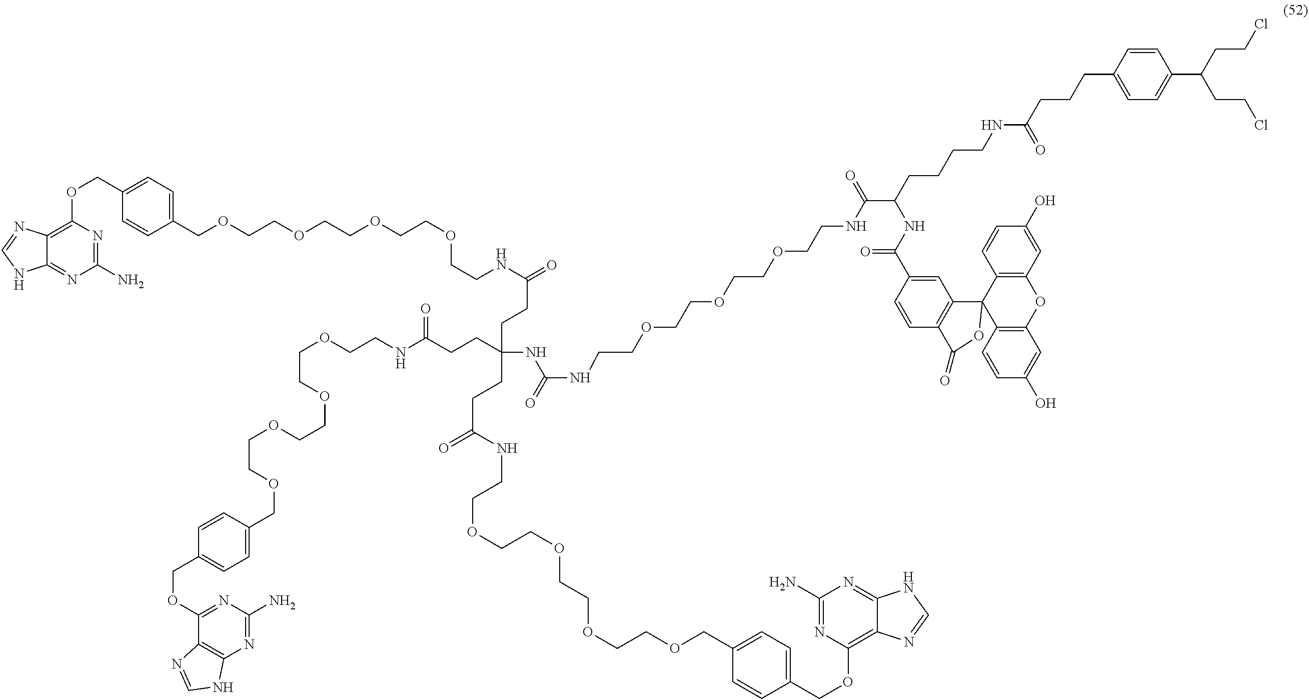

To a solution of mixture of isomers (41) and (42) (19 mg, 0.024 mmol) in DMF (3 mL) PYBOP (13 mg, 0.024 mmol) is added at rt. The solution is stirred at rt for 20 min. Compound (14) (42 mg, 0.024 mmol) and DIPEA (4 μL, 0.024 mmol) are added and the solution is heated at 50° C. for 5 min. The solution is stirred at rt overnight. The solution is poured onto water, the precipitate is filtered and washed with water. The desired compound is obtained as a mixture of isomers (51) and (52).

ESI-MS: m/z 2521.10 [M+H]⁺.

### Example 41

### N-Tris[2-(BG-PEG4-NH-carbonyl)ethyloxymethyl]methyl N′-2-{2-[2-(2-(N″-5-fluorescein-N′″-6-maleimidohexanoyl-Lys-amido)ethoxy)ethoxy]ethoxy}ethyl-urea (53) and corresponding 6-fluorescein derivative (54)

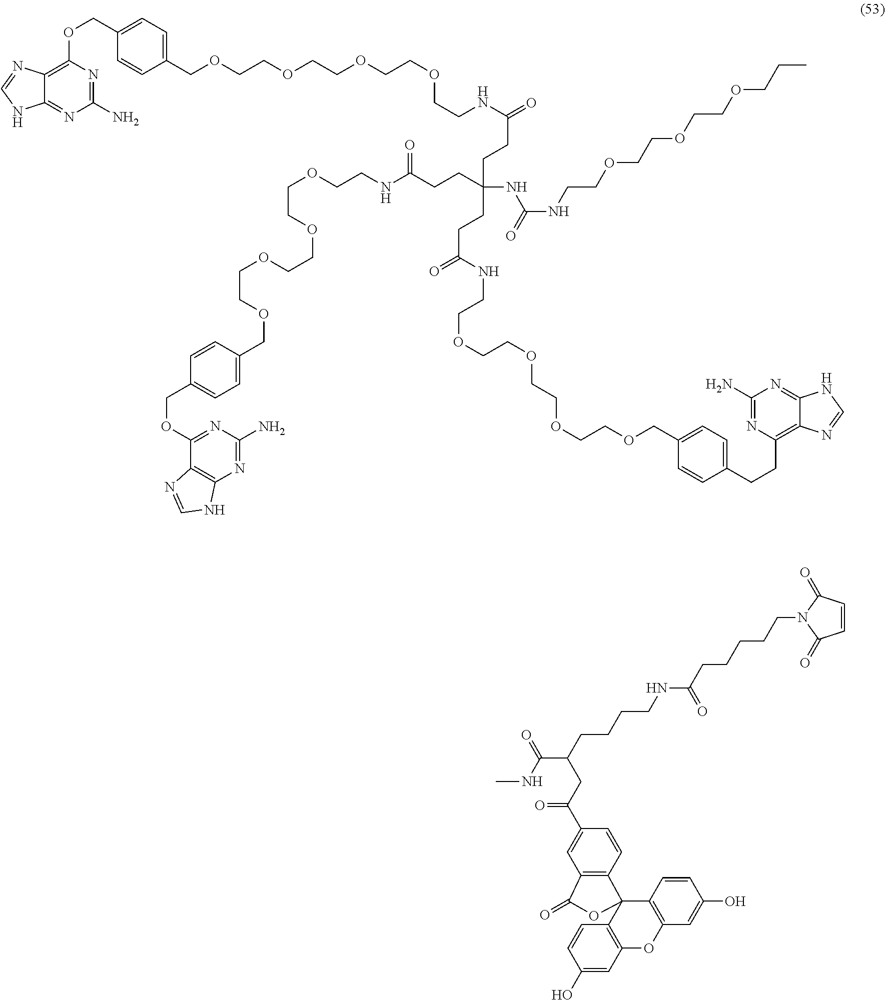

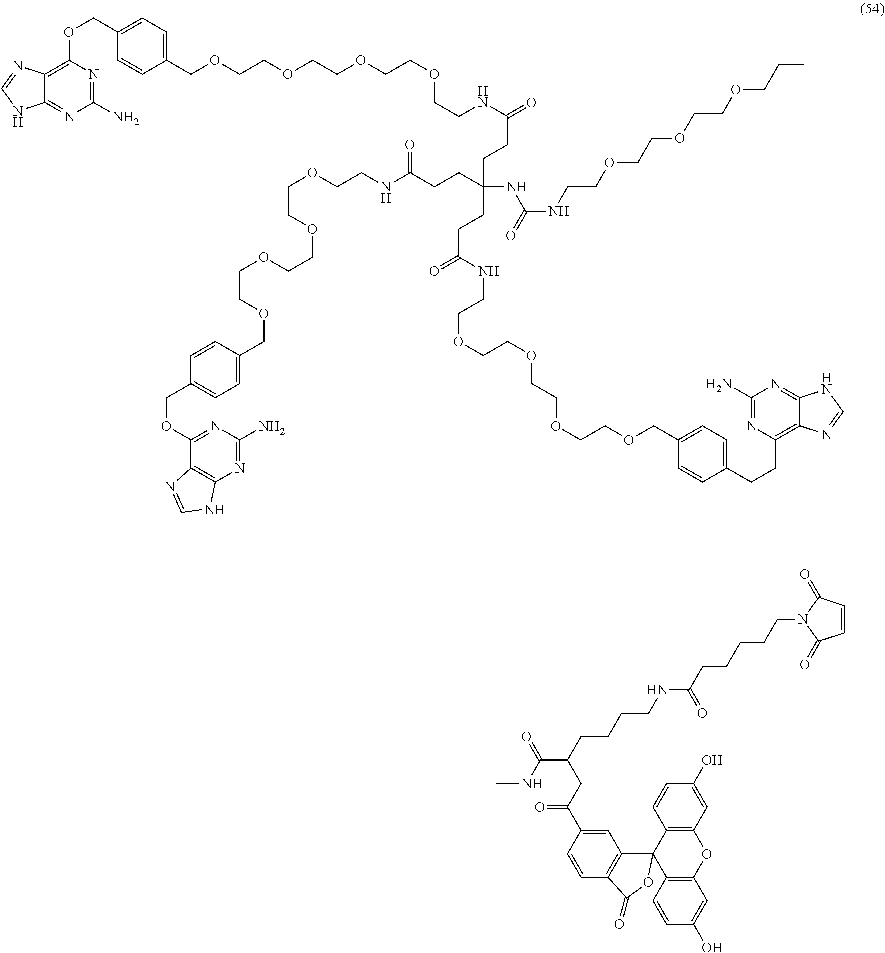

To a solution of a mixture of isomers (45) and (46) (21 mg, 0.03 mmol) in DMF (3 mL) PYBOP (16 mg, 0.03 mmol) is added at rt. The solution is stirred at rt for 20 min. Compound (14) (53 mg, 0.03 mmol) and DIPEA (5 μL, 0.03 mmol) are added and the solution is heated at 50° C. for 5 min. The solution is stirred at rt overnight. The solution is poured into water, and the precipitate is filtered and washed with water. The desired compound is obtained as a mixture of isomers (53) and (54).

ESI-MS: m/z 2430.10 [M+H]⁺.

### Example 42

### N-Tris[2-(BG-PEG4-NH-carbonyl)ethyloxymethyl]methyl N′-2-{2-[2-(2-(N″-5-fluorescein-N′″-6-(2-thiosuccinimido)hexanoyl-Lys-amido)ethoxy)ethoxy]-ethoxy}ethyl-urea siRNA conjugate (55) and corresponding 6-fluorescein derivative (56)

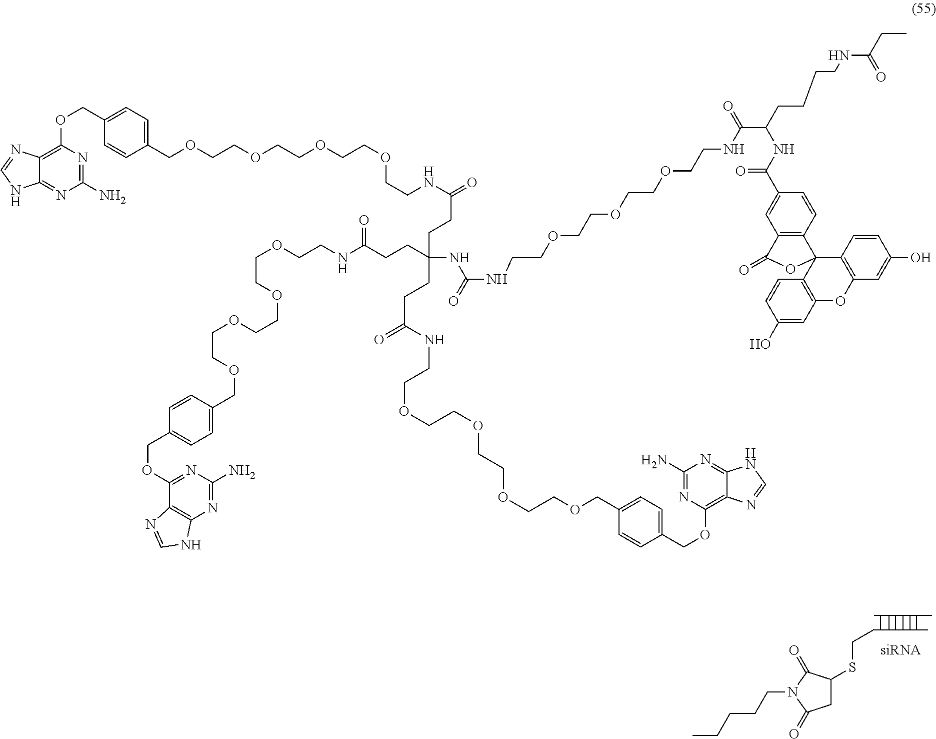

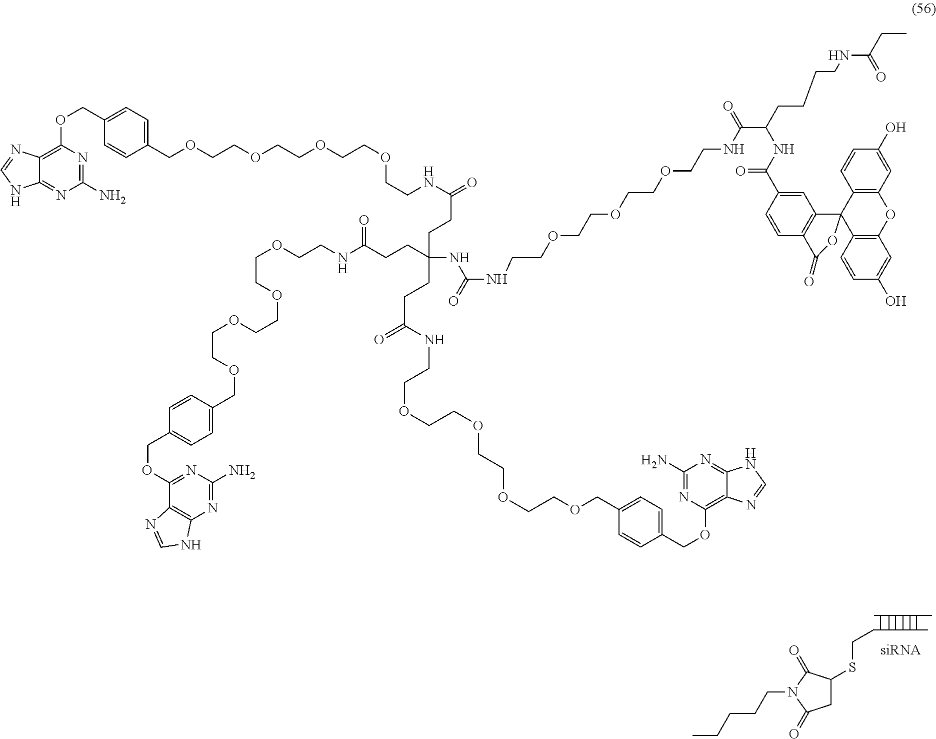

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of a mixture of isomers (53) and (54) (2.5 mM in DMF) is added and the reaction mixture incubated at room temperature for 1 h. The reaction mixture is diluted with water to a total volume of 2 mL, and excess maleimide removed by gel filtration. The mixture of conjugates (55) and (56) is then purified by HPLC (solvent A: 0.1 M tetraethylammonium acetate pH 6.9 in water; solvent B: acetonitrile).

### Example 43

### N-Tris{2-[2-(2-(CoA-S-succinimido)ethylaminocarbonyl-PEG12)ethyl-aminocarbonyl]ethoxymethyl}methyl N′-5-fluorescein-N″-chlorambucil-Lys-amide (57) and corresponding 6-fluorescein derivative (58)

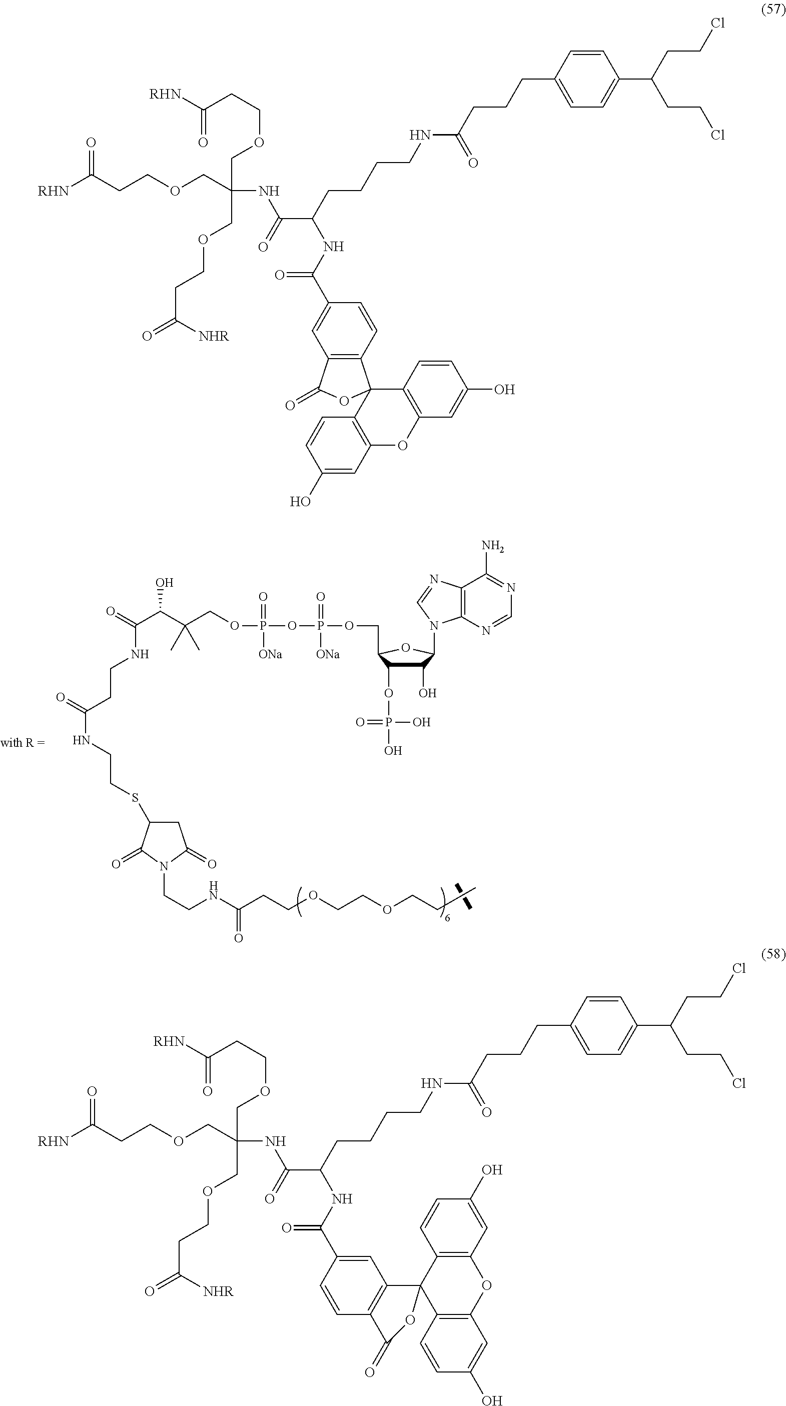

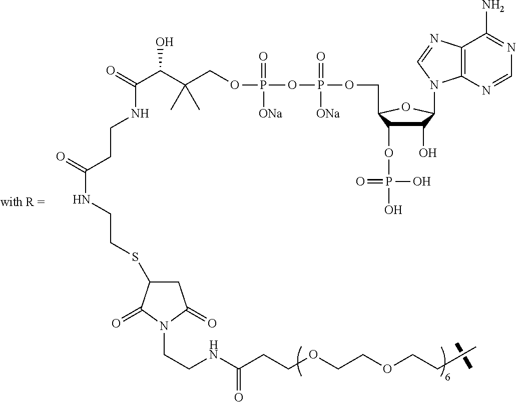

To a solution of the mixture of isomers (41) and (42) (12 mg, 0.015 mmol) in DMF (2 mL) PYBOP (8 mg, 0.015 mmol) is added at rt. The solution is stirred at rt for 20 min. Compound (24) (73 mg, 0.015 mmol) and DIPEA (2.5 μL, 0.015 mmol) are added and the solution is heated at 50° C. for 5 min. The solution is stirred at rt overnight. The solvent is removed under reduced pressure, and the compound is obtained as a mixture of isomers (57) and (58) by purification with preparative HPLC.

ESI-MS: m/z 5686.1 [M-Na]⁻.

### Example 44

### N-Tris{2-[2-(2-(CoA-S-succinimido)ethylaminocarbonyl-PEG12)ethyl-aminocarbonyl]ethoxymethyl}methyl N′-5-fluorescein-N″-6-maleimido-hexanoyl-Lys-amide (59) and corresponding 6-fluorescein derivative (60)

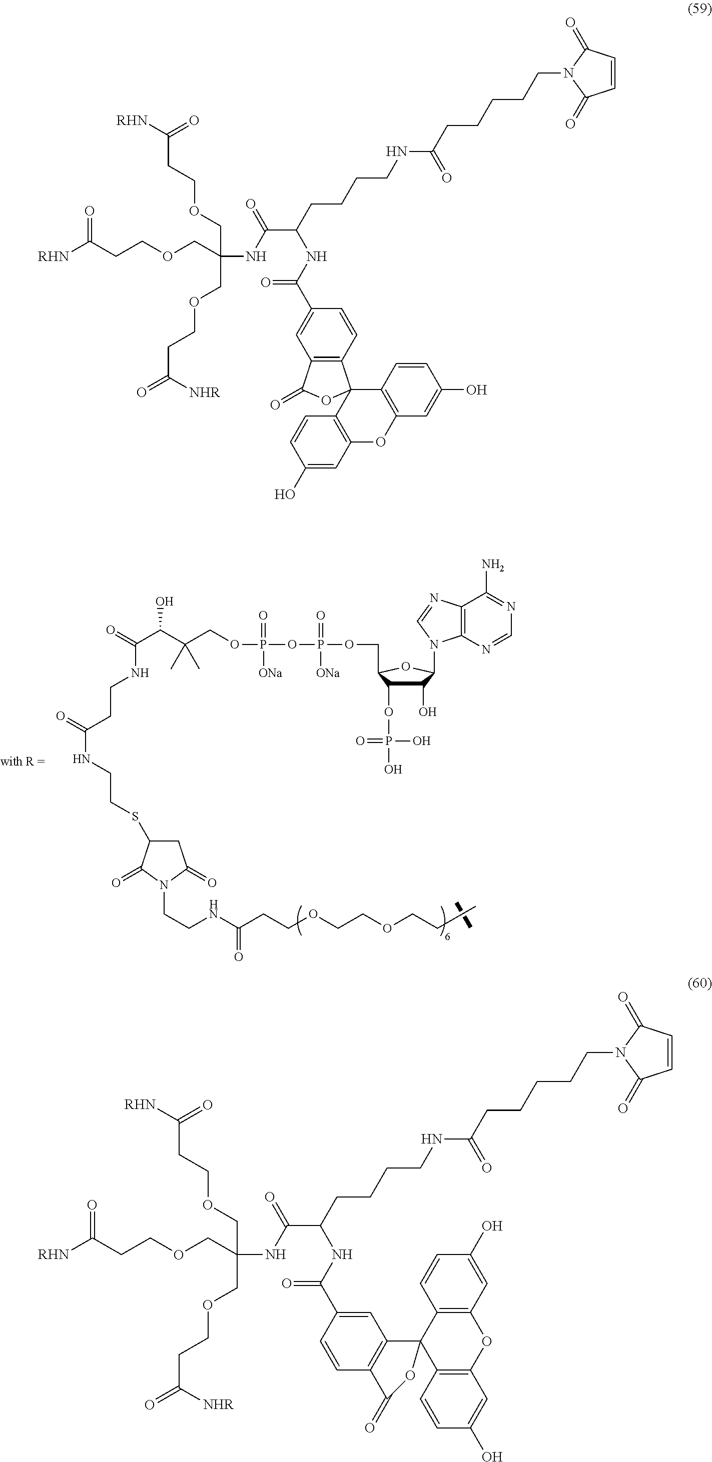

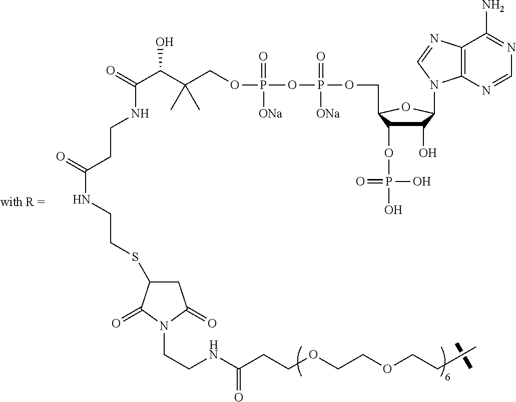

To a solution of mixture of isomers (45) and (46) (7 mg, 0.01 mmol) in DMF (2 mL) PYBOP (5 mg, 0.01 mmol) is added at rt. The solution is stirred at rt for 20 min. Compound (24) (50 mg, 0.01 mmol) and DIPEA (1.65 μL, 0.01 mmol) are added and the solution is heated at 50° C. for 5 min. The solution is stirred at rt overnight. The solvent is removed under reduced pressure, and the compound is obtained as a mixture of isomers (59) and (60) by purification with preparative HPLC.

ESI-MS: m/z 5594.1 [M-Na]⁻.

### Example 45

### N-Tris{2-[2-(2-(CoA-S-succinimido)ethylaminocarbonyl-PEG12)ethyl-aminocarbonyl]ethoxymethyl}methyl N′-5-fluorescein-N″-6-(2-thiosuccinimido)hexanoyl-Lys-amide siRNA conjugate (61) and corresponding 6-fluorescein derivative (62)

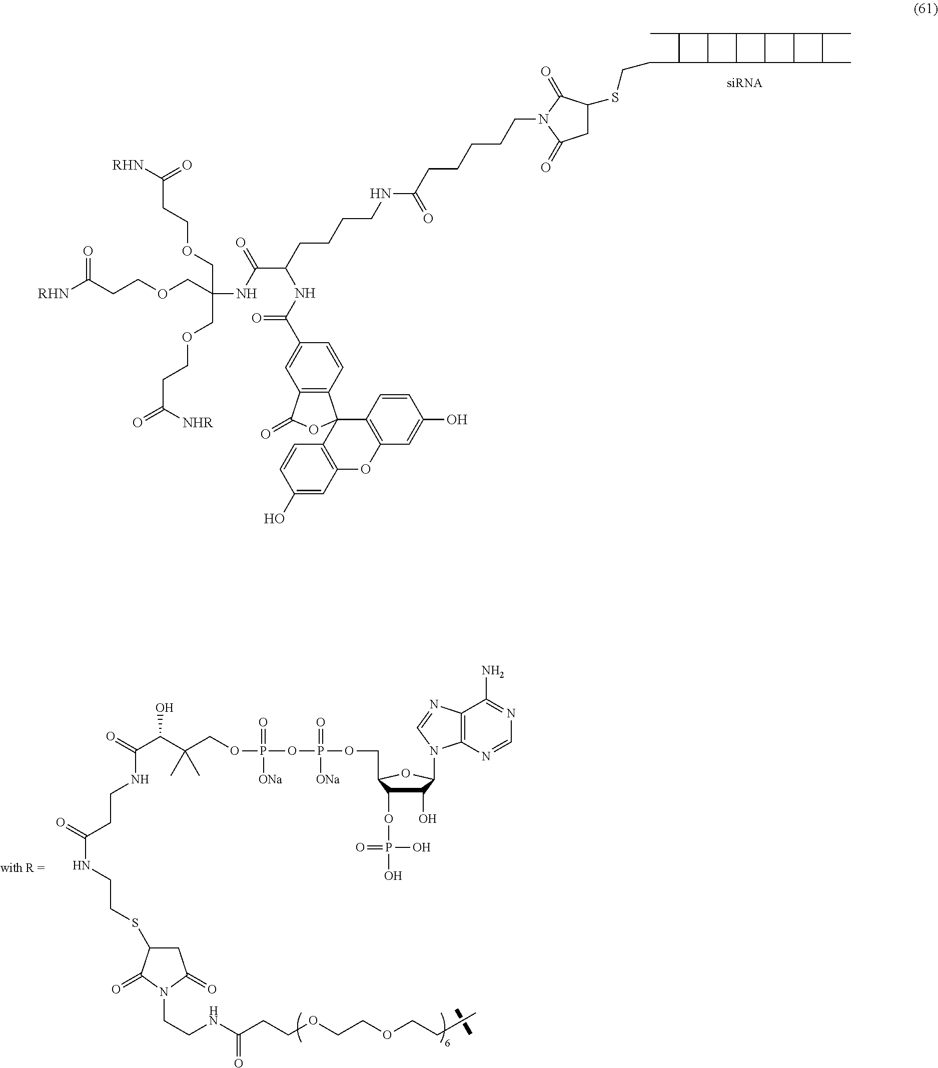

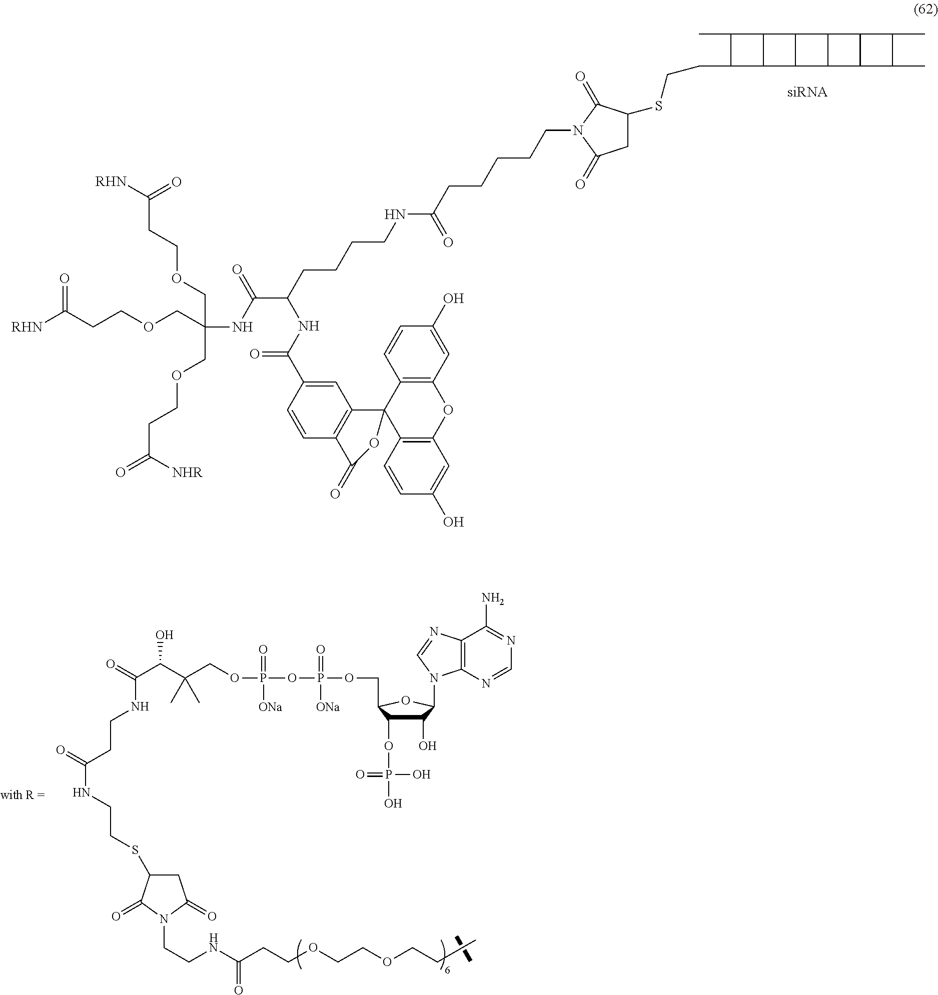

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL, 300 μL of a solution of mixture of isomers (59) and (60) (2.5 mM in DMF) is added and the reaction mixture incubated at room temperature for 1 h. The reaction mixture is diluted with water to a total volume of 2 mL, and excess maleimide removed by gel filtration. The mixture of conjugates (61) and (62) is then purified by HPLC (solvent A: 0.1 M tetraethylammonium acetate pH 6.9 in water; solvent B: acetonitrile).

### Example 46

### N-Tris{2-(2-(2-(CoA-S-succinimido)ethylaminocarbonyl-PEG12)ethyl-aminocarbonyl)ethoxymethyl}methyl N′-2-(2-(2-(2-(N″-5-fluorescein-N′″-chlorambucil-Lys-amido)ethoxy)ethoxy)ethoxy)ethyl-urea (63) and corresponding 6-fluorescein derivative (64)

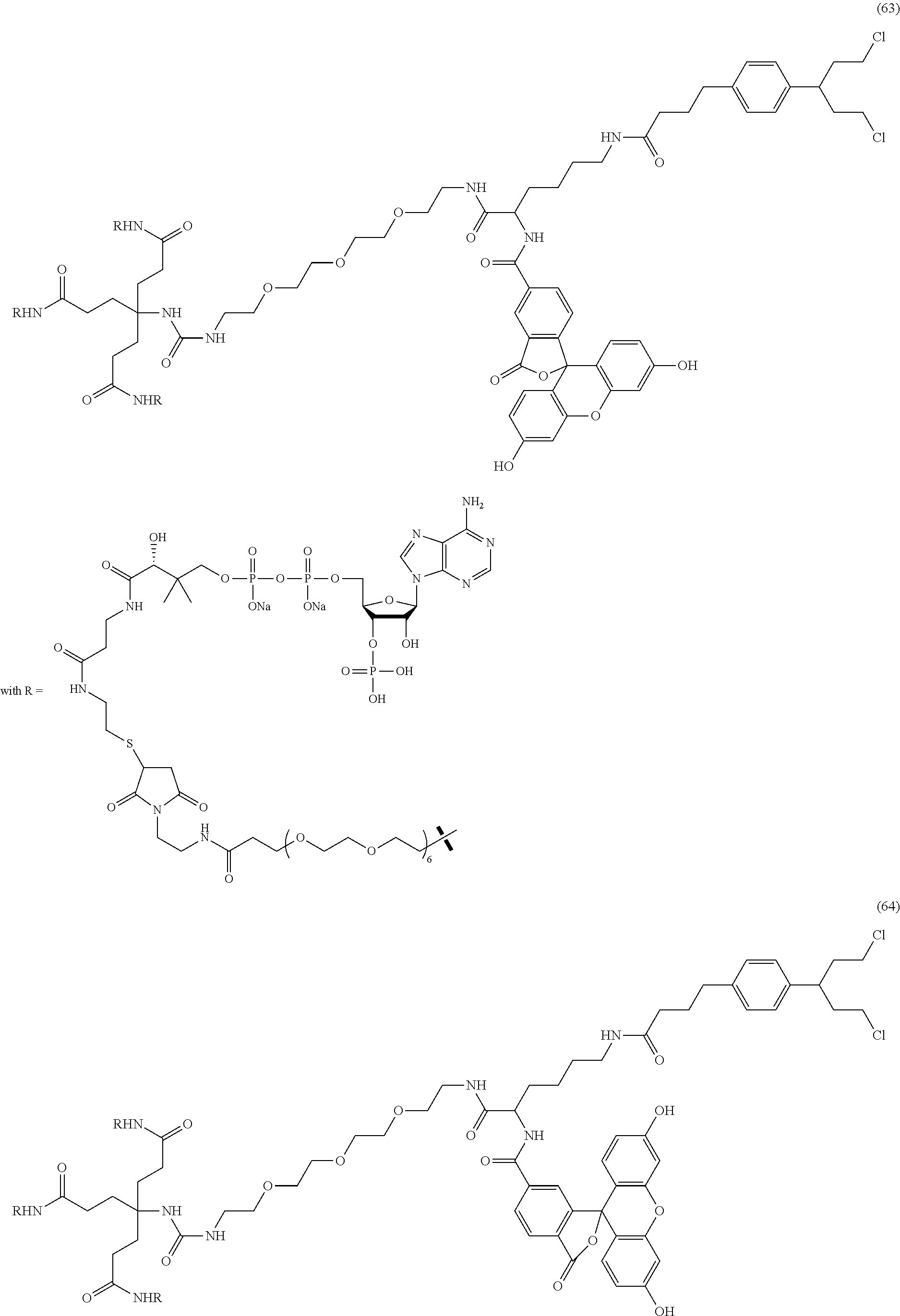

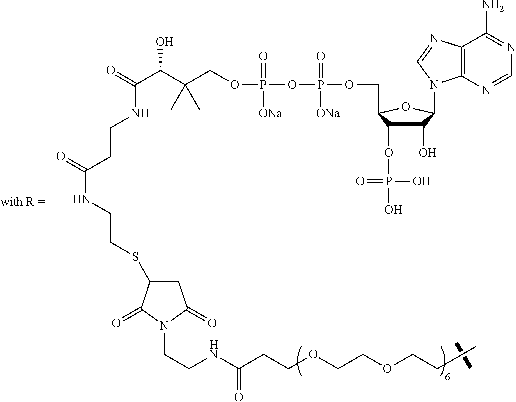

To a solution of mixture of isomers (41) and (42) (5 mg, 0.006 mmol) in DMF (1 mL) PYBOP (3 mg, 0.006 mmol) is added at rt. The solution is stirred at rt for 20 min. Compound (31) (30 mg, 0.006 mmol) and DIPEA (1 μL, 0.006 mmol) are added and the solution is heated at 50° C. for 5 min. The solution is stirred at rt overnight. The solvent is removed under reduced pressure, and the compound is obtained as a mixture of isomers (63) and (64) by purification with preparative HPLC.

ESI-MS: m/z 5814.9 [M-Na]⁻.

### Example 47

### N-Tris{2-[2-(2-(CoA-S-succinimido)ethylaminocarbonyl-PEG12)ethyl-aminocarbonyl]ethoxymethyl}methyl N′-2-{2-[2-(2-(N″-5-fluorescein-N′″-6-(2-thiosuccinimido)hexanoyl-Lys-amido)ethoxy)ethoxy]ethoxy}ethyl-urea (65) and corresponding 6-fluorescein derivative (66)

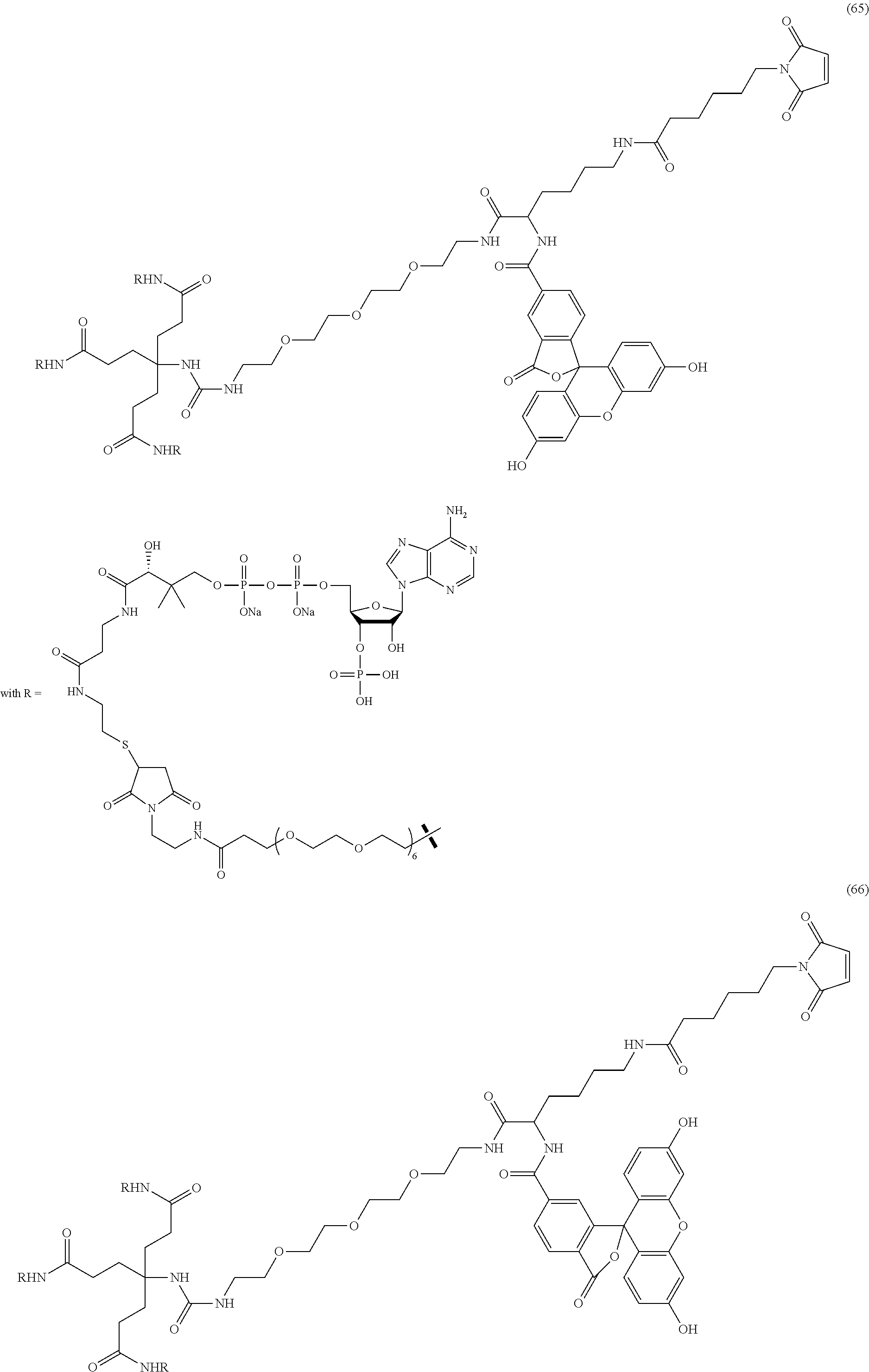

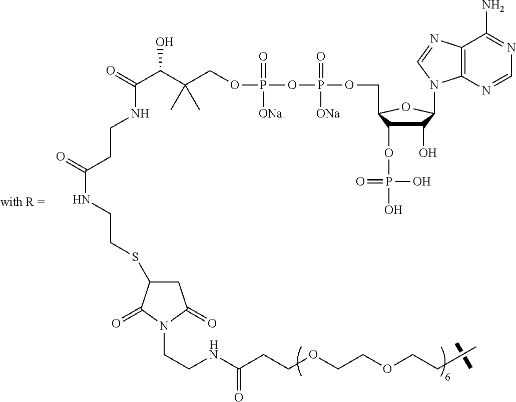

To a solution of mixture of isomers (45) and (46) (14 mg, 0.02 mmol) in DMF (2 mL) PYBOP (10 mg, 0.02 mmol) is added at rt. The solution is stirred at rt for 20 min. Compound (31) (100 mg, 0.02 mmol) and DIPEA (3.3 μL, 0.02 mmol) are added and the solution is heated at 50° C. for 5 min. The solution is stirred at rt overnight. The solvent is removed under reduced pressure, and the compound is obtained as a mixture of isomers (65) and (66) by purification with preparative HPLC.

ESI-MS: m/z 5722.2 [M-Na]⁻.

### Example 48

### N-Tris{2-[2-(2-(CoA-S-succinimido)ethylaminocarbonyl-PEG12)ethyl-aminocarbonyl]ethoxymethyl}methyl N′-2-{2-[2-(2-(N″-5-fluorescein-N′″-6-(2-thiosuccinimido)hexanoyl-Lys-amido)ethoxy)ethoxy]ethoxy}ethyl-urea siRNA conjugate (67) and corresponding 6-fluorescein derivative (68)

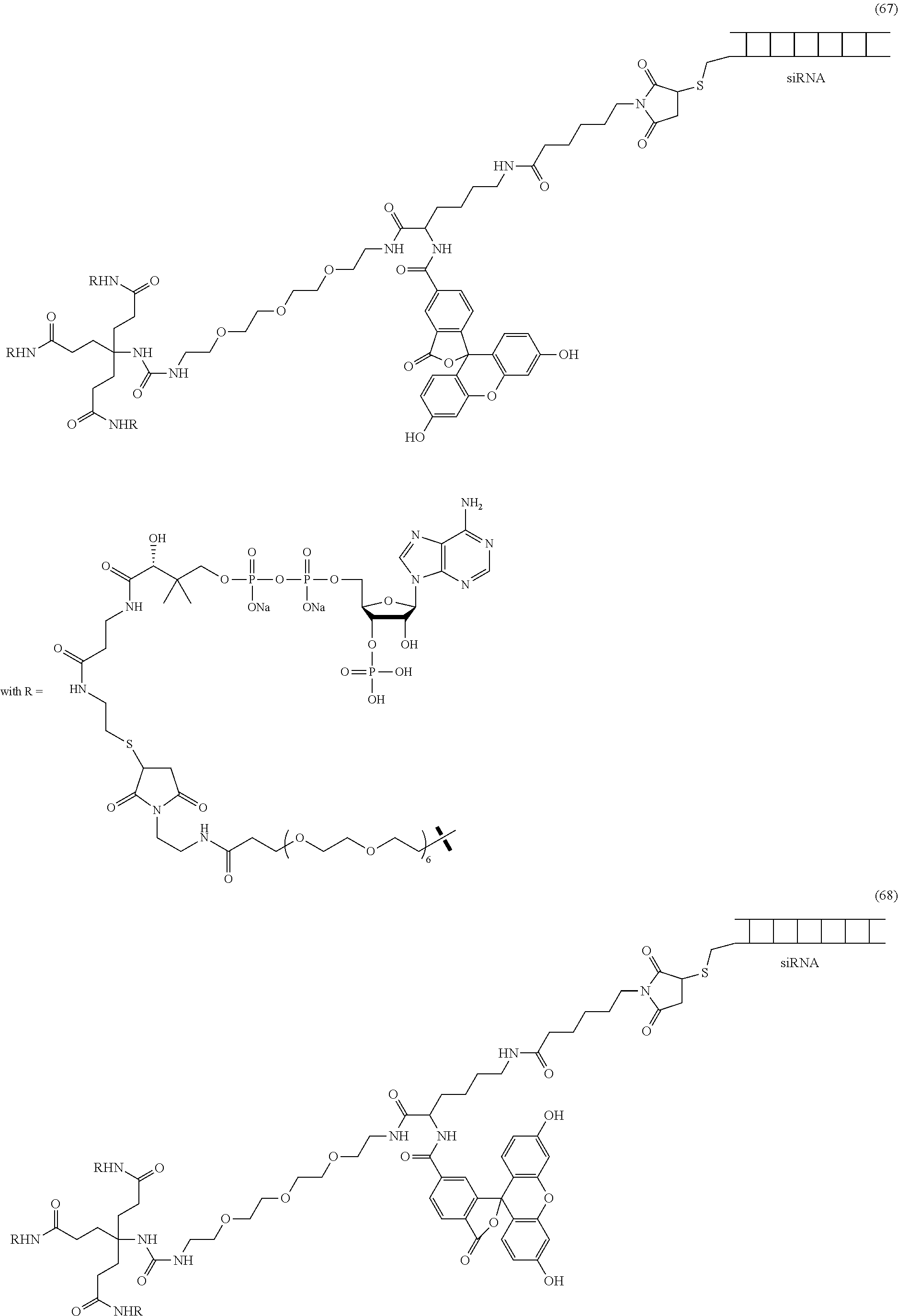

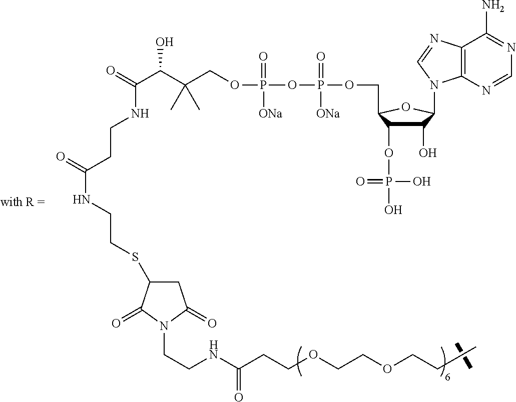

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL, 300 μL solution of a mixture of isomers (65) and (66) (2.5 mM in DMF) is added and the reaction mixture incubated at room temperature for 1 h. The reaction mixture is diluted with water to a total volume of 2 mL and excess maleimide removed by gel filtration. The mixture of conjugates (67) and (68) is then purified by HPLC (solvent A: 0.1 M tetraethylammonium acetate pH 6.9 in water; solvent B: acetonitrile).

### Example 49

### 2-Phthalimido-N—(BG-PEG4)-succinic acid monoamide (69)

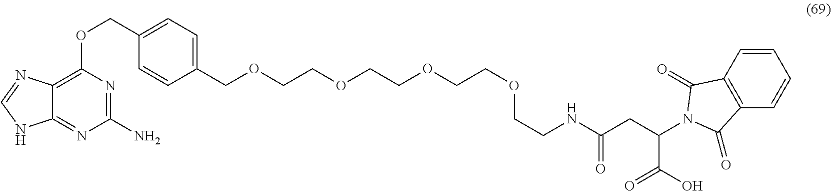

To a solution of BG-PEG4-NH2 (620 mg, 1.3 mmol, 1 eq) in DMF (15 mL) 2-phthalimido-succinic anhydride (340 mg, 1.39 mmol, 1 eq) is added at rt. The reaction mixture is stirred at rt for 4 h, and the crude mixture is poured into water (225 mL). The pH of the water phase is adjusted to pH 8 with NaOH (1 M), and the precipitate disappears. The aqueous layer is washed with ethyl acetate (2 times 100 mL). The pH is adjusted to pH 4 and the precipitate is collected. ESI-MS: m/z 692.69 [M+H]⁺.

### Example 50

### N-4-((4-Aminopyrimidin-2-yloxy)methyl)benzyl-N′-2-{2-[2-(2-azidoethoxy)-ethoxy]ethoxy}ethyl-urea (70)

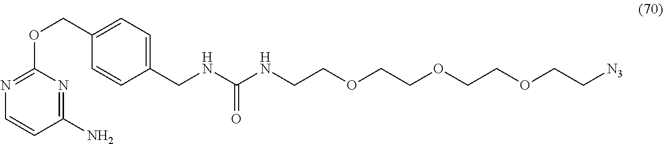

To a solution of 11-azido-3,6,9-trioxaundecan-1-amine (73 μL, 1 eq, 0.37 mmol) in DMF (3 mL) CDI (60 mg, 1 eq, 0.37 mmol) is added. The solution is stirred overnight at rt. BC—NH2 (85 mg, 1 eq, 0.37 mmol) is added to the solution, and the mixture is heated at 65° C. for 3 h. The crude mixture is poured into water and extracted with ethyl acetate, the organic phase is dried over MgSO₄, and evaporated under reduced pressure to afford the desired compound. No further purification is required.

TLC (CH₂Cl₂/MeOH 10:1). ESI-MS: m/z 475.51 [m+H]⁺.

### Example 51

### N-4-((4-Aminopyrimidin-2-yloxy)methyl)benzyl-N′-2-{2-[2-(2-aminoethoxy)-ethoxy]ethoxy}ethyl-urea (71)

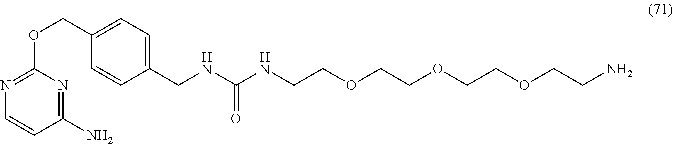

To a solution of compound (70) (54 mg, 0.12 mmol) in dioxane (3 mL) is added water (360 μL). Then PMe₃ (720 μL 1 M in THF solution, 6 eq) is added and the solution is stirred at rt for 2 h. The solvent is removed under reduced pressure, and the compound (71) is obtained by purification with preparative HPLC. ESI-MS: m/z 449.52 [M+H]⁺.

### Example 52

### N-4-((4-Aminopyrimidin-2-yloxy)methyl)benzyl-N′-2-{2-[2-(2-(3-BG-PEG4-NH-carbonyl-2-phthalimido-propionylaminoethoxy)ethoxy]ethoxy}ethyl-urea (72)

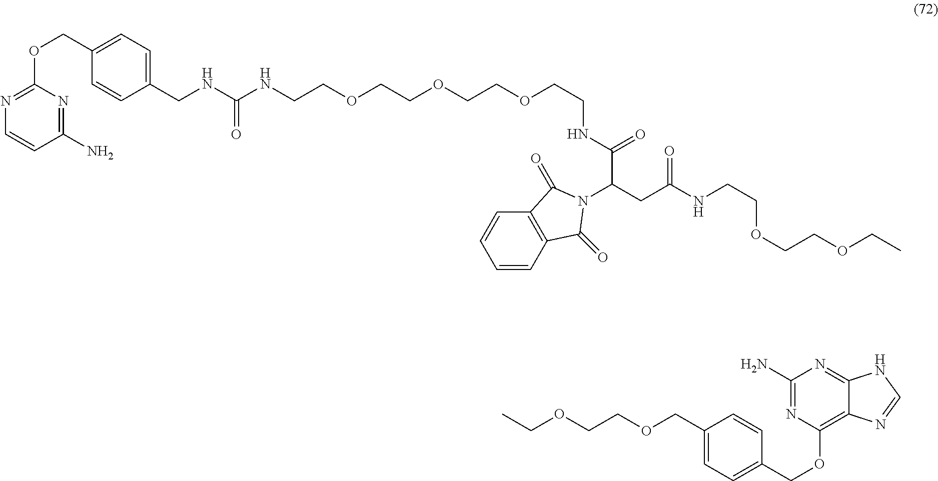

To a solution of compound (71) (45 mg, 0.1 mmol, 1 eq) and compound (69) (69 mg, 0.1 mmol, 1 eq) in DMF (2 mL) are successively added DIPEA (17 μL, 0.1 mmol, 1 eq), HOBT (1 M in NMP, 0.1 mL, 0.1 mmol, 1 eq) and DCC (21 mg, 1 mmol, 1 eq) at rt. The resulting mixture is stirred overnight. The solvent is removed under reduced pressure and the mixture is diluted with 50 mL ethyl acetate. The organic layer is washed with water, dried over MgSO₄ and evaporated under reduced pressure. Flash chromatography (CH₂Cl₂/methanol, 10/1→5/1) gives the desired compound (72).

ESI-MS: m/z 1123.19 [M+H]⁺.

### Example 53

### N-4-((4-Aminopyrimidin-2-yloxy)methyl)benzyl-N′-2-{2-[2-(2-(3-BG-PEG4-NH-carbonyl-2-amino-propionylaminoethoxy)ethoxy]ethoxy}ethyl-urea (73)

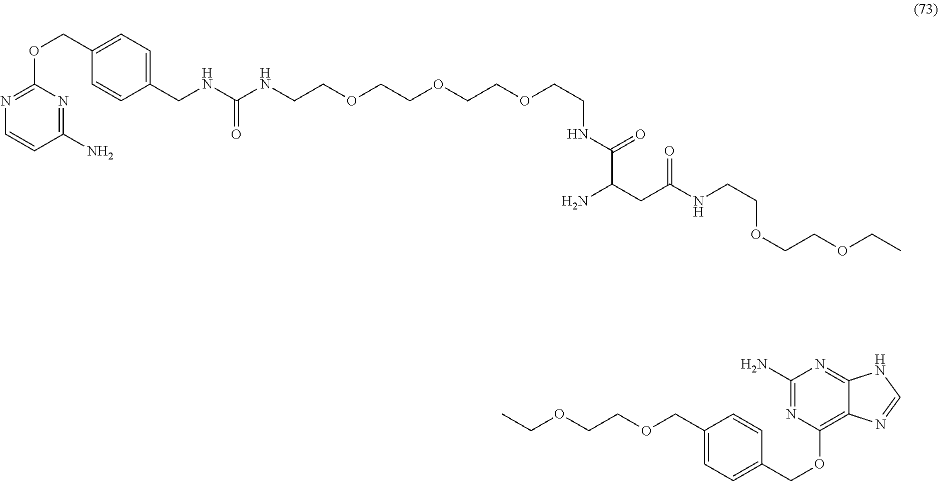

To a solution of compound (72) (45 mg, 0.04 mmol) in ethanol (3 mL) methylamine (300 μl) is added, and the solution is stirred at rt for 12 h. The solvent is removed under reduced pressure and the compound (73) is obtained by purification with preparative HPLC. ESI-MS: m/z 993.09 [M+H]⁺.

### Example 54

### N-4-((4-Aminopyrimidin-2-yloxy)methyl)benzyl-N′-2-{2-[2-(2-(3-BG-PEG4-NH-carbonyl-2-(fluorescein-5-carboxamido)propionylaminoethoxy)-ethoxy]ethoxy}ethyl-urea (74) and corresponding fluorescein-6-carboxamide (75)

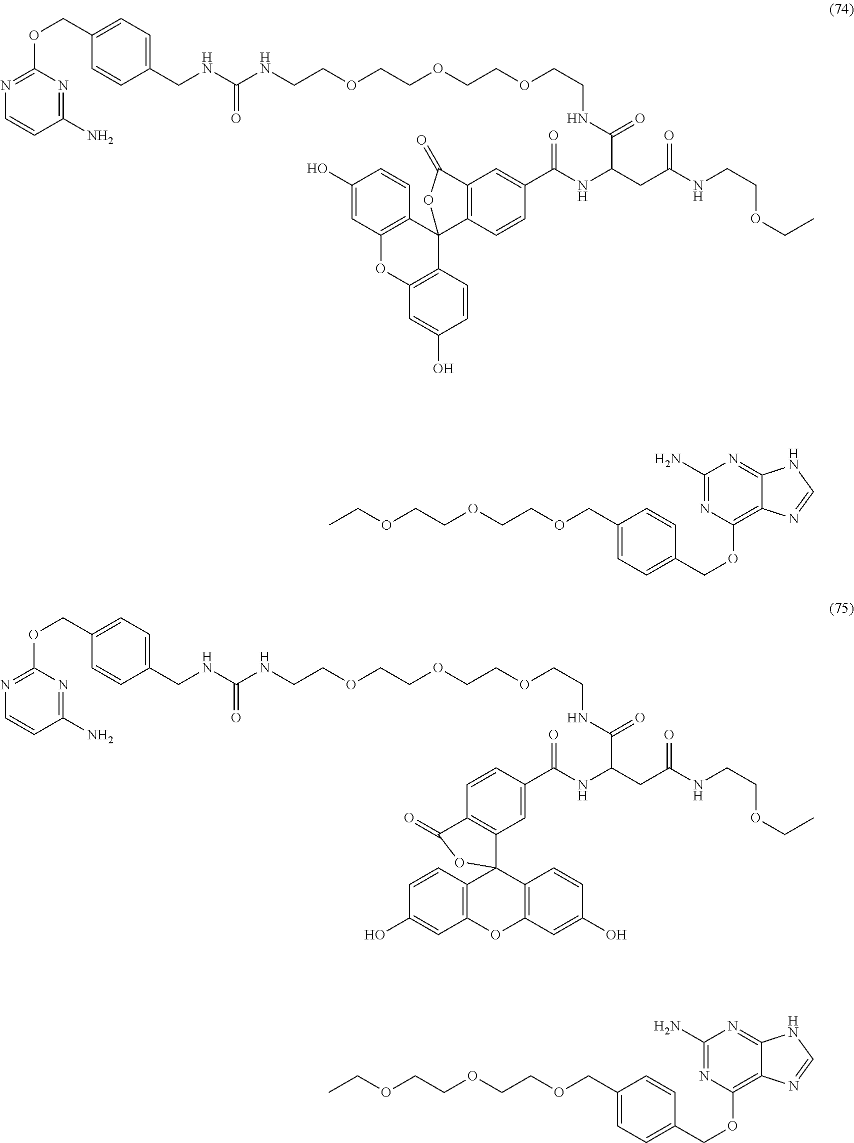

Compound (73) (9 mg, 0.009 mmol) and 5(6)-carboxyfluorescein NHS ester (4 mg, 0.009 mmol) are dissolved in 800 μL DMF with Et₃N (1.35 μL, 0.009 mmol) and heated overnight at 31° C. The solvent is evaporated under vacuum and compounds (74) and (75) isolated by reversed phase HPLC on a C18 column using a linear gradient of water:acetonitrile (from 95:5 to 20:80 in 20 min, 0.08% TFA).

ESI-MS: m/z 1351.39 [M+H]⁺.

### Example 55

### N-4-((4-Aminopyrimidin-2-yloxy)methyl)benzyl-N′-2-{2-[2-(2-(3-BG-PEG4-NH-carbonyl-2-(6-maleimidohexanoylamino)propionylaminoethoxy)ethoxyl]-ethoxy}ethyl-urea (72)

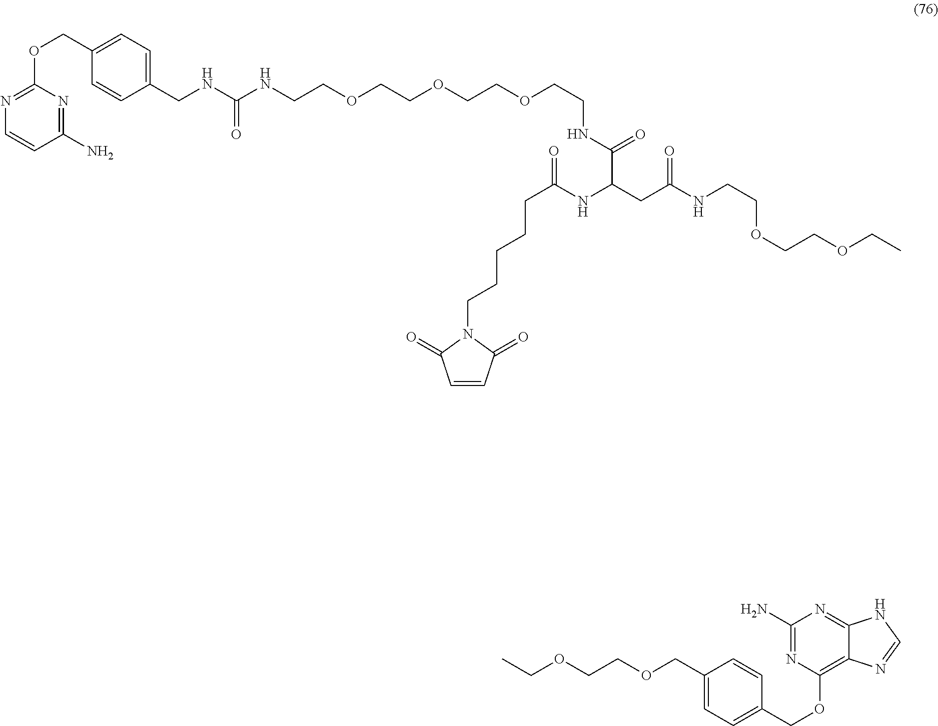

To a solution of 6-maleimido-hexanoic acid (4.4 mg, 0.02 mmol) in DMF (1 mL) PYBOP (10 mg, 0.02 mmol) is added at rt. The solution is stirred at rt for 20 min. Compound (73) (20 mg, 0.02 mmol) and DIPEA (3.3 μL, 0.02 mmol) are added and the solution is heated at 50° C. for 5 min. The solution is stirred at rt overnight. The solvent is removed under reduced pressure and the mixture is diluted with 150 mL ethyl acetate. The organic layer is washed with water, dried over MgSO₄ and evaporated under reduced pressure. Flash chromatography (CH₂Cl₂/methanol, 10/1→5/1) gives the desired compound (76). ESI-MS: m/z 1186.29 [M+H]⁺.

### Example 56

### N-4-((4-Aminopyrimidin-2-yloxy)methyl)benzyl-N′-2-{2-[2-(2-(3-BG-PEG4-NH-carbonyl-2-(6-maleimidohexanoylamino)propionylaminoethoxy)ethoxyl]-ethoxy}ethyl-urea siRNA conjugate (77)

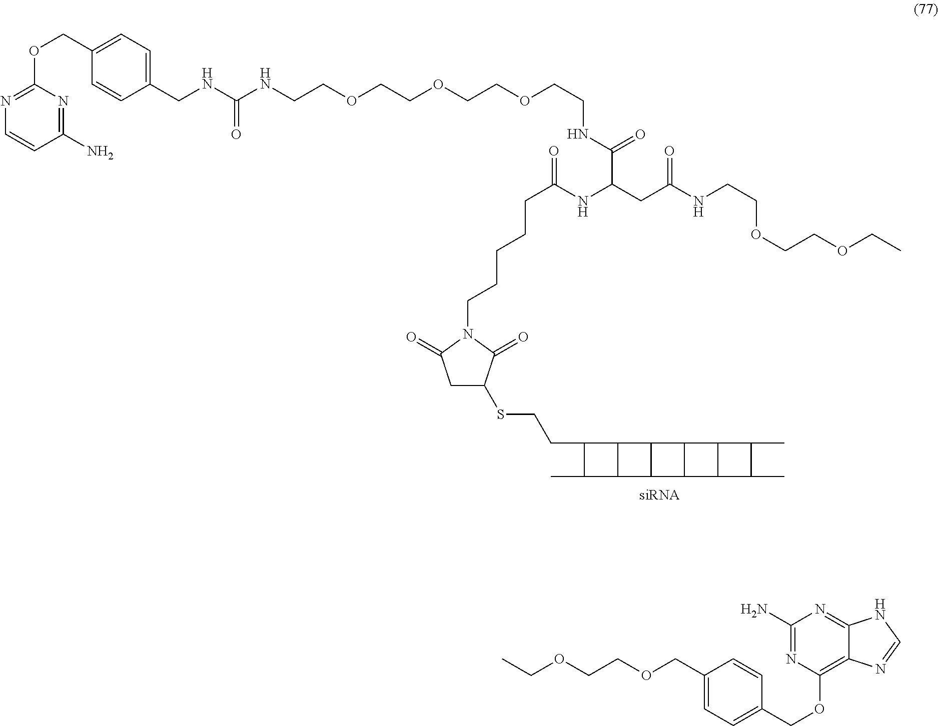

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of compound (76) (2.5 mM in DMF) is added and the reaction mixture incubated at room temperature for 1 h. The reaction mixture is diluted with water to a total volume of 2 mL and excess maleimide removed by gel filtration. The conjugate (77) is then purified by HPLC (solvent A: 0.1 M tetraethyl-ammonium acetate pH 6.9 in water; solvent B: acetonitrile).

### Example 57

### N-4-((4-Aminopyrimidin-2-yloxy)methyl)benzyl-N′-2-(2-(2-(2-(3-BG-PEG4-NH-carbonyl-2-chlorambucilcarboxamino-propionylaminoethoxy)ethoxy)-ethoxy)ethyl-urea (78)

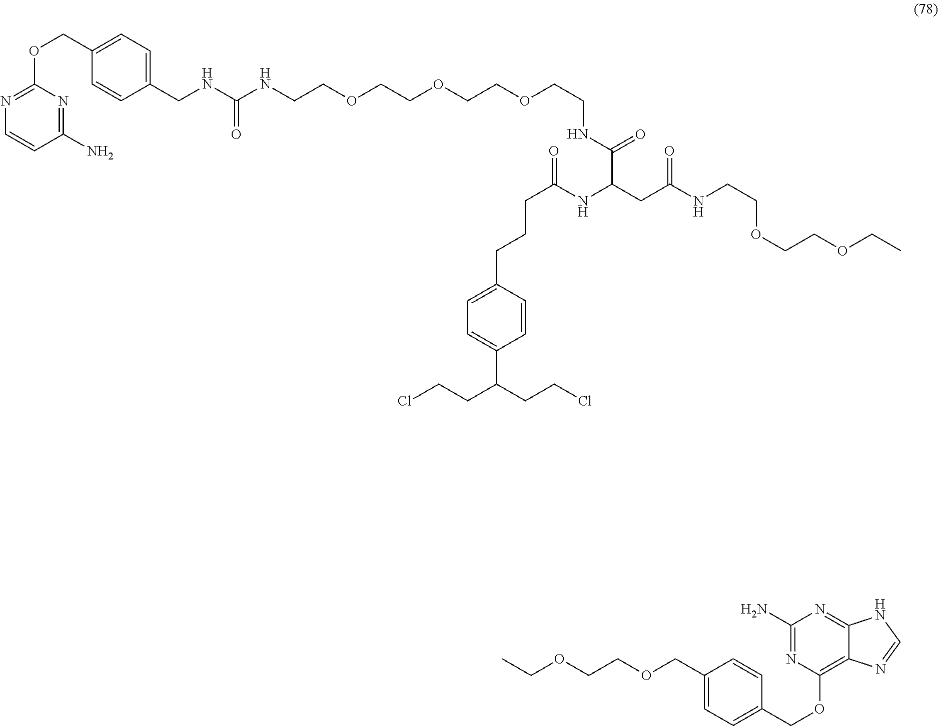

To a solution of chlorambucil (6 mg, 0.02 mmol) in DMF (1 mL) PYBOP (10 mg, 0.02 mmol) is added at rt. The solution is stirred at rt for 20 min. Compound (73) (20 mg, 0.02 mmol) and DIPEA (3.3 μL, 0.02 mmol) are added and the solution is heated at 50° C. for 5 min. The solution is stirred at rt overnight. The solvent is removed under reduced pressure and the mixture is diluted with 150 mL of ethyl acetate. The organic layer is washed with water, dried over MgSO₄ and evaporated under reduced pressure. Flash chromatography (CH₂Cl₂/methanol, 10/1→5/1) gives the desired compound (78).

ESI-MS: m/z 1276.56 [M+H]⁺.

### Example 58

### Molecular Shuttle Comprising Three Fusion Proteins of AGT with FKBP and Fluorescein as Cargo

The purified mixture of compounds (6) and (7) of Example 6 comprising three identical benzylguanine groups representing an enzyme substrate for a derivative of AGT and further comprising fluorescein is mixed in PBS, pH 7.4, containing 1 mM DTT, at a concentration of 10 μM with 50 μM of a fusion protein comprising FKBP as the proteinaceous binding entity (DNA coding for FKBP obtained from Ariad Pharmaceuticals, USA) fused to a variant of AGT available from Covalys as SNAP26 representing the enzyme subunit of the fusion protein. The mixture is reacted for 24 h in the dark. The mixture is separated using gel permeation chromatography. The separation system is set up by reacting a small quantity of about 10 μM of the compounds of Example 6 with about 15 μM of fusion protein and separating this. The retention time for the completely modified cargo structure is recorded and used for subsequent purification of the reaction mixture. The peak corresponding to a completely modified shuttle structure is isolated and stored for further use at 4° C. in the dark.

### Example 59

### Molecular Shuttle Comprising Three Fusion Proteins of AGT with FKBP and Chlorambucil and Fluorescein as Cargo

The purified mixture of compounds (43) and (44) of Example 36 comprising three identical benzylguanine groups representing an enzyme substrate for a derivative of AGT and further comprising both chlorambucil and fluorescein is mixed in PBS, pH 7.4, containing 1 mM DTT, at a concentration of 10 μM with 50 μM of the fusion protein comprising FKBP and a variant of AGT (SNAP26) as in Example 58. The mixture is reacted for 24 h in the dark. The mixture is separated using gel permeation chromatography. The peak corresponding to a completely modified shuttle structure is isolated and stored for further use at 4° C. in the dark.

### Example 60

### Molecular Shuttle Comprising One Fusion Protein of AGT with FKBP, One Fusion Protein of ACT with MEK 1 and Fluorescein as Cargo

The purified mixture of compounds (74) and (75) of Example 54 comprising one benzylguanine group representing an enzyme substrate for a derivative of AGT and one benzylcytosine group representing an enzyme substrate for an ACT further comprising fluorescein is mixed in PBS, pH 7.4, containing 1 mM DTT, at a concentration of 10 μM with 15-20 μM of the fusion protein comprising FKBP and a variant of AGT (SNAP26) and 15-20 μM of the fusion protein MEK1/Alkyl Cytosine Transferase (ACT) described in PCT/EP2007/057597. The mixture is reacted for 24 h in the dark. The mixture is separated using gel permeation chromatography. The peak corresponding to a completely modified shuttle structure is isolated and stored for further use at 4° C. in the dark.

### Example 61

### Binding Properties of a Molecular Shuttle Comprising Three Fusion Proteins of AGT with FKBP and Fluorescein as Cargo

A microplate is modified by absorbing recombinantly expressed FRB with a polyhistidine tag. The plasmids for rapamycin dependent interaction of FRB are available from Ariad Pharmaceuticals, USA. A range of densities is established according to standard ELISA protocols. After washing and blocking according to standard ELISA protocols, the shuttle comprising three FKBP units of Example 58 is incubated on this surface. For comparison a structure containing just one binder molecule and a fluorescent compound is incubated in another well. Other comparative assays are done with 50 nM of rapamycin and without rapamycin. While the monomeric unit binds only in the presence of rapamycin which increases the interaction to high affinity (KD about 10 nM), only the unit which is trimeric for FRB binds without rapamycin (KD about 10 μM). Thus the avidity of the shuttle structure is significantly increased with respect to the single binder protein.

### Example 62

### Binding Properties of a Molecular Shuttle Comprising One Fusion Protein of AGT with FKBP, One Fusion Protein of ACT with MEK 1 and Fluorescein as Cargo

A microplate is modified by absorbing an equimolar mixture of recombinantly expressed FRB with a polyhistidine tag and recombinantly expressed ERK2 with a polyhistidine tag. The human gene of ERK2 (extracellular signal regulated kinase 2) is obtained from RZPD, Heidelberg, Germany. A range of densities is established according to standard ELISA protocols. After washing and blocking according to standard ELISA protocols, the shuttle comprising one FKBP fusion protein and one MEK1 fusion protein of Example 60 is incubated on this surface. For comparison a structure containing just one of each binder molecule and a fluorescent compound is incubated in another well. By systematic variation of the concentration of the complete shuttle structure the rough affinity is estimated. The affinity of the complete shuttle structure is at least two times higher than that of the individual interactions alone.

### Example 63

### Binding Properties of a Molecular Shuttle Comprising Three Fusion Proteins of AGT with FKBP and Fluorescein as Cargo on Living Cells

A variant of the FRB, for expression as a membrane resident target protein, was constructed by combining (from N-terminus to C-terminus) the signal sequence of the 5HT3 receptor, followed by the gene of FRB, followed by the single transmembrane domain of the human transferrin receptor and cloning this into a mammalian expression vector. Cells transfected with an expression plasmid encoding this membrane bound FRB on the outer surface of the cell membrane are incubated either with a single binder protein modified with a fluorophore or with the shuttle comprising three fusion proteins of AGT with FKBP of Example 58. Both experiments are done with the same molecular concentration of the monomeric or the trimeric binder (shuttle of Example 58). After 10 and 30 minutes of incubation the cells are washed and the internalised fluorophore is estimated from fluorescence micrographs. A background correction is done for the amount of fluorophore internalised without the receptor structure being expressed on the cells. The amount of fluorophore that is internalised and cannot be washed away for the shuttle of Example 58 is at least two times as high as for the monomeric binder molecule.

### Example 64

### Binding Properties of a Molecular Shuttle Comprising Three Fusion Proteins of AGT with FKBP and Both Chlorambucil and Fluorescein as Cargo on Living Cells

Cells transfected with an expression plasmid encoding for membrane bound FRB (see Example 63) on the outer surface of the cell membrane are incubated either with a single binder protein modified with a fluorophore or with the trimeric binder (shuttle of Example 59). Both experiments are done with the same molecular concentration of the monomeric binder protein or the shuttle. After 10 and 30 minutes of incubation the cells are washed and the internalised fluorophore is estimated from fluorescence micrographs. A background correction is done for the amount of fluorophore internalised without the receptor structure being expressed on the cells. The amount of fluorophore that is internalised and cannot be washed away, is at least two times as high for the trimeric binder structure than for the monomeric binder molecule.

### Example 65

### Properties of a Molecular Shuttle Comprising Three Fusion Proteins of AGT with FKBP and Both siRNA and Fluorescein as Cargo on Living Cells

Cells transfected with an expression plasmid encoding for membrane bound FRB on the outer surface of the cell membrane (see Example 63) are incubated either with a single binder protein modified with a fluorophore or with a shuttle carrying one molecule of fluorescein and one molecule of a siRNA as described in Example 39. The siRNA is selected against SNAP26 from Covalys. The effect of the siRNA on the expression of the SNAP-tag is tested 2 h, 8 h, and 24 h after incubation of the cells. During the incubation all preexisting SNAP-tag protein is blocked during the incubation step by a transient incubation with 10 μM benzylguanine. Free siRNA and siRNA bound to the homotrimeric binder molecule carrying siRNA and a fluorophore of Example 39 are compared. The expression level of the SNAP26 target protein is at its minimum at least reduced by 50%.

### Example 66

### Binding Properties of a Molecular Shuttle Comprising One Fusion Protein of AGT with FKBP, and One Fusion Protein of ACT with MEK1, and Both Chlorambucil and Fluorescein as Cargo on Living Cells

A variant of the ERK2, for expression as a membrane resident target protein, is constructed by combining (from N-terminus to C-terminus) the signal sequence of the 5HT3 receptor, followed by the gene of ERK2, followed by the single transmembrane domain of the human transferrin receptor and cloning this into a mammalian expression vector. Cells transfected with the corresponding genes and expressing both the target proteins (membrane bound FRB, see Example 63, and membrane bound ERK2) on the outer surface of the cell membrane are incubated either with a single enzyme-binder protein modified with the corresponding fluorescein substrate or with the shuttle described in Example 60. All experiments are done with the same molecular concentration of the monomeric or the heterodimeric binder. After 10 and 30 minutes of incubation the cells are washed and the internalised fluorescein is estimated from fluorescence micrographs. A background correction is done for the amount of fluorescein internalised without any of the receptor structures being expressed on the cells. The amount of fluorescein that is internalised and cannot be washed away is at least two times as high for the heterodimeric binder structure than for the monomeric binder molecule.

### Example 67

### BG-PEG12-NHFmoc (79)

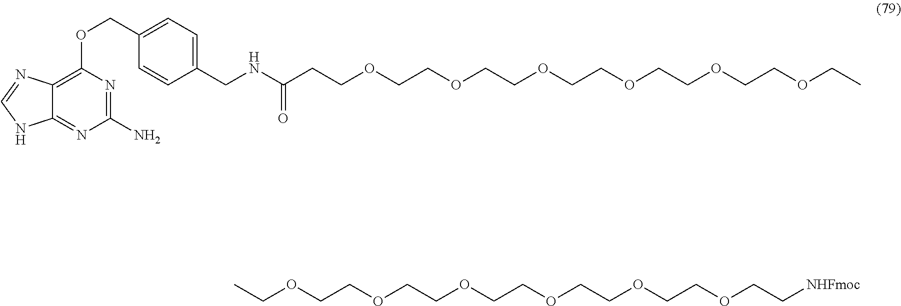

To a solution of Fmoc-amido-PEG12-acid (1 g, 1.19 mmol) in DMF (2 mL) PYBOP (619 mg, 1.19 mmol) is added at rt. The solution is stirred at rt for 20 min. O⁶-(4-aminomethyl)benzyl guanine (320 mg, 1.19 mmol) and DIPEA (196 μL, 1.19 mmol) are added and the solution is heated at 50° C. for 5 min. The solution is stirred at rt overnight. The crude mixture is poured into diethyl ether. The precipitate is collected and washed with diethyl ether. The obtained solid is dissolved in methanol and the solvent is concentrated until dryness. No further purification is required. MS (ESI) m/z 1093 [M+H]⁺.

### Example 68

### BG-PEG12-NH₂ (80)

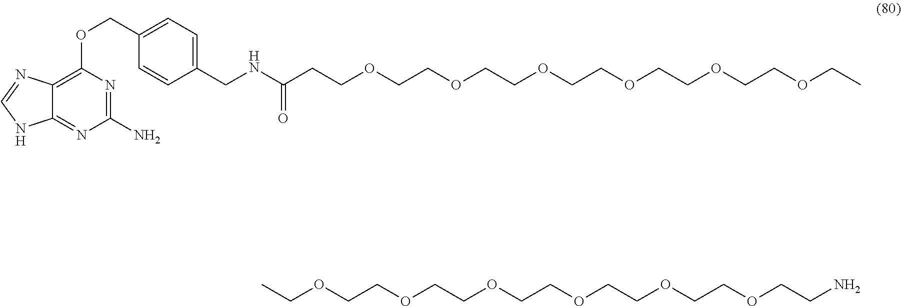

To a solution of compound (79) (1.5 g, 1.72 mmol) in dioxane (10 mL) diethylamine (2.5 mL) is added at rt. The solution is stirred at rt for 3 h. The solvent is removed under reduced pressure. The crude mixture is dissolved in DMF (1.5 mL) and poured into diethyl ether (10 mL). The resulting precipitate is collected. No further purification is required. MS (ESI) m/z 871 [M+H]⁺.

### Example 69

### Tris{[2-carboxyethoxy]methyl}methylamine (81)

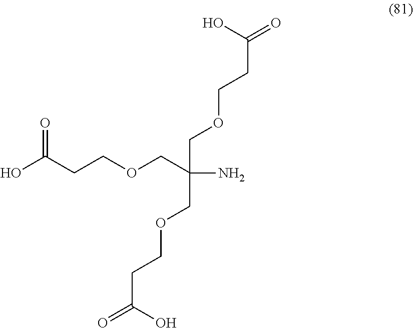

Tris{[2-tert-butoxycarbonyl)ethoxy]methyl}methylamine (1) (4.3 g, 8 mmol) is stirred in 80 mL of 96% formic acid for 18 h. Formic acid is removed at reduced pressure at 50° C. to produce a colorless oil in quantitative yield. ¹H NMR ((CD₃)₂SO, 400 MHz): 8.2 (m, 2H), 7.45 (m, 3H), 3.6 (m, 6H), 3.4 (m, 6H), 2.45 (m, 6H).

### Example 70

### N-Tris[(2-carboxyethoxy)methyl]methyl 7-(diethylamino)coumarin-3-carboxamide (82)

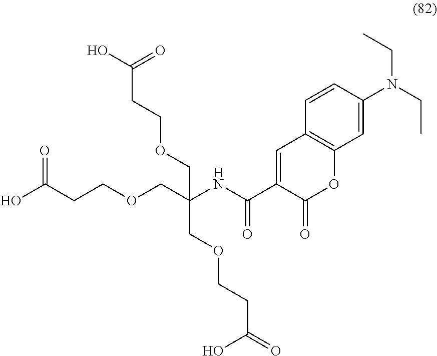

Compound (81) (66 mg, 0.195 mmol) and 7-(diethylamino)coumarin-3-carboxylic acid N-succinimidyl ester (70 mg, 0.195 mmol) are dissolved in 2 mL of DMF with Et₃N (28 μL, 0.195 mmol) and heated overnight at 40° C. The solvent is evaporated under vacuum and compound (82) isolated by reversed phase HPLC on a C18 column using a linear gradient of water:acetonitrile (from 95:5 to 20:80 in 20 min, 0.08% TFA). MS (ESI) m/z 581 [M+H]⁺.

### Example 71

### N-Tris{[2-(tert-butoxycarbonyl)ethoxyl]methyl}methyl ATTO-495-carboxamide (83)

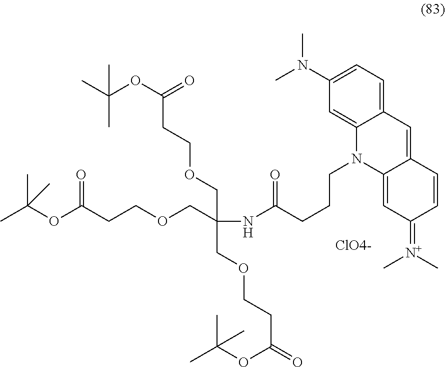

Tris{[2-(tert-butoxycarbonyl)ethoxy]methyl}methylamine (1) (4.8 mg, 9.45 μmol) and ATTO-495 N-succinimidyl ester (5.2 mg, 9.45 μmol) are dissolved in 2 mL DMF with Et₃N (1.3 μL, 9.45 μmol) and heated overnight at 40° C. The solvent is evaporated under vacuum and compound (83) isolated by reversed phase HPLC on a C18 column using a linear gradient of water:acetonitrile (from 95:5 to 20:80 in 20 min, 0.08% TFA).

MS (ESI) m/z 841 [M+H]⁺.

### Example 72

### N-Tris[(2-carboxyethoxy)methyl]methyl ATTO-495-carboxamide (84)

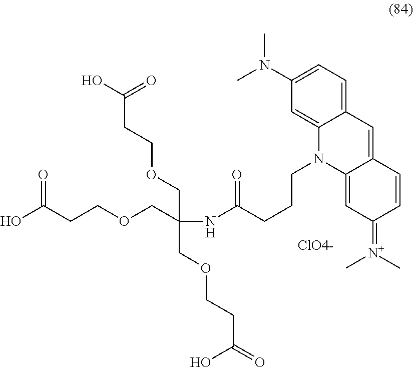

Compound (83) (210 mg, 0.25 mmol) is stirred in 250 μL of 96% formic acid for 18 h. Formic acid is removed at reduced pressure at 5° C. to produce a colorless oil in quantitative yield. MS (ESI) m/z 672 [M+H]⁺.

### Example 73

### N-Tris{[2-(tert-butoxycarbonyl)ethoxy]methyl}methyl nile red-oxyacetamide (85)

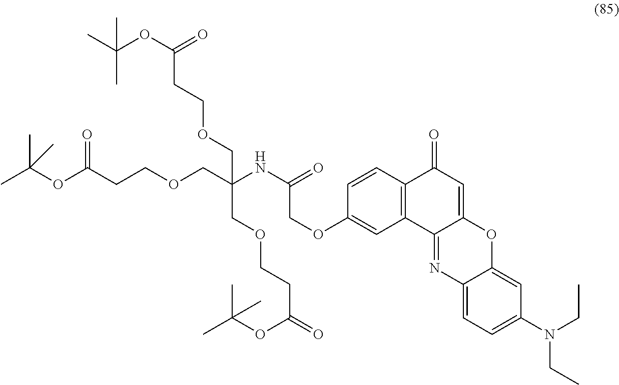

To a solution of nile red-oxyacetic acid (9-diethylamino-5-oxo-benzo[a]phenoxazin-2-oxyacetic acid, 100 mg, 0.255 mmol, 1 eq) in DMF (50 mL) are successively added DCC (160 mg, 0.765 mmol, 3 eq) and NHS (90 mg, 0.765 mmol, 3 eq). The resulting mixture is stirred overnight. Then DCU salts are removed by centrifugation. Compound (1) (130 mg, 0.255 mmol, 1 eq) and DIPEA (42 μL, 0.255 mmol, 1 eq) are added to the solution at rt. The resulting mixture is stirred overnight. The solvent is removed under reduced pressure. Flash chromatography (CH₂Cl₂/methanol, 10/1→5/1) gives the desired compound (85). MS (ESI) m/z 881 [M+H]⁺.

### Example 74

### N-Tris[(2-carboxyethoxy)methyl]methyl nile red-oxyacetamide (86)

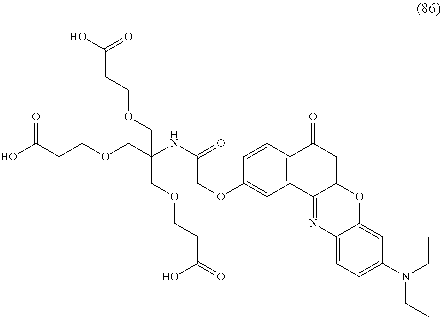

Compound (85) (70 mg, 0.08 mmol) is stirred in 250 μL of 96% formic acid for 18 h. Formic acid is removed under reduced pressure at 50° C. to produce a colorless oil in quantitative yield. MS (ESI) m/z 712 [M+H]⁺.

### Example 75

### N-Tris{[2-(tert-butoxycarbonyl)ethoxy]methyl}methyl 5-maleimidopentane-carboxamide (87)

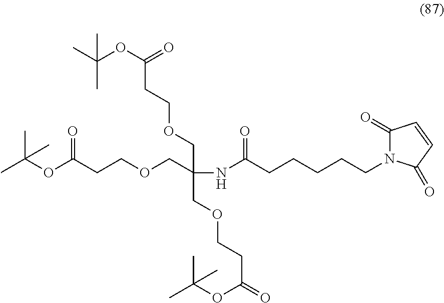

To a solution of 6-maleimido-hexanoic acid (106 mg, 0.5 mmol) in DMF (5 mL) PYBOP (260 mg, 0.5 mmol) is added at rt. The solution is stirred at rt for 20 min. Compound (1) (253 mg, 0.5 mmol) and DIPEA (83 μL, 0.5 mmol) are added and the solution is heated at 50° C. for 5 min. The solution is stirred at rt overnight. The solvent is removed under reduced pressure. Flash chromatography (cyclohexane/ethyl acetate, 1/1) gives the desired compound (87). ¹H NMR ((CD₃)₂SO, 400 MHz): 6.7 (s, 2H), 3.7 (s, 6H), 3.65 (m, 6H), 3.5 (m, 2H), 2.45 (m, 6H), 2.1 (m, 2H), 1.6 (m, 4H), 1.45 (m, 27H), 1.35 (m, 2H).

### Example 76

### N-Tris[(2-carboxyethoxy)methyl]methyl 5-maleimidopentanecarboxamide (88)

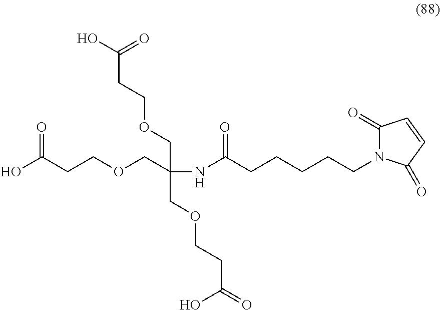

Compound (87) (214 mg, 0.305 mmol) is stirred in 3 mL of 96% formic acid for 18 h. Formic acid is removed at reduced pressure at 50° C. to produce a colorless oil in quantitative yield. The compound is directly used for next step.

### Example 77

### N-Tris-{[2-(BG-PEG12-NH-carbonyl)ethoxy]methyl}methyl 7-(diethylamino)-coumarin-3-carboxamide (89)

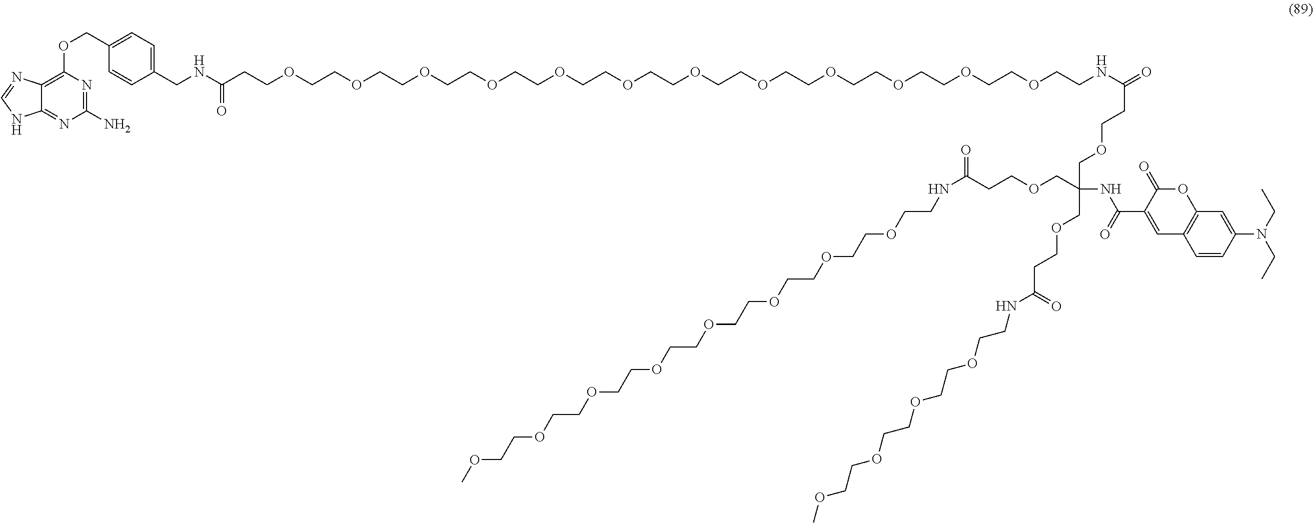

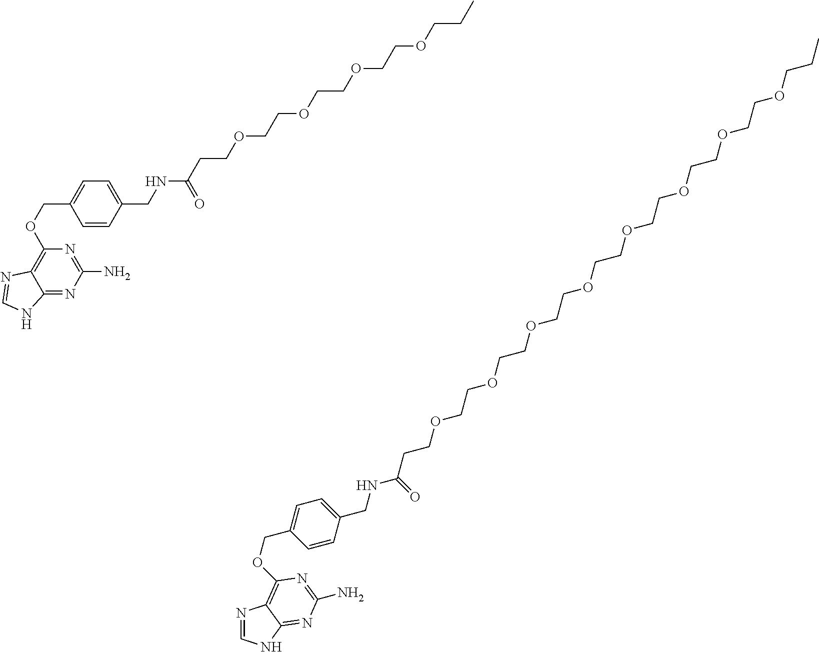

To a solution of N-Tris[(2-carboxyethoxy)methyl]methyl 7-(diethylamino)coumarin-3-carboxamide (82) (10 mg, 0.018 mmol) and BG-PEG12-NH₂ (80) (54 mg, 0.062 mmol, 3.6 eq) in DMF (1 mL) are successively added DIPEA (8 μL, 0.062 mmol, 3.6 eq), HOBT (1 M in NMP, 18 μL, 0.018 mmol, 1 eq) and EDC (12 mg, 0.062 mmol, 3.6 eq) at rt. The resulting mixture is stirred overnight. The solvent is evaporated under vacuum and compound (89) isolated by reversed phase HPLC on a C18 column using a linear gradient of water:acetonitrile (from 95:5 to 20:80 in 20 min, 0.08% TFA). The structural ability of compound (89) to trigger the formation of a protein trimer is confirmed by in vitro experiments using the fusion protein SNAP-FKBP according to Example 85. The formation of the protein trimer is visualized by SDS-PAGE followed by coomassie staining of the proteins.

### Example 78

### N-Tris-{[2-(BG-PEG12-NH-carbonyl)ethoxy]methyl}methyl ATTO-495-carboxamide (90)

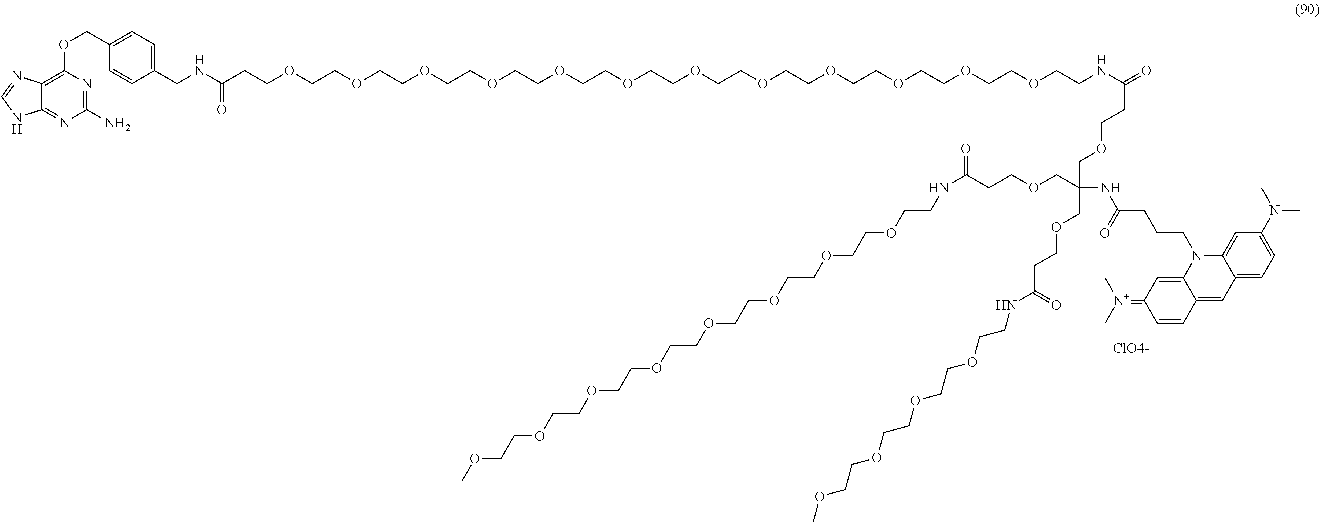

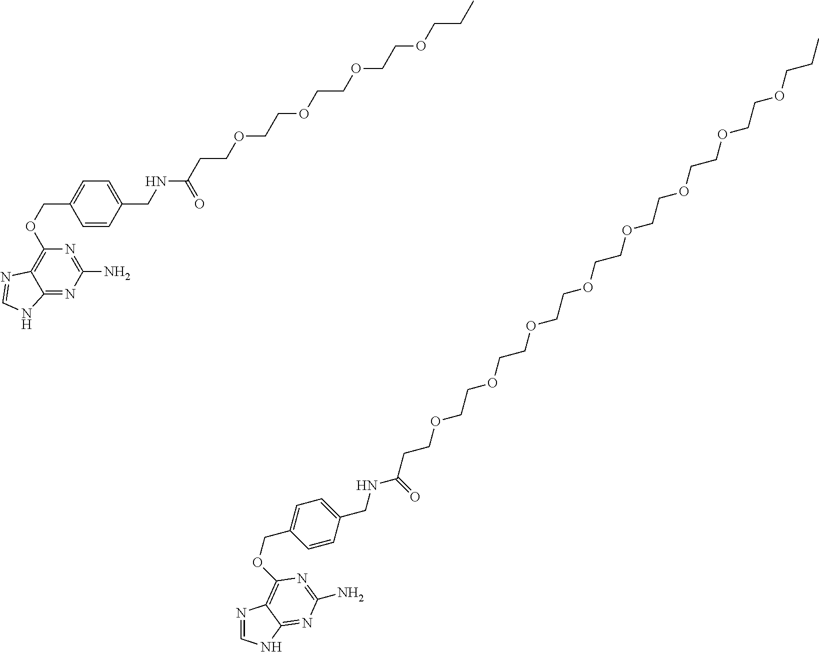

To a solution of N-Tris[(2-carboxyethoxy)methyl]methyl ATTO-495-carboxamide (84) (4 mg, 0.005 mmol) and BG-PEG12-NH₂ (80) (15 mg, 0.0175 mmol, 3.6 eq) in DMF (1 mL) are successively added DIPEA (3 μL, 0.0175 mmol, 3.6 eq), HOBT (1 M in NMP, 5 μL, 0.005 mmol, 1 eq) and EDC (4 mg, 0.0175 mmol, 3.6 eq) at rt. The resulting mixture is stirred overnight. The solvent is evaporated under vacuum and compound (90) isolated by reversed phase HPLC on a C18 column using a linear gradient of water:acetonitrile (from 95:5 to 20:80 in 20 min, 0.08% TFA). The structural ability of compound (90) to trigger the formation of a protein trimer is confirmed by in vitro experiments using the fusion protein SNAP-FKBP according to Example 85.

### Example 79

### N-Tris-{[2-(BG-PEG12-NH-carbonyl)ethoxy]methyl}methyl nile red-oxyacetamide (91)

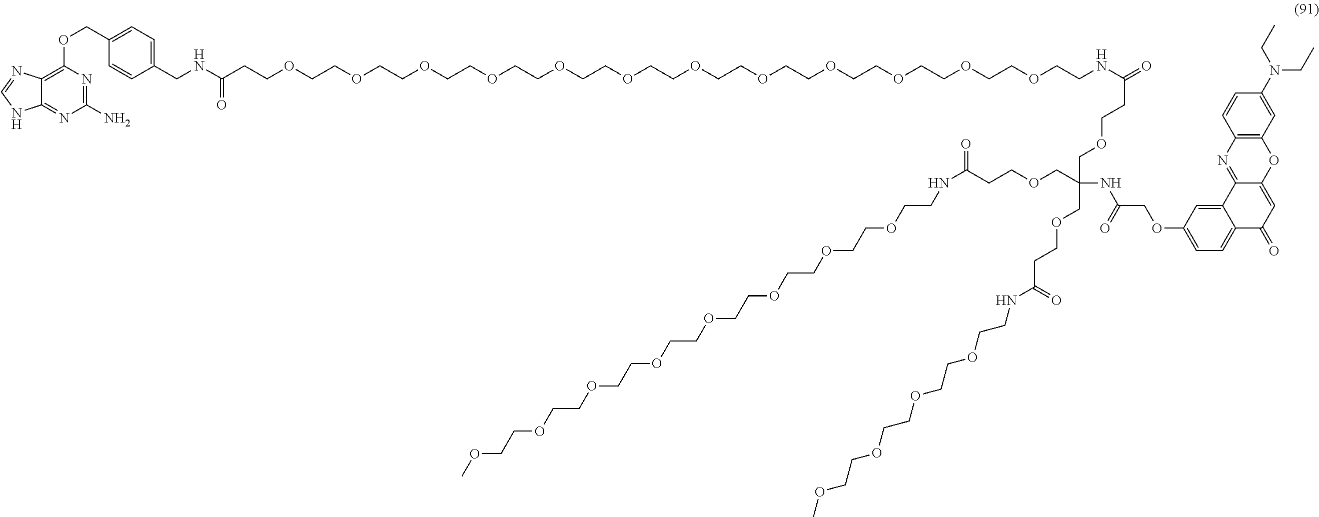

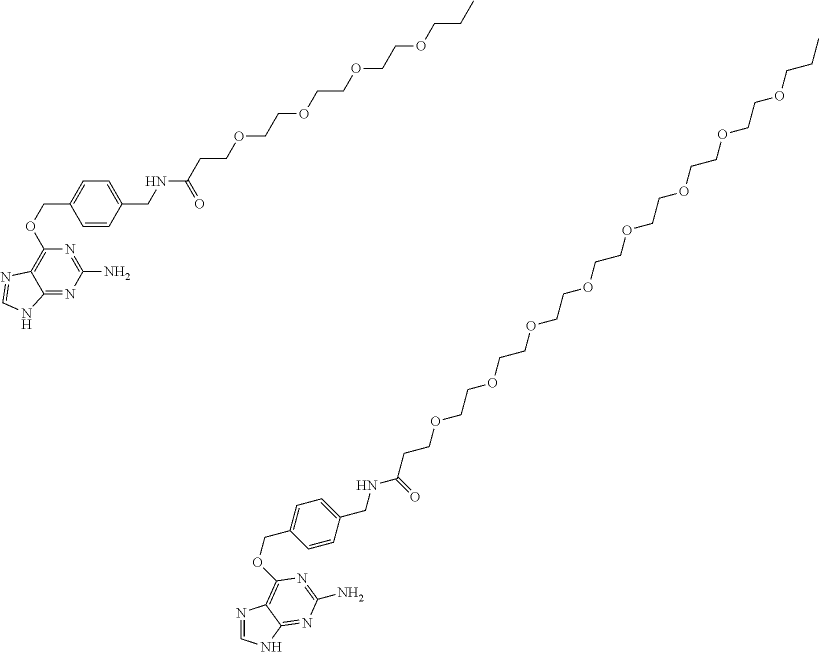

To a solution of N-tris[(2-carboxyethoxy)methyl]methyl nile red-oxyacetamide (86) (8 mg, 0.011 mmol) and BG-PEG12-NH₂ (80) (34 mg, 0.039 mmol, 3.6 eq) in DMF (1 mL) are successively added DIPEA (7 μL, 0.039 mmol, 3.6 eq), HOBT (1 M in NMP, 11 μL, 0.01 mmol, 1 eq) and EDC (8 mg, 0.039 mmol, 3.6 eq) at rt. The resulting mixture is stirred overnight. The solvent is evaporated under vacuum and the compound (91) isolated by reversed phase HPLC on a C18 column using a linear gradient of water:acetonitrile (from 95:5 to 20:80 in 20 min, 0.08% TFA). The structural ability of compound (91) to trigger the formation of a protein trimer is confirmed by in vitro experiments using the fusion protein SNAP-FKBP according to Example 85.

### Example 80

### N-Tris-{[2-(BG-PEG12-NH-carbonyl)ethoxy]methyl}methyl 5-maleimido-pentanecarboxamide (92)

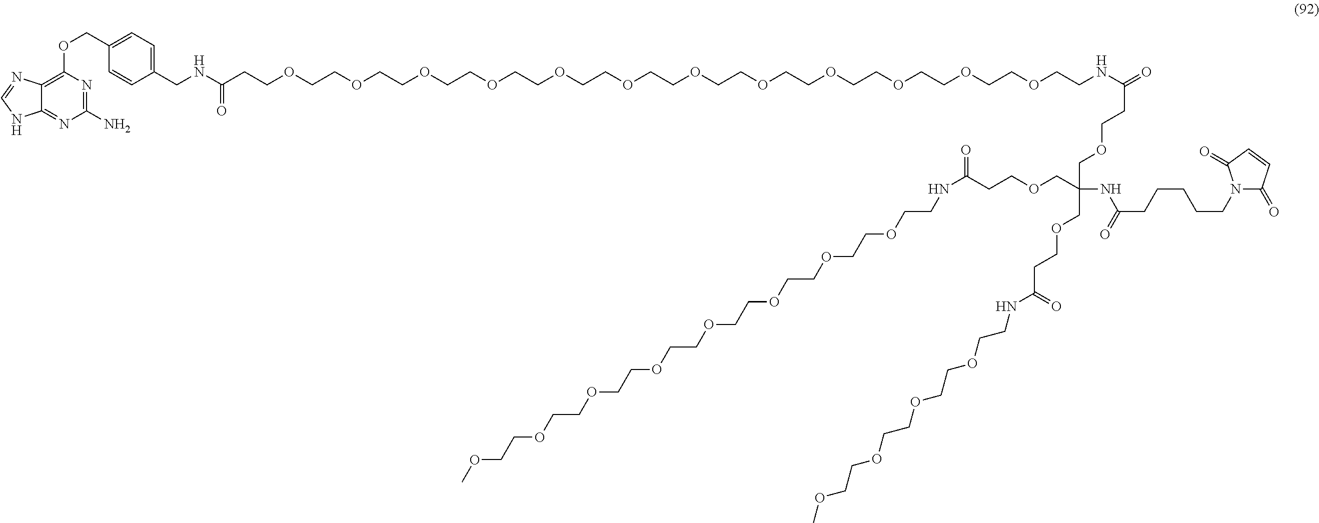

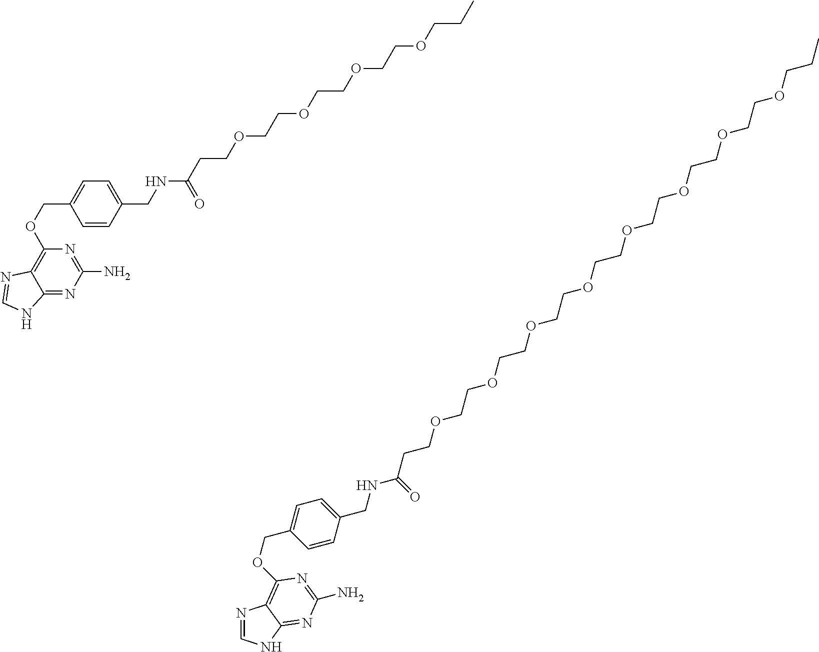

To a solution of N-tris[(2-carboxyethoxy)methyl]methyl 5-maleimidopentanecarboxamide (88) (8 mg, 0.016 mmol) and BG-PEG12-NH₂ (80) (50 mg, 0.057 mmol, 3.6 eq) in DMF (1 mL) are successively added DIPEA (10 μL, 0.057 mmol, 3.6 eq), HOBT (1 M in NMP, 16 μL, 0.016 mmol, 1 eq) and EDC (2 mg, 0.057 mmol, 3.6 eq) at rt. The resulting mixture is stirred overnight. The solvent is evaporated under vacuum and the compound (92) isolated by reversed phase HPLC on a C18 column using a linear gradient of water:acetonitrile (from 95:5 to 20:80 in 20 min, 0.08% TFA). The structural ability of compound (92) to trigger the formation of a protein trimer is confirmed by in vitro experiments using the fusion protein SNAP-FKBP according to Example 85.

### Example 81

### 3-[2-(2-Maleimidoethyl)disulfanyl]propanoic acid (93)

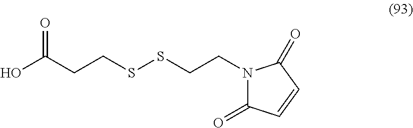

A solution of 3-[2-(2-aminoethyl)disulfanyl]propanoic acid (250 mg, 1.38 mmol) and maleic anhydride (272 mg, 2.76 mmol) in a mixture of acetic acid/toluene (3/1, 3 mL) is heated overnight at 120° C. The crude mixture is cooled to rt, and further cooled in an ice bath to 0° C. Pentane (50 mL) is added, and a precipitate is formed. Diethyl ether is added to this precipitate, and the white solid formed is removed. The ether solution is concentrated under vacuum to yield the product (93). No further purification is required. ¹H NMR ((CD₃)₂SO, 400 MHz): 7.4 (s, 1H), 6.7 (s, 2H), 3.7 (m, 2H), 2.9 (m, 4H), 2.6 (m, 2H).

### Example 82

### N-Tris{[2-(tert-butoxycarbonyl)ethoxy]methyl}methyl 3-[2-(2-maleimido-ethyl)disulfanyl]propanoylamide (94)

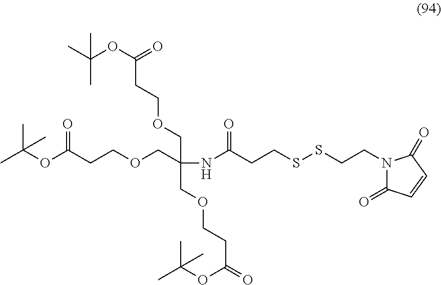

To a solution of 3-[2-(2-maleimidoethyl)disulfanyl]propanoic acid (93) (188 mg, 0.72 mmol) in DMF (2 mL) PYBOP (376 mg, 0.72 mmol) is added at rt. The solution is stirred at rt for 20 min. Tris{[2-tert-butoxycarbonyl)ethoxy]methyl}methylamine (1) (364 mg, 0.72 mmol) and DIPEA (119 μL, 0.72 mmol) are added and the solution is heated at 50° C. for 5 min. The solution is stirred at rt overnight. The solvent is removed under reduced pressure. Flash chromatography (cyclohexane/ethyl acetate, 2/1) gives the desired compound (94). ¹H NMR ((CD₃)₂SO, 400 MHz): 6.6 (s, 2H), 3.8 (m, 2H), 3.6 (m, 6H), 3.55 (m, 6H), 2.8 (m, 4H), 2.5 (m, 2H), 2.35 (m, 6H), 1.4 (m, 27H).

### Example 83

### N-Tris[(2-carboxyethoxy)methyl]methyl 3-[2-(2-maleimidoethyl)disulfanyl]-propanoylamide (95)

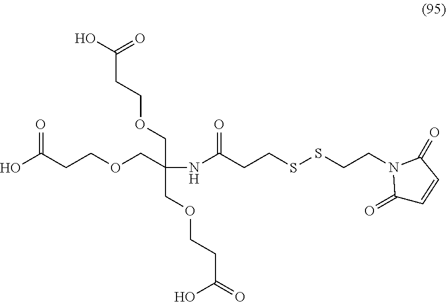

Compound (94) (112 mg, 0.15 mmol) is stirred in 1.5 mL of 96% formic acid for 18 h. Formic acid is removed at reduced pressure at 50° C. to produce a colorless oil in quantitative yield. The compound is directly used for the next step. ¹H NMR ((CD₃)₂SO, 400 MHz): 7.0 (s, 2H), 3.7 (m, 2H), 3.55 (m, 12H), 2.75 (m, 4H), 2.45 (m, 6H).

### Example 84

### N-Tris-{[2-(BG-PEG12-NH-carbonyl)ethoxy]methyl}methyl 3-[2-(2-maleimidoethyl)disulfanyl]propanoylamide (96)

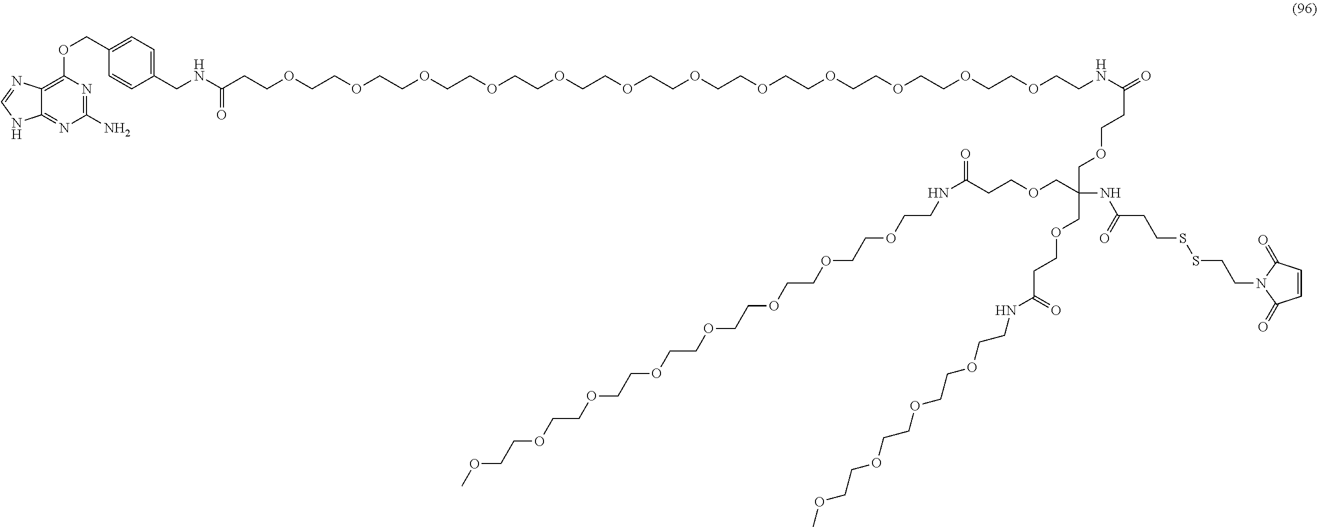

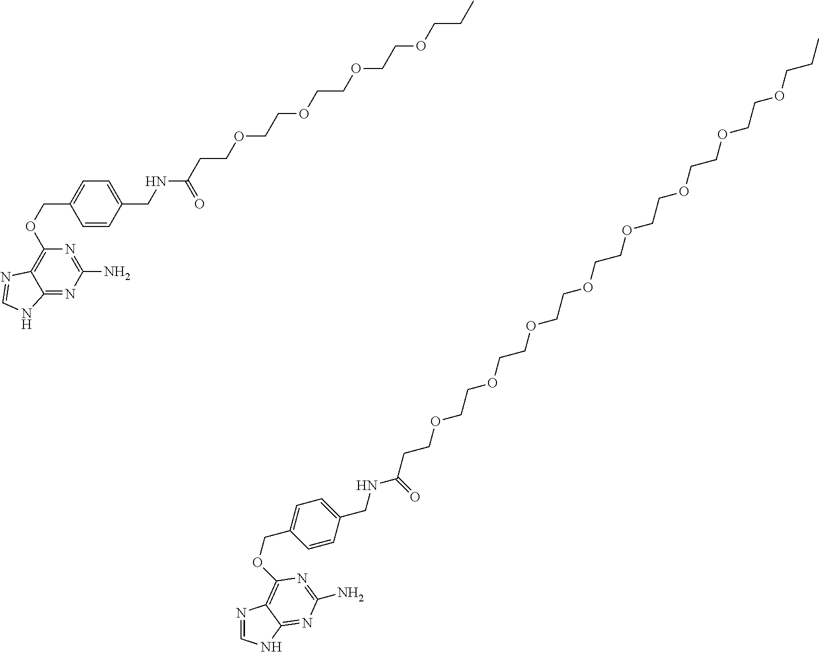

To a solution of compound (95) (10 mg, 0.017 mmol) and BG-PEG12-NH₂ (80) (120 mg, 0.138 mmol, 8 eq) in DMF (1 mL) are successively added DIPEA (17 μL, 0.069 mmol, 4 eq), HOBT (1 M in NMP, 17 μL, 0.017 mmol, 1 eq) and EDC (14 mg, 0.069 mmol, 4 eq) at rt. The resulting mixture is stirred overnight. The solvent is evaporated under vacuum and the compound (96) isolated by reversed phase HPLC on a C18 column using a linear gradient of water:acetonitrile (from 95:5 to 20:80 in 20 min, 0.08% TFA). The structural ability of compound (96) to trigger the formation of a protein trimer is confirmed by in vitro experiments using the fusion protein SNAP-FKBP according to Example 85.

### Example 85

### Determination of the Reactivity of Compounds (89), (90), (91), (92) and (96) with FKBP-AGT Fusion Protein

1 μL of a 591 μM solution of FKBP protein fused to a variant of AGT available from Covalys as SNAP26 and 1 μL of a 100 μM solution of compound (89), (90), (91), (92) or (96) are added to 8 μL of a solution of 50 mM Tris-HCl pH 7.5; 100 mM NaCl; 0.1% Tween20; 1 mM DTT. Following a 4 h incubation at rt, 15 μL of a solution of 100 mM Tris-HCl pH 6.8; 2% SDS; 35% glycerol; 10 mM EDTA; 20 mM DTT is added. The mixture is boiled for 5 min at 95° C. After cooling to rt, 25 μL of this solution is loaded on a 4-20% linear gradient SDS-PAGE gel. After electrophoresis, the proteins are coomassie stained in gel to visualize protein trimer.

### Example 86

### BC-PEG12-NHFmoc (97)

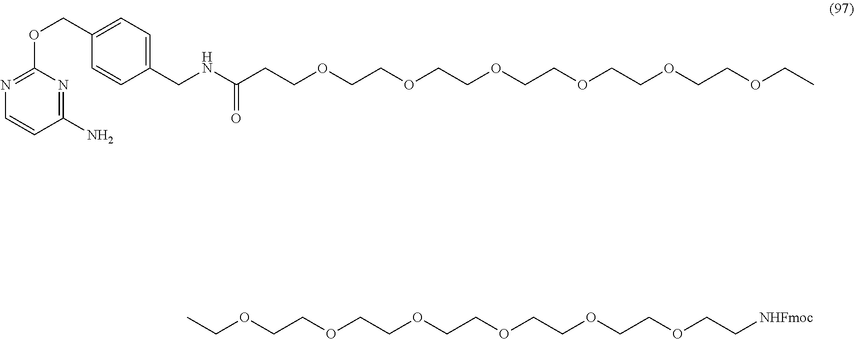

To a solution of Fmoc-amido-PEG12-acid (250 g, 0.3 mmol) in DMF (2 mL) PYBOP (155 mg, 0.3 mmol) is added at rt. The solution is stirred at rt for 20 min. BC—NH2 (2-(4-aminomethylbenzyloxy)-4-aminopyrimidine=aminomethylbenzylcytosine, 69 mg, 0.3 mmol) and DIPEA (49 μL, 0.3 mmol) are added and the solution is heated at 50° C. for 5 min. The solution is stirred at rt overnight. The crude mixture is poured into diethyl ether. The precipitate is collected and washed with diethyl ether. The obtained solid is dissolved in methanol and the solvent is concentrated until dryness. No further purification is required. MS (ESI) m/z 1053 [M+H]⁺.

### Example 87

### BC-PEG12-NH2 (98)

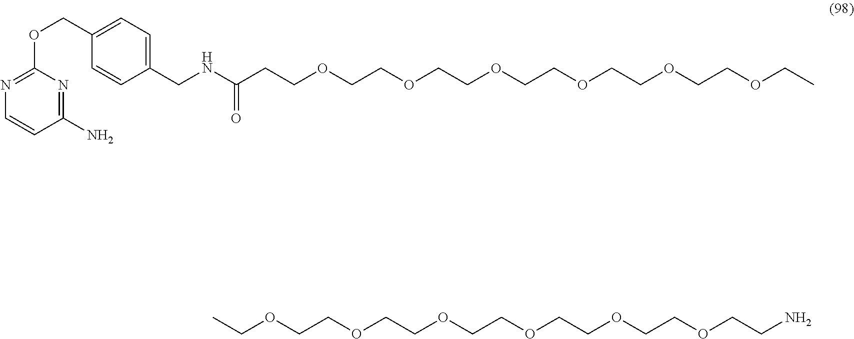

To a solution of compound (97) (320 mg, 0.3 mmol) in DMF (1.5 mL) diethylamine (300 μL) is added at rt. The solution is stirred at rt for 3 h. The solvent is removed under reduced pressure. The crude mixture is dissolved in DMF (1.5 mL) and poured into diethyl ether (10 mL). The resulting precipitate is collected. No further purification is required. MS (ESI) m/z 830 [M+H]⁺.

### Example 88

### N-Tris-{[2-(BC-PEG12-NH-carbonyl)ethoxyl]methyl}methyl nile red-oxyacetamide (99)

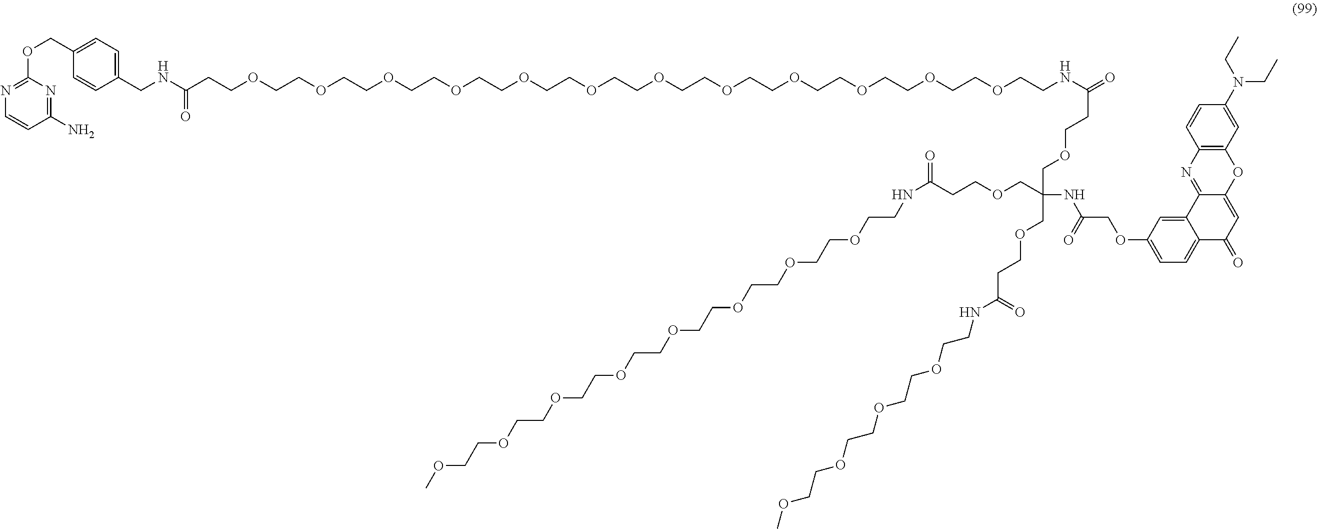

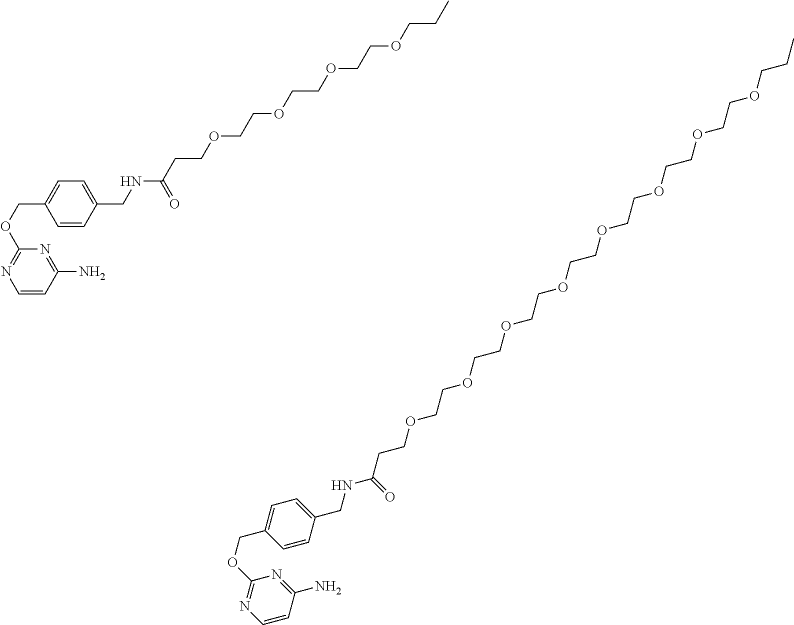

To a solution of N-tris[(2-carboxyethoxy)methyl]methyl nile red-oxyacetamide (86) (10 mg, 0.014 mmol) and BC-PEG12-NH₂ (98) (41 mg, 0.049 mmol, 3.5 eq) in DMF (1 mL) are successively added DIPEA (8.1 μL, 0.049 mmol, 3.5 eq), HOBT (1 M in NMP, 14 μL, 0.014 mmol, 1 eq) and EDC (10 mg, 0.049 mmol, 3.5 eq) at rt. The resulting mixture is stirred overnight. The solvent is evaporated under vacuum and compound (99) isolated by reversed phase HPLC on a C18 column using a linear gradient of water:acetonitrile (from 95:5 to 20:80 in 20 min, 0.08% TFA). The structural ability of compound (99) to trigger the formation of a protein trimer is confirmed by in vitro experiments using the fusion protein CLIP-FRB. 20 μL of a 52.9 μM solution of FRB protein fused to a variant of AGT available from Covalys under the trade name CLIP and 2 μL of a 100 μM solution of compound (99) is added to 3 μL of a solution of 50 mM Iris-HCl pH 7.5; 100 mM NaCl; 0.1% Tween20; 1 mM DTT. Following a 4 h incubation at rt, 15 μL of a solution of 100 mM Tris-HCl pH 6.8; 2% SDS; 35% glycerol; 10 mM EDTA; 20 mM DTT is added. Then the mixture is boiled for 5 min at 95° C. After cooling to rt, 25 μL of this solution is loaded on a 4-20% linear gradient SDS-PAGE gel. After electrophoresis, the proteins are coomassie stained in gel to visualize protein trimer.

### Example 89

### N-Tris-{[2-(halo-PEG4-NH-carbonyl)ethoxyl]methyl}methyl nile red-oxyacetamide (100)

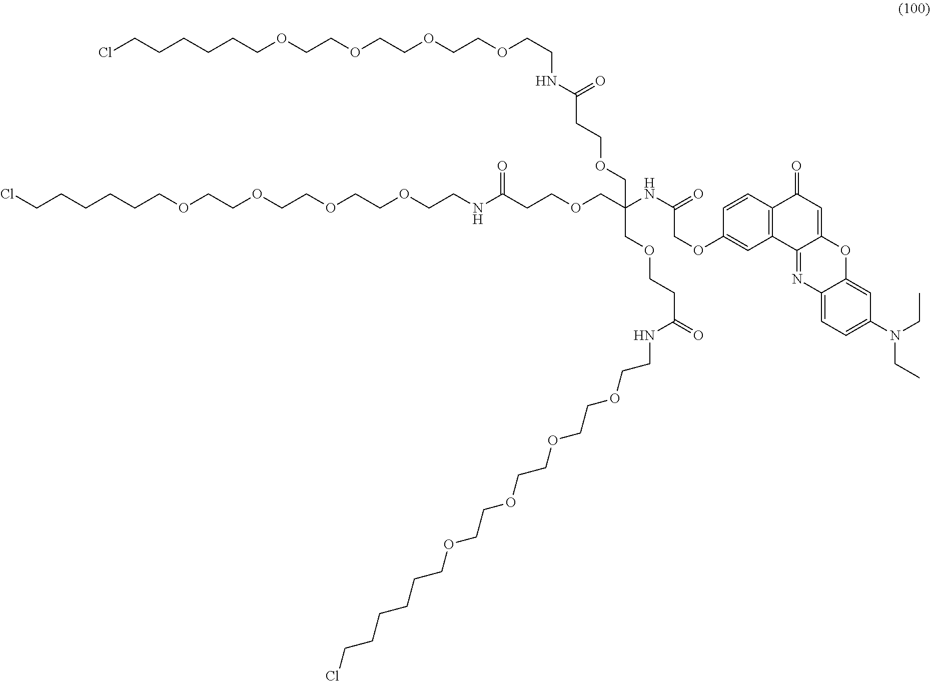

To a solution of N-tris[(2-carboxyethoxy)methyl]methyl nile red-oxyacetamide (86) (6 mg, 0.0083 mmol) and 18-chloro-3,6,9,12-tetraoxaoctadecan-1-amine (10 mg, 0.029 mmol, 3.5 eq) in DMF (1 mL) are successively added pyridine (5 μL, 0.058 mmol, 7 eq), HOBT (1 M in NMP, 8.3 μL, 0.0083 mmol, 1 eq) and EDC (6 mg, 0.029 mmol, 3.5 eq) at rt. The resulting mixture is stirred overnight. The solvent is evaporated under vacuum and compound (100) isolated by reversed phase HPLC on a C18 column using a linear gradient of water:acetonitrile (from 95:5 to 20:80 in 20 min, 0.08% TFA). The structural ability of compound (100) to trigger the formation of a protein trimer is confirmed by in vitro experiments using the fusion protein H2. 10 μL of a 70 μM solution of HaloTag protein available from Promega Corporation and 1.8 μL of a 230 μM solution of compound (100) is added to 3.2 μL of a solution of 50 mM Tris-HCl pH 7.5; 100 mM NaCl; 0.1% Tween20; 1 mM DTT. Following a 4 h incubation at rt, 15 μL of a solution of 100 mM Tris-HCl pH 6.8; 2% SDS; 35% glycerol; 10 mM EDTA; 20 mM DTT is added. Then the mixture is boiled for 5 min at 95° C. After cooling to rt, 25 μL of this solution is loaded on a 4-20% linear gradient SDS-PAGE gel. After electrophoresis, the proteins are coomassie stained in gel to visualize protein trimer.

### Example 90

### 2-Phthalimido-N-(BG)-succinic acid monoamide (101)

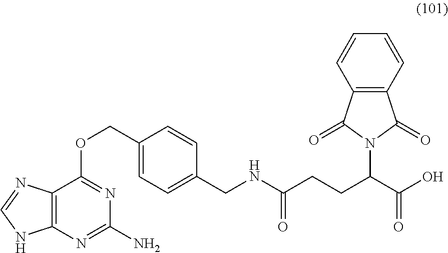

To a solution of BG-NH2 (135 mg, 0.5 mmol, 1 eq) in DMF (2 mL) 2-phthalimido-succinic anhydride (130 mg, 0.5 mmol, 1 eq) is added at rt. The reaction mixture is stirred at rt for 4 h, then the crude mixture is poured into water (40 mL). The pH of the water phase is adjusted to pH 8 with NaOH (1 M), and the precipitate disappears. The aqueous layer is washed with ethyl acetate (2 times 100 mL). Then the pH is adjusted to pH 4 and the precipitate is collected. ESI-MS: m/z 530 [M+H]⁺.

### Example 91

### N5-{4-[(2-amino-9H-purin-6-yloxy)methyl]benzyl}-N1-{4-[(4-aminopyrimidin-2-yloxy)methyl]benzyl}-2-(1,3-dioxoisoindolin-2-yl)-pentanediamide (102)

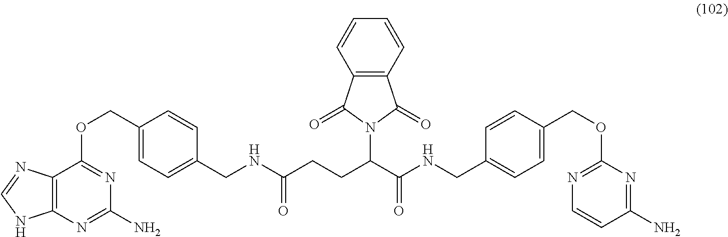

To a solution of (101) (130 mg, 0.245 mmol) in DMF (2 mL) PYBOP (128 mg, 0.245 mmol) is added at rt. The solution is stirred at rt for 20 min. BC—NH2 (56 mg, 0.245 mmol) and DIPEA (42 μL, 0.245 mmol) are added and the solution is heated at 50° C. for 5 min. The solution is stirred at rt overnight. The solvent is removed under reduced pressure, and the compound is obtained after precipitation in water.

ESI-MS: m/z 764 [M+Na]⁺.

### Example 92

### 2-Amino-N-5-{4-[(2-amino-9H-purin-6-yloxy)methyl]benzyl}-N-1-{4-[(4-aminopyrimidin-2-yloxy)methyl]benzyl}-pentanediamide (103)

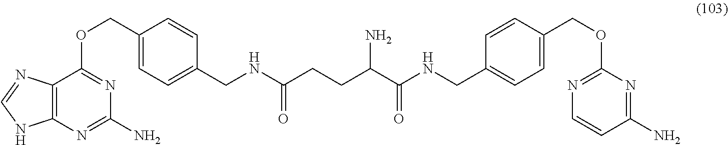

To a solution of compound (102) (70 mg, 0.094 mmol) in methanol (1 mL) methylamine (33% in ethanol, 2 mL) is added, and the solution is stirred at rt for 12 h. The solvent is removed under reduced pressure and compound (103) is obtained by precipitation of the crude mixture in diethyl ether. ESI-MS: m/z 656 [M+HCOOH]⁺.

### Example 93

### N5-{4-[(2-amino-9H-purin-6-yloxy)methyl]benzyl}-N1-{4-[(4-aminopyrimidin-2-yloxy)methyl]benzyl}-2-tetramethylrhodamine-5-carboxamido-pentanediamide (104) and corresponding tetramethylrhodamine-6-carboxamido compound (105)

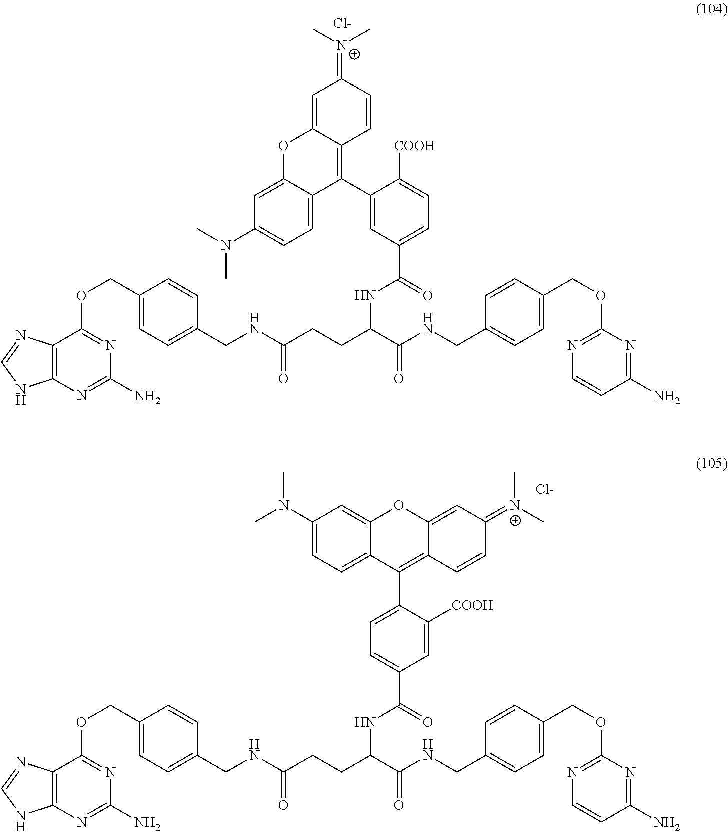

Compound (103) (2.4 mg, 0.0039 mmol) and 5(6)-carboxytetramethylrhodamine NHS ester (2 mg, 0.0039 mmol) are dissolved in 140 μL DMF with Et₃N (0.55 μL, 0.0039 mmol) and heated overnight at 31° C. The solvent is evaporated under vacuum and compounds (104) and (105) isolated as a mixture of regioisomers by precipitation in water. ESI-MS: m/z 1046 [M+Na]⁺.

The structural ability of compound (104) and (105) to trigger the formation of a protein dimer is confirmed by in vitro experiments using the fusion protein CLIP-FRB and the fusion protein SNAP-FKBP. 1 μL of a 591 μM solution of FKBP protein fused to a variant of AGT available from Covalys as SNAP26, 10 μL of a 52.9 μM solution of FRB protein fused to a variant of AGT available from Covalys as CLIP and 1 μL of a 62.5 μM solution of mixture of compounds (104) and (105) is added to 3 μL of a solution of 50 mM Tris-HCl pH 7.5; 100 mM NaCl; 0.1% Tween20; 1 mM DTT. Following overnight incubation at 4° C., 15 μL of a solution of 100 mM Tris-HCl pH 6.8; 2% SDS; 35% glycerol; 10 mM EDTA; 20 mM DTT is added. Then the mixture is boiled for 5 min at 95° C. After cooling to rt, 25 μL of this solution is loaded on a 4-20% linear gradient SDS-PAGE gel. After electrophoresis, the proteins are coomassie stained in gel to visualize protein dimer.

### Example 94

### N-Tris{[2-(tert-butoxycarbonyl)ethoxy]methyl}methyl 4-acetylbenzamide (106)

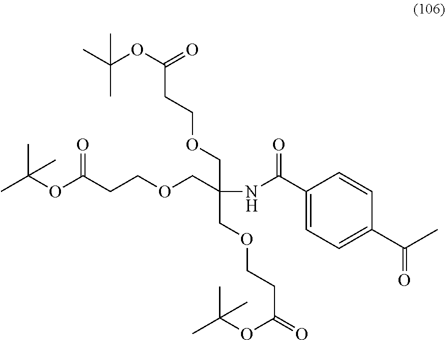

To a solution of 4-acetylbenzoic acid (164 mg, 1 mmol) in DMF (4 mL) PYBOP (520 mg, 1 mmol) is added at rt. The solution is stirred at rt for 20 min. Tris{[2-(tert-butoxy-carbonyl)ethoxy]methyl}methylamine (1) (506 mg, 1 mmol) and DIPEA (165 μL, 1 mmol) are added and the solution is heated at 50° C. for 5 min. The solution is stirred at rt overnight. The solvent is evaporated, the residue is diluted with ethyl acetate (100 mL) and washed with a saturated solution of NaCl. The organic layer is dried over MgSO₄ and concentrated under reduced pressure. Flash chromatography (cyclohexane/ethyl acetate, 2/1→1/1) gives the desired compound (106). ESI-MS: m/z 652.41 [M+H]⁺.

### Example 95

### N-Tris{[2-(tert-butoxycarbonyl)ethoxy]methyl}methyl 4-acetylbenzamide (E)-4-(maleimidomethyl)cyclohexanecarbonylhydrazone (107)

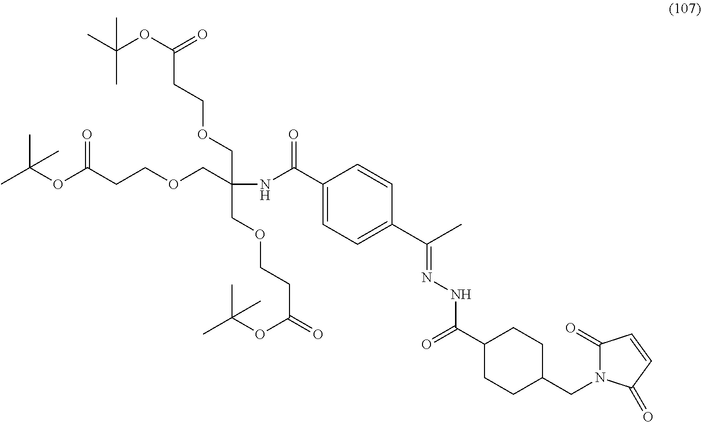

To a solution of compound (106) (180 mg, 0274 mmol) in methanol (10 mL), 4-(male-imidomethyl)cyclohexanecarbonylhydrazide (100 mg, 0.274 mmol) and acetic acid (1 mL) are added at rt. The solution is heated under reflux for 6 h. The solvent is removed under reduced pressure. No further purification is required. MS (ESI) m/z 886 [M+H]⁺.

### Example 96

### N-Tris{[2-carboxyethoxyl]methyl}methyl 4-acetylbenzamide (E)-4-(maleimidomethyl)cyclohexanecarbonylhydrazone (108)

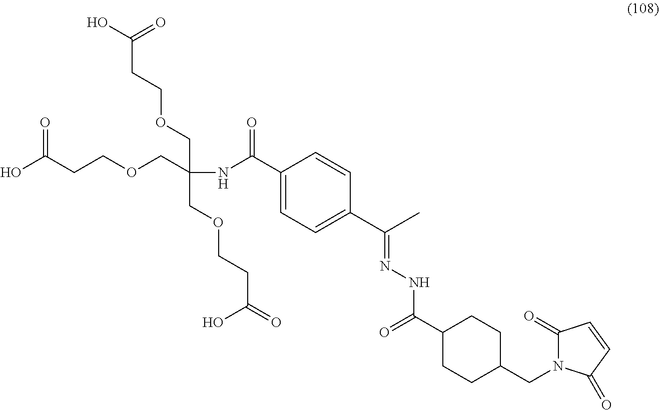

Compound (107) (88 mg, 0.1 mmol) is stirred in 100 μL of 96% formic acid for 18 h. Formic acid is removed at reduced pressure at 5° C. to produce a colorless oil in quantitative yield. MS (ESI) m/z 717 [M+H]⁺.

### Example 97

### N-Tris-{[2-(BG-PEG12-NH-carbonyl)ethoxy]methyl}methyl 4-acetylbenz-amide (E)-4-(maleimidomethyl)cyclohexanecarbonylhydrazone (109)

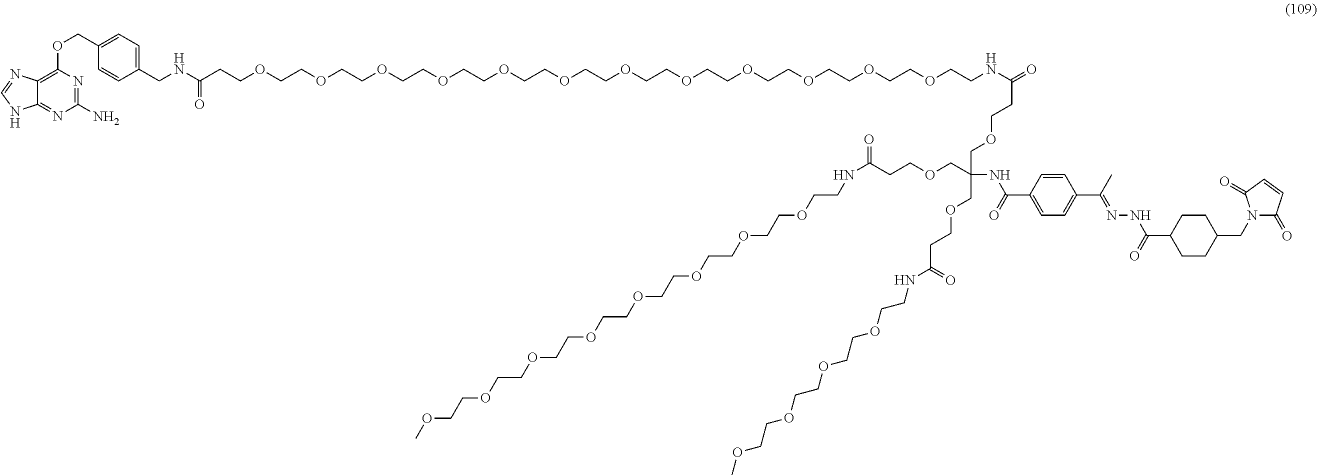

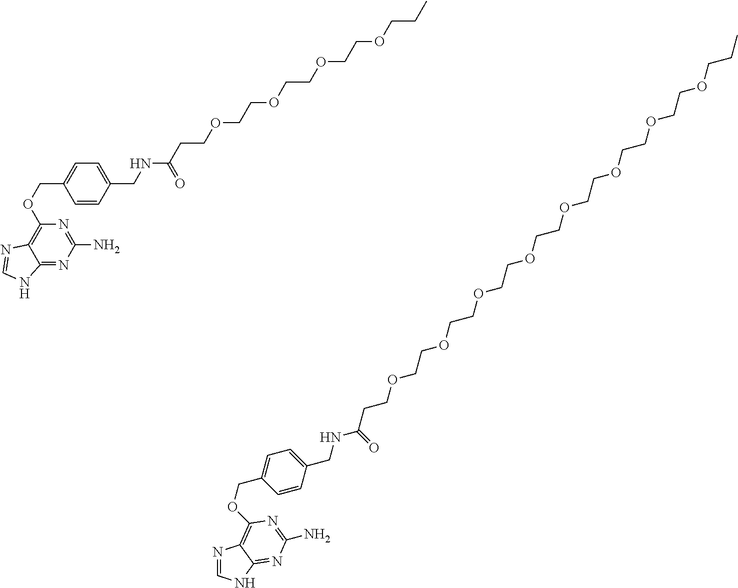

To a solution of compound (108) (11 mg, 0.016 mmol) and BG-PEG12-NH₂ (80) (50 mg, 0.057 mmol, 3.6 eq) in DMF (1 mL) are successively added DIPEA (10 μL, 0.057 mmol, 3.6 eq), HOBT (1 M in NMP, 16 μL, 0.016 mmol, 1 eq) and EDC (2 mg, 0.057 mmol, 3.6 eq) at rt. The resulting mixture is stirred overnight. The solvent is evaporated under vacuum and compound (108) isolated by reversed phase HPLC on a C18 column using a linear gradient of water:acetonitrile (from 95:5 to 20:80 in 20 min, 0.08% TFA). The structural ability of compound (108) to trigger the formation of a protein trimer is confirmed by in vitro experiments using the fusion protein SNAP-FKBP according to Example 85.

1-15. (canceled)

16. A compound of the structure: (substrate)n-linker-(cargo)m or (fusion protein)n-linker-(cargo)m
wherein n=2 or more, the plurality of substrates are specific for same or different enzymes and the plurality of fusion proteins are one or more proteins of interest fused to a plurality of the same or different enzymes which form a covalent bond with the specific substrate;
wherein the linker may be branched or linear and further comprises at least one of
(i) 1 to 300 carbon atoms, wherein up to a third of the carbon atoms may be replaced by at least one of an oxygen, nitrogen or sulfur; and
(ii) double bonds, triple bonds, carbocycles or heterocycles, and may carry further substituents;
wherein m=1 or more and the cargo is selected from a drug and a label detectable by a fluorescence detector, magnetic resonance imaging (MRI), positron emission tomography (PET) or scintigraphy, or a functional group which can be transformed into a drug or a detectable label, and wherein a plurality of cargos comprise the same or different drug, detectable label or functional group;

17. A compound according to claim 16, wherein the substrate is specific for alkylguanine-DNA-alkyltransferase (AGT), alkylcytosine transferase (ACT), acyl carrier protein (ACP), mutant deshalogenase, or mutant serine beta-lactamase.

18. A compound according to claim 16, wherein the linker comprises a straight or branched chain alkylene group with 1 to 300 carbon atoms, wherein optionally
(a) one or more carbon atoms are replaced by oxygen;
(b) one or more carbon atoms are replaced by nitrogen or a nitrogen optionally substituted with a hydrogen atom to form —NH₃ or additionally a carbon substituted by an oxo to form an —NH—CO—;
(c) one or more carbon atoms are replaced by oxygen, and adjacent carbons substituted by oxo to form -0-CO—;
(d) the bond between two adjacent carbon atoms is a double or a triple bond, to form —CH═CH— or —C≡C—;
(e) one or more carbon atoms are replaced by a phenylene, a saturated or unsaturated cycloalkylene, a saturated or unsaturated bicycloalkylene, a bridging heteroaromatic or a bridging saturated or unsaturated heterocyclyl group;
(f) one or more carbon atoms are replaced by a sulfur atom, representing a thioether or, if two adjacent carbon atoms are replaced by sulfur atoms, a disulfide linkage —S—S—; or
(g) a combination of two or more alkylene and/or modified alkylene groups according to (a) to (f), optionally substituted.

19. A compound according to claim 16, 17 or 18 wherein n is 2 or 3 and m is 1 or 2.

20. The compound according to claim 16, wherein the protein of interest is a recombinant antibody fragment.

21. A pharmaceutical composition comprising a compound according to claim 16, wherein at least one of the cargo entities is a drug.

22. A method of treatment of cancer comprising administering a compound according to claim 16, wherein at least one of the cargo entities is a drug, to a patient in need thereof.
